(19) United States
(12) Patent Application Publication
(10) Pub. No.: US 2005/0256037 A1
(43) Pub. Date: Nov. 17, 2005

(54) Antibacterial amide macrocyles

(75) Inventors: Thomas Lampe, Dusseldorf (DE); Isabelle Adelt, Dusseldorf (DE); Dieter Beyer, Wuppertal (DE); Nina Brunner, Essen (DE); Rainer Endermann, Wuppertal (DE); Kerstin Ehlert, Velbert (DE); Hein-Peter Kroll, Wuppertal (DE); Franz Von Nussbaum, Dusseldorf (DE); Siegfried Raddatz, Koln (DE); Joachim Rudolph, Guilford, CT (US); Guido Schiffer, Wuppertal (DE); Andreas Schumacher, Efringen-Kirchen (DE); Yolanda Cancho-Grande, Hilden (DE); Martin Michels, Solingen (DE); Stefan Weigand, Wuppertal (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/957,489

(22) Filed: Oct. 1, 2004

(30) Foreign Application Priority Data
Oct. 1, 2003 (DE) 10345724.0
Dec. 16, 2003 (DE) 10358822.1

(51) Int. Cl. A61K 38/12; C07K 5/12

(52) U.S. Cl. 514/9; 530/317; 540/460

(57) ABSTRACT

The invention relates to antibacterial amide macrocycles and process for their preparation, their use for the treatment and/or prophylaxis of diseases, and to their use for producing medicaments for the treatment and/or prophylaxis of diseases, especially of bacterial infections.

The invention relates to anti bacterial amide macrocycles and process for their preparation, their use for the treatment and/or prophylaxis of diseases, and to their use for producing medicaments for the treatment and/or prophylaxis of diseases, especially of bacterial infections.

U.S. Pat. No. 3,452,136, thesis of R. U. Meyer, Stuttgart University, Germany 1991, thesis of V. Leitenberger, Stuttgart University, Germany 1991, Synthesis (1992), (10), 1025-30, J. Chem. Soc., Perkin Trans. 1 (1992), (1), 123-130, J. Chem. Soc., Chem. Commun. (1991), (10), 744, Synthesis (1991), (5), 409-13, J. Chem. Soc., Chem. Commun. (1991), (5), 275-7, J. Antibiot. (1985), 38(11), 1462-8, J. Antibiot. (1985), 38(11), 1453-61, describe the natural product biphenomycin B as having antibacterial activity. Some steps in the synthesis of biphenomycin B are described in Synlett (2003), 4, 522-526.

Chirality (1995), 7(4), 181-92, J. Antibiot. (1991), 44(6), 674-7, J. Am. Chem. Soc. (1989), 111(19), 7323-7, J. Am. Chem. Soc. (1989), 111(19), 7328-33, J. Org. Chem. (1987), 52(54), 5435-7, Anal. Biochem. (1987), 165(1), 108-13, J. Org. Chem. (1985), 50(8), 1341-2, J. Antibiot. (1993), 46(3), C-2, J. Antibiot. (1993), 46(1), 135-40, Synthesis (1992), (12), 1248-54, Appl. Environ. Microbiol. (1992), 58(12), 3879-8, J. Chem. Soc., Chem. Commun. (1992), (13), 951-3 describe a structurally related natural product, biphenomycin A, which has a further substitution with a hydroxy group on the macrocycle.

The natural products do not comply in terms of their properties with the requirements for antibacterial medicaments. Although structurally different agents with antibacterial activity are available on the market, development of resistance is a regular possibility. Novel agents for good and more effective therapy are therefore desirable.

One object of the present invention is therefore to provide novel and alternative compounds having the same or improved antibacterial effect for the treatment of bacterial diseases in humans and animals.

It has surprisingly been found that certain derivatives of these natural products in which the carboxyl group of the natural product is replaced by an amide group comprising a basic group have antibacterial activity on S. aureus strains (RN4220Bi^{R} and T17) which are resistant to biphenomycin.

In addition, the derivatives show an improved spontaneous resistance rate against S. aureus wild-type strains and biphenomycin-resistant S. aureus strains.

The invention relates to compounds of the formula 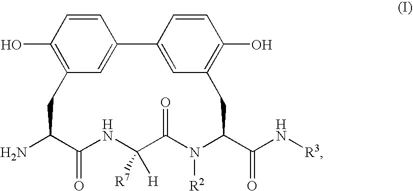
in which
R⁷ is a group of the formula 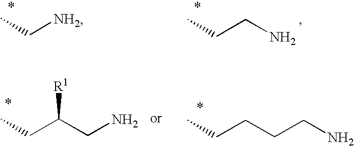
   where
   R¹ is hydrogen or hydroxy,
   * is the point of attachment to the carbon atom,
R² is hydrogen, methyl or ethyl,
R³ is a group of the formula 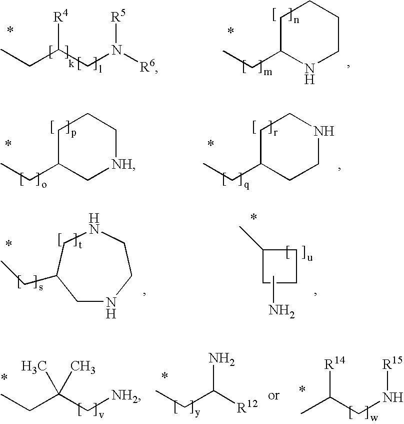
   where
   * is the point of attachment to the nitrogen atom,
   R⁴ is hydrogen or hydroxy,
   R⁵ and R¹⁵ are independently of one another hydrogen, methyl or a group of the formula 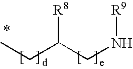
      in which
      * is the point of attachment to the nitrogen atom,
      R⁸ is hydrogen or *-(CH₂)_{f}—NHR¹⁰,
         in which
         R¹⁰ is hydrogen or methyl,
         and
         f is a number 1,2 or 3,
      R⁹ is hydrogen or methyl,
      d is a number 0, 1, 2 or 3,
      and
      e is a number 1, 2 or 3,
   R⁶ is hydrogen or aminoethyl,
   or
   R⁵ and R⁶ form together with the nitrogen atom to which they are bonded a piperazine ring,
   R¹²and R¹⁴ are independently of one another a group of the formula
      *-(CH₂)_{Z1}—OH or *-(CH₂)_{Z2}—NHR¹³,
      in which
      * is the point of attachment to the carbon atom,
      Z1 and Z2 are independently of one another a number 1, 2, 3 or 4,
      R¹³ is hydrogen or methyl,
   k and t are independently of one another a number 0 or 1,
   l, w and y are independently of one another a number 1, 2, 3 or 4,
   m, r, w and v are independently of one another a number 1 or 2,
   n, o, p and q are independently of one another a number 0, 1 or 2,
   u is a number 0, 1, 2 or 3, 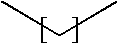
   w or y may independently of one another when w or y is 3 carry a hydroxy group on the middle carbon atom of the three-membered chain,
      and the salts thereof, the solvates thereof and the solvates of the salts thereof.

Compounds of the invention are the compounds of the formula (I) and the salts, solvates and solvates of the salts thereof, and the compounds which are encompassed by formula (I) and are mentioned below as exemplary embodiment(s), and the salts, solvates and solvates of the salts thereof, where the compounds which are encompassed by formula (I) and are mentioned below are not already salts, solvates and solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated from such mixtures of enantiomers and/or diastereomers by known processes such as chromatography on a chiral phase or crystallization using chiral amines or chiral acids.

The invention also relates, depending on the structure of the compounds, to tautomers of the compounds.

Salts preferred for the purposes of the invention are physiologically acceptable salts of the compounds of the invention.

Physiologically acceptable salts of the compounds (I) include acid addition salts of mineral acids, carboxylic acids and sulphonic acids, e.g. salts of hydrochloric acid, hydrobromic acid, sulphuric acid, phosphoric acid, methanesulphonic acid, ethanesulphonic acid, toluenesulphonic acid, benzenesulphonic acid, naphthalene-disulphonic acid, acetic acid, propionic acid, lactic acid, tartaric acid, malic acid, citric acid, fumaric acid, maleic acid, trifluoroacetic acid and benzoic acid.

Physiologically acceptable salts of the compounds (I) also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 carbon atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dehydroabietylamine, arginine, lysine, ethylenediamine and methylpiperidine.

Solvates refer for the purposes of the invention to those forms of the compounds which form a complex in the solid or liquid state through coordination with solvent molecules. Hydrates are a special form of solvates in which coordination takes place with water.

A # symbol on a carbon atom means that the compound is, in terms of the configuration at this carbon atom, in enantiopure form, by which is meant for the purposes of the present invention an enantiomeric excess of more than 90% (>90% ee).

In the formulae of groups for which R³ can stand, the end of the line besides which there is an * in each case does not represent a carbon atom or a CH₂ group but is part of the bond to the nitrogen atom to which R³ is bonded. R³ is thus for example 2-aminoethyl in the case of k=0, l=1 and R⁵═H, 3-amino-2-hydroxypropyl in the case of k=1, R⁴═OH, l=1 and R⁵═H, piperidin-4-ylmethyl in the case of q=1 and r=1 or piperidin-4-yl in the case of q=0 and r=1.

In the formulae of the groups for which R⁷ can stand, the end of the line besides which there is an * in each case does not represent a carbon atom or a CH₂ group but is part of the bond to the carbon atom to which R⁷ is bonded.

Preference is given for the purposes of the present invention to compounds of the formula (I) in which
R⁷ is a group of the formula 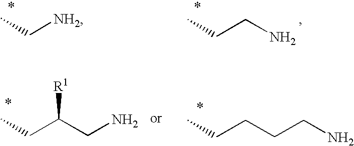
   where
   R¹ is hydrogen or hydroxy,
   * is the point of attachment to the carbon atom,
R² is hydrogen, methyl or ethyl,
R³ is a group of the formula 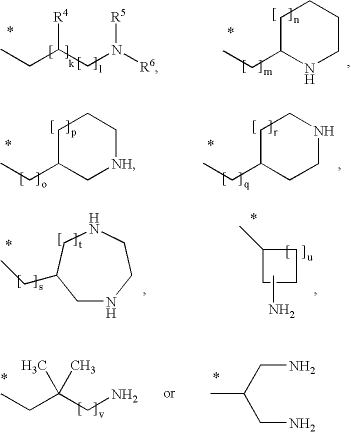
   where
   R⁴ is hydrogen or hydroxy,
   R⁵ is hydrogen or methyl,
   R⁶ is hydrogen,
   or
   R⁵ and R⁶ form together with the nitrogen atom to which they are bonded a piperazine ring
   k and t are independently of one another a number 0 or 1,
   l is a number 1, 2, 3 or 4,
   m, r, s and v are independently of one another a number 1 or 2,
   n, o, p and q are independently of one another a number 0, 1 or 2,
   u is a number 0, 1, 2 or 3,
   * is the point of attachment to the nitrogen atom,
      and the salts thereof, the solvates thereof and the solvates of the salts thereof.

Preference is given for the purposes of the present invention also to compounds of the formula 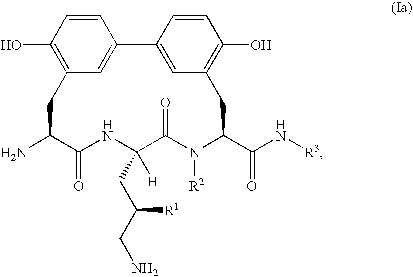
in which
R¹ is hydrogen or hydroxy,
R² is hydrogen or methyl,
R³ is a group of the formula 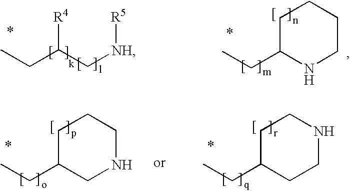
   where
   R⁴ is hydrogen or hydroxy,
   R⁵ is hydrogen or methyl,
   k is a number 0 or 1,
   l, m and r are independently of one another a number 1 or 2,
   n, o, p and q are independently of one another a number 0, 1 or 2,
   * is the point of attachment to the nitrogen atom,
      and the salts thereof, solvates thereof and the solvates of the salts thereof.

Preference is given for the purposes of the present invention also to compounds of the formula (Ia) in which
R¹ is hydrogen or hydroxy,
R² is hydrogen or methyl,
R³ is a group of the formula 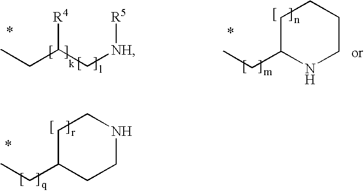
   where
   R⁴ is hydrogen or hydroxy,
   R⁵ is hydrogen or methyl,
   k is a number 0 or 1,
   l, m and r are independently of one another a number 1 or 2,
   n and q are independently of one another a number 0, 1 or 2,
   * is the point of attachment to the nitrogen atom,
      and the salts thereof, the solvates thereof and the solvates of the salts thereof.

Preference is given for the purposes of the present invention also to compounds of the formula (Ia) in which
R¹ is hydrogen or hydroxy,
R² is hydrogen or methyl,
R³ is a group of the formula 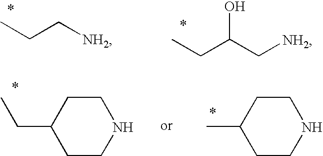
   where
   * is the point of attachment to the nitrogen atom,
      and the salts thereof, the solvates thereof and the solvates of the salts thereof.

Preference is given for the purposes of the present invention also to compounds of the formula (I) in which
R⁷ is a group of the formula 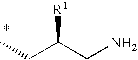
   where
   R¹ is hydrogen or hydroxy,
   * is the point of attachment to the carbon atom,
R² is hydrogen, methyl or ethyl,
R³ is a group of the formula 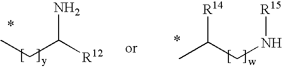
where
* is the point of attachment to the nitrogen atom,
R¹⁵ is hydrogen, methyl or a group of the formula 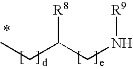
   in which
   * is the point of attachment to the nitrogen atom,
   R⁸ is hydrogen or *-(CH₂)_{f}—NHR¹⁰,
      where
      R¹⁰ is hydrogen or methyl,
      and
      f is a number 1, 2 or 3,
   R⁹ is hydrogen or methyl,
   d is a number 0, 1, 2 or 3,
   and
   e is a number 1, 2 or 3,
R¹² and R¹⁴ are independently of one another a group of the formula *-(CH₂)_{Z1}—OH or *-(CH₂)_{Z2}—NHR¹³
   in which
   * is the point of attachment to the carbon atom,
   Z1 and Z2 are independently of one another a number 1, 2, 3 or 4,
   R¹³ is hydrogen or methyl,
w and y are independently of one another a number 1, 2, 3 or 4,
   and the salts thereof, the solvates thereof and the solvates of the salts thereof.

Preference is given for the purposes of the present invention also to compounds of the formula (I) or (Ia) in which R³ is 2-aminoeth-1-yl, 3-aminoprop-1-yl, 4-aminobut-1-yl, 5-aminopent-1-yl, 2-(methylamino)eth-1-yl, 3-amino-2-hydroxyprop-1-yl, 3-amino-2,2-dimethylprop-1-yl, 2-amino-1-(aminomethyl)eth-1-yl, 3-amino-1-(hydroxymethyl)prop-1-yl, 4-amino-1-(hydroxymethyl)but-1-yl, 4-amino-1-(hydroxyethyl)but-1-yl, 2,3-diaminoprop-1-yl, 2,4-diaminobut-1-yl, 2,5-diaminopent-1-yl, 2,6-diaminohex-1-yl, 3-amino-4-hydroxybut-1-yl, 4-amino-5-hydroxypent-1-yl, 4-amino-6-hydroxyhex-1-yl, 5-amino-6-hydroxyhex-1-yl, 2-(aminoethylamino)eth-1-yl, 3-(3-aminoprop-1-ylamino)prop-1-yl, 3-(1,3-diaminoprop-2-ylamino)prop-1-yl, (diaminoethylamino)eth-1-yl, 2-(piperazin-1-yl)eth-1-yl, 3-(piperazin-1-yl)-2-hydroxyprop-1-yl, (pyrrolidin-2-yl)methyl, piperidin-4-yl, (piperidin-2-yl)methyl, (piperidin-3-yl)methyl, (piperidin-4-yl)methyl, 2-(piperidin-2-yl)ethyl, (azepan-2-yl)methyl, 2-aminocycloprop-1-yl, 2-aminocyclohex-1-yl, 3-aminocyclohex-1-yl or (1,4-diazepan-6-yl)methyl.

Particular preference is given for the purposes of the present invention also to compounds of the formula (I) or (Ia) in which R³ is 2-aminoeth-1-yl, 3-(3-aminoprop-1-ylamino)prop-1-yl, (diaminoethylamino)eth-1-yl or 2,5-diaminopent-1-yl.

Very particular preference is given for the purposes of the present invention also to compounds of the formula (I) or (Ia) in which R³ is 2-aminoeth-1-yl.

Particular preference is given to the compound (8S,11S,14S)-14-amino-N-(2-aminoethyl)-11-[(2R)-3-amino-2-hydroxypropyl]-5,17-dihydroxy-9-methyl-10,13-dioxo-9,12-diazatricyclo[14.3.1.1.^{2,6}]henicosa-1(20),2(21),3,5,16,18-hexaene-8-carboxamide of the formula 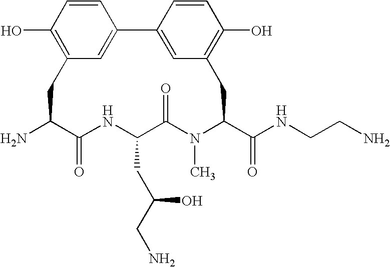
and its trihydrochloride and its other salts, its solvates and the solvates of its salts. The trihydrochloride is described in Example 1.

Particular preference is also given to the compound (8S,11S,14S)-14-amino-N-(2-aminoethyl)-11-(3-aminopropyl)-5,17-dihydroxy-10,1 3-dioxo-9,12-diazatricyclo[14.3.1.1^{2,6}]henicosa-1(20),2(21),3,5,16,18-hexaene-8-carboxamide of the formula 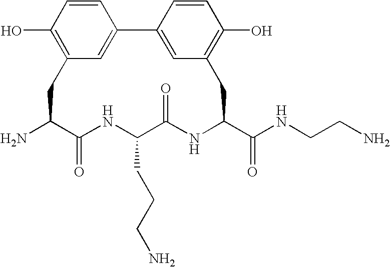
and its trihydrochloride and its other salts, its solvates and the solvates of its salts. The compound is described in Example 14 and its trihydrochloride in Example 6.

Particular preference is also given to the compound (8S,11S,14S)-14-amino-11-(3-aminopropyl)-N-{3-[(3-aminopropyl)amino]propyl}-5,17-dihydroxy-10,13-dioxo-9,12-diazatricyclo[14.3.1.1^{2,6}]henicosa-1(20),2(21),3,5,16,18-hexaene-8-carboxamide of the formula 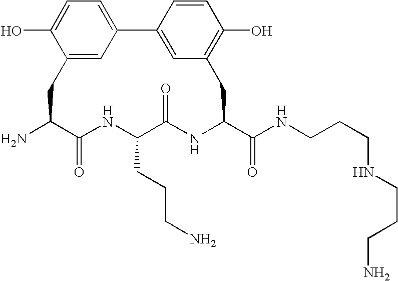
and its tetrahydrochloride and its other salts, its solvates and the solvates of its salts. The tetrahydrochloride is described in Example 42.

Particular preference is also given to the compound (8S,11S,14S)-14-amino-11-(3-aminopropyl)-N-{2-[bis(2-aminoethyl)amino]ethyl}-5,17-dihydroxy-10,13-dioxo-9,12-diazatricyclo[14.3.1.1^{2,6}]henicosa-1(20),2(21),3,5,16,18-hexaene-8-carboxamide of the formula 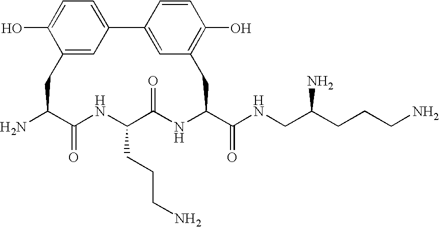
and its tetrahydrochloride and its other salts, its solvates and the solvates of its salts. The tetrahydrochloride is described in Example 43.

Particular preference is also given to the compound (8S,11S,14S)-14-amino-11-(3-aminopropyl)-N-[(2S)-2,5-diaminopentyl]-5,17-dihydroxy-10,13-dioxo-9,12-diazatricyclo[14.3.1.1 ^{2,6}]henicosa-1(20),2(21),3,5,16,18,-hexaene-8-carboxamide of the formula 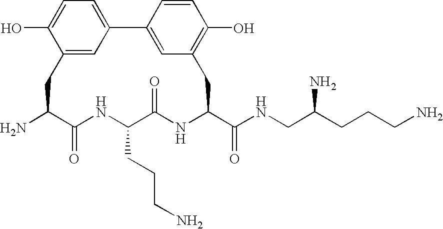
and its tetrahydrochloride and its other salts, its solvates and the solvates of its salts. The tetrahydrochloride is described in Example 45.

The invention further relates to a process for preparing the compounds of the formula (I) or their salts, their solvates or the solvates of their salts, where in process
[A] compounds of the formula 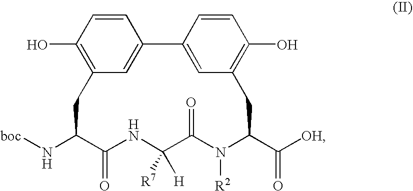
in which R² and R⁷ have the meaning indicated above, and boc is tert-butoxycarbonyl, are reacted in a two-stage process firstly in the presence of one or more dehydrating reagents with compounds of the formula
   H₂NR³ (III)
in which R³ has the meaning indicated above,
and subsequently with an acid,
or
[B] compounds of the formula 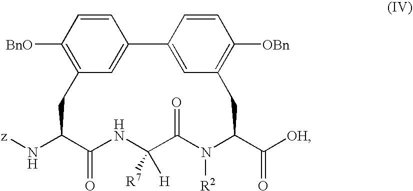
in which R² and R⁷ have the meaning indicated above, and Z is benzyloxycarbonyl,
are reacted in a two-stage process firstly in the presence of one or more dehydrating reagents with compounds of the formula
   H₂NR³ (III)
in which R³ has the meaning indicated above,
and subsequently with an acid or by hydrogenolysis.

The free base of the salts can be obtained for example by chromatography on a reversed phase column with an acetonitrile/water gradient with addition of a base, in particular by use of an RP18 Phenomenex Luna C18(2) column and diethylamine as base.

The invention further relates to a process for preparing the compounds of the formula (I) or their solvates according to claim 1, in which salts of the compounds or solvates of the salts of the compounds are converted into the compounds by chromatography with addition of a base.

The hydroxy group on R¹ is, where appropriate, protected during the reaction with compounds -of the formula (III) with a tert-butyldimethylsilyl group, which is eliminated in the second reaction step.

Reactive functionalities in the radicals R³ and R⁷ of compounds of the formulae (II), (III), (VI), (VIII), (IX) and (XI) are introduced already protected into the synthesis, with preference for acid-labile protective groups (e.g. boc or Z). After reaction has taken place to give compounds of the formula (I), the protective groups can be eliminated by deprotection reaction. This takes place by standard methods of protective group chemistry. Deprotection reactions under acidic conditions or by hydrogenolysis are preferred.

The reaction in the first stage of processes [A] and [B] generally takes place in inert solvents, where appropriate in the presence of a base, preferably in a temperature range from 0° C. to 40° C. under atmospheric pressure.

Dehydrating reagents suitable in this connection are, for example, carbodiimides such as, for example, N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium-3′-sulphonate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethyl-amino)phosphonium hexafluorophosphate (BOP), or mixtures of the latter, or mixture of the latter together with bases.

Bases are, for example, alkali metal carbonates such as, for example, sodium or potassium carbonate, or bicarbonate, or organic bases such as trialkylamines, e.g. triethylamine, N-methylmorpholine, N-methylpiperidine, 4-dimethylaminopyridine or diisopropylethylamine.

The condensation is preferably carried out with HATU in the presence of a base, in particular diisopropylethylamine, or with HOBt and EDC.

Inert solvents are, for example, halohydrocarbons such as dichloromethane or trichloromethane, hydrocarbons such as benzene, or nitromethane, dioxane, dimethylformamide or acetonitrile. It is likewise possible to employ mixtures of these solvents. Dimethylformamide is particularly preferred.

The reaction with an acid in the second stage of processes [A] and [B] preferably takes place in a temperature range from 0° C. to 40° C. under atmospheric pressure.

Acids suitable in this connection are hydrogen chloride in dioxane, hydrogen bromide in acetic acid or trifluoroacetic acid in methylene chloride.

The hydrogenolysis in the second stage of process [B] generally takes place in a solvent in the presence of hydrogen and palladium on activated carbon, preferably in a temperature range from 0° C. to 40° C. under atmospheric pressure.

Solvents are, for example, alcohols such as methanol, ethanol, n-propanol or isopropanol, in a mixture with water and glacial acetic acid, with preference for a mixture of ethanol, water and glacial acetic acid.

The compounds of the formula (III) are known or can be prepared in analogy to known processes.

The compounds of the formula (II) are known or can be prepared by reacting compounds of the formula 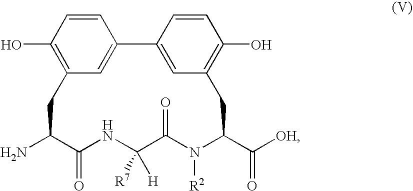
in which R² and R⁷ have the meaning indicated above,
with di(tert-butyl)dicarbonate in the presence of a base.

The reaction generally takes place in a solvent, preferably in a temperature range from 0° C. to 40° C. under atmospheric pressure.

Bases are, for example, alkali metal hydroxides such as sodium or potassium hydroxide, or alkali metal carbonates such as caesium carbonate, sodium or potassium carbonate, or other bases such as DBU, triethylamine or diisopropylethylamine, with preference for sodium hydroxide or sodium carbonate.

Solvents are, for example, halohydrocarbons such as methylene chloride or 1,2-dichloroethane, alcohols such as methanol, ethanol or isopropanol, or water.

The reaction is preferably carried out with sodium hydroxide in water or sodium carbonate in methanol.

The compounds of the formula (V) are known or can be prepared by reacting compounds of the formula 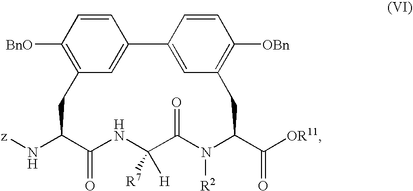
in which R² and R⁷ have the meaning indicated above, and
R¹¹ is benzyl, methyl or ethyl,
with an acid or by hydrogenolysis as described for the second stage of process [B], where appropriate by subsequent reaction with a base to hydrolyse the methyl or ethyl ester.

The hydrolysis can take place for example as described for the reaction of compounds of the formula (VI) to give compounds of the formula (IV).

The compounds of the formula (IV) are known or can be prepared by hydrolysing the benzyl, methyl or ethyl ester in compounds of the formula (VI).

The reaction generally takes place in a solvent in the presence of a base, preferably in a temperature range from 0° C. to 40° C. under atmospheric pressure.

Bases are, for example, alkali metal hydroxides such as lithium, sodium or potassium hydroxide, with preference for lithium hydroxide.

Solvents are, for example, halohydrocarbons such as dichloromethane or trichloromethane, ethers such as tetrahydrofuran or dioxane, or alcohols such as methanol, ethanol or isopropanol, or dimethylformamide. It is likewise possible to employ mixtures of the solvents or mixtures of the solvents with water. Tetrahydrofuran or a mixture of methanol and water are particularly preferred.

The compounds of the formula (VI) are known or can be prepared by reacting compounds of the formula 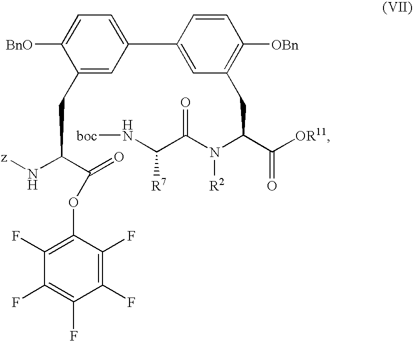
in which R², R⁷ and R¹¹ have the meaning indicated above,
in the first stage with acids as described for the second stage of processes [A] and [B], and in the second stage with bases.

The reaction with bases in the second stage generally takes place in a solvent, preferably in a temperature range from 0° C. to 40° C. under atmospheric pressure.

Bases are, for example, alkali metal hydroxides such as sodium or potassium hydroxide, or alkali metal carbonates such as caesium carbonate, sodium or potassium carbonate, or other bases such as DBU, triethylamine or diisopropyl-ethylamine, with preference for triethylamine.

Solvents are, for example, halohydrocarbons such as chloroform, methylene chloride or 1,2-dichloroethane, or tetrahydrofuran, or mixtures of these solvents, with preference for methylene chloride or tetrahydrofuran.

The compounds of the formula (VII) are known or can be prepared by reacting compounds of the formula 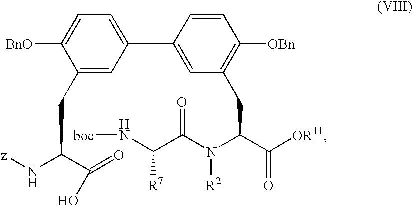
in which R², R⁷ and R¹¹ have the meaning indicated above,
with pentafluorophenol in the presence of dehydrating reagents as described for the first stage of processes [A] and [B].

The reaction preferably takes place with DMAP and EDC in dichloromethane in a temperature range from −40° C. to 40° C. under atmospheric pressure.

The compounds of the formula (VIII) are known or can be prepared by reacting compounds of the formula 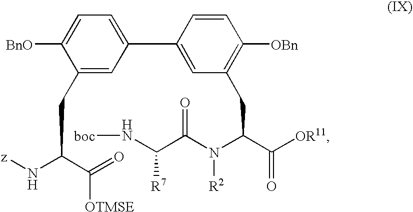
in which R², R⁷ and R¹¹ have the meaning indicated above,
with fluoride, in particular with tetrabutylammonium fluoride.

The reaction generally takes place in a solvent, preferably in a temperature range from −10° C. to 30° C. under atmospheric pressure.

Examples of inert solvents are halohydrocarbons such as dichloromethane, or hydrocarbons such as benzene or toluene, or ethers such as tetrahydrofuran or dioxane, or dimethylformamide. It is likewise possible to employ mixtures of the solvents. Preferred solvents are tetrahydrofuran and dimethylformamide.

The, compounds of the formula (IX) are known or can be prepared by reacting compounds of the formula 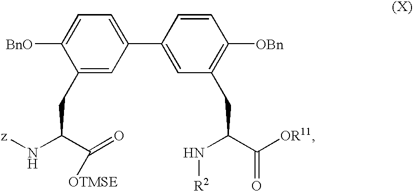
in which R² and R¹¹ have the meaning indicated above,
with compounds of the formula 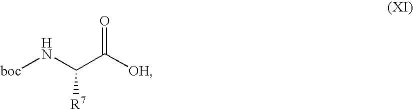
in which R⁷ has the meaning indicated above,
in the presence of dehydrating reagents as described for the first stage of processes [A] and [B].

The compounds of the formula (X) are known or can be prepared in analogy to the processes described in the examples section.

The compounds of the formula (XI) are known or can be prepared in analogy to known processes.

The compounds of the invention show a valuable range of pharmacological and pharmacokinetic effects which could not have been predicted.

They are therefore suitable for use as medicaments for the treatment and/or prophylaxis of diseases in humans and animals.

The compounds of the invention can, because of their pharmacological properties, be employed alone or in combination with other active ingredients for the treatment and/or prophylaxis of infectious diseases, especially of bacterial infections.

For example, it is possible to treat and/or prevent local and/or systemic diseases caused by the following pathogens or by mixtures of the following pathogens:
gram-positive cocci, e.g. staphylococci (Staph. aureus, Staph. epidermidis) and streptococci (Strept. agalactiae, Strept. faecalis, Strept. pneumoniae, Strept. pyogenes); gram-negative cocci (Neisseria gonorrhoeae) and gram-negative rods such as enterobacteriaceae, e.g. Escherichia coli, Haemophilus influenzae, Citrobacter (Citrob. freundii, Citrob. diversus), Salmonella and Shigella; also klebsiellas (Klebs. pneumoniae, Klebs. oxytoca), Enterobacter (Ent. aerogenes, Ent. agglomerans), Haffiia, Serratia (Serr. marcescens), Proteus (Pr. mirabilis, Pr. rettgeri, Pr. vulgaris), Providencia, Yersinia, and the genus Acinetobacter. The antibacterial range additionally includes the genus Pseudomonas (Ps. aeruginosa, Ps. maltophilia) and strictly anaerobic bacteria such as Bacteroides fragilis, representatives of the genus Peptococcus, Peptostreptococcus, and the genus Clostridium; also mycoplasmas (M. pneumoniae, M. hominis, M. urealyticum) and mycobacteria, e.g. Mycobacterium tuberculosis.

The above list of pathogens is merely by way of example and is by no means to be interpreted restrictively. Examples which may be mentioned of diseases which are caused by the pathogens mentioned or mixed infections and can be prevented, improved or healed by preparations of the invention, which can be used topically, are:
infectious diseases in humans such as, for example, septic infections, bone and joint infections, skin infections, postoperative wound infections, abscesses, phlegmon, wound infections, infected burns, burn wounds, infections in the oral region, infections after dental operations, septic arthritis, mastitis, tonsillitis, genital infections and eye infections.

Apart from humans, bacterial infections can also be treated in other species. Examples which may be mentioned are:
Pigs: coli diarrhoea, enterotoxaemia, sepsis, dysentery, salmonellosis, mastitis-metritis-agalactia syndrome, mastitis;
Ruminants (cattle, sheep, goats): diarrhoea, sepsis, bronchopneumonia, salmonellosis, pasteurellosis, mycoplasmosis, genital infections;
Horses: bronchopneumonias, joint ill, puerperal and postpuerperal infections, salmonellosis;
Dogs and cats: bronchopneumonia, diarrhoea, dermatitis, otitis, urinary tract infections, prostatitis;
Poultry (chickens, turkeys, quail, pigeons, ornamental birds and others): mycoplasmosis, E. coli infections, chronic airway diseases, salmonellosis, pasteurellosis, psittacosis.

It is likewise possible to treat bacterial diseases in the rearing and management of productive and ornamental fish, in which case the antibacterial spectrum is extended beyond the pathogens mentioned above to further pathogens such as, for example, Pasteurella, Brucella, Campylobacter, Listeria, Erysipelothrix, corynebacteria, Borrelia, Treponema, Nocardia, Rikettsia, Yersinia.

The present invention further relates to the use of the compounds of the invention for the treatment and/or prophylaxis of diseases, preferably of bacterial diseases, especially of bacterial infections.

The present invention further relates to the use of the compounds of the invention for the treatment and/or prophylaxis of diseases, especially of the aforementioned diseases.

The present invention further relates to the use of the compounds of the invention for producing a medicament for the treatment and/or prophylaxis of diseases, especially of the aforementioned diseases.

The present invention further relates to a method for the treatment and/or prophylaxis of diseases, especially of the aforementioned diseases, by use of an antibacterially effective amount of the compounds of the invention.

The compounds of the invention may act systemically and/or locally. For this purpose, they can be administered in a suitable way such as, for example, by the oral, parenteral, pulmonary, nasal, sublingual, lingual, buccal, rectal, dermal, transdermal, conjuctival or otic route or as implant or stent.

The compounds of the invention can be administered in administration forms suitable for these administration routes.

Suitable for oral administration are administration forms which function according to the prior art and deliver the compounds of the invention rapidly and/or in modified fashion, and which contain the compounds of the invention in crystalline and/or amorphized and/or dissolved form, such as, for example, tablets (uncoated or coated tablets, for example having coatings which are resistant to gastric juice or are insoluble or dissolve with a delay and control the release of the compound of the invention), tablets which disintegrate rapidly in the mouth, or films/wafers, films/lyophilisates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, preparations for injection and infusion in the form of solutions, suspensions, emulsions, lyophilisates or sterile powders.

Suitable for the other administration routes are, for example, pharmaceutical forms for inhalation (inter alia powder inhalers, nebulizers), nasal drops, solutions, sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention can be converted into the stated administration forms. This can take place in a manner known per se by mixing with inert, nontoxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (e.g. antioxidants such as, for example, ascorbic acid), colours (e.g. inorganic pigments such as, for example, iron oxides) and masking tastes and/or odours.

The present invention further relates to medicaments which comprise at least one compound of the invention, normally together with one or more inert, nontoxic, pharmaceutically suitable excipients, and to the use thereof for the aforementioned purposes.

It has generally proved advantageous on parenteral administration to administer amounts of about 5 to 250 mg/kg of body weight per 24 h to achieve effective results. The amount on oral administration is about 5 to 100 mg/kg of body weight per 24 h.

It may nevertheless be necessary where appropriate to deviate from the stated amounts, in particular as a function of the body weight, administration route, individual behaviour towards the active ingredient, nature of the preparation and time or interval over which administration takes place. Thus, it may be sufficient in some cases to make do with less than the aforementioned minimum amount, whereas in other cases the stated upper limit must be exceeded. Where larger amounts are administered, it may be advisable to divide these into a plurality of single doses over the day.

The percentage data in the following tests and examples are percentages by weight unless otherwise indicated; parts are parts by weight. Solvent ratios, dilution ratios and concentration data for liquid/liquid solutions are in each case based on volume.

### A. EXAMPLES

Abbreviations Used:

| | |
|---|---|
| abs. | absolute |
| aq. | aqueous |
| Bn | benzyl |
| boc | tert-butoxycarbonyl |
| CDCl₃ | deuterochloroform |
| CH | cyclohexane |
| conc. | concentrated |
| d | doublet (in ¹H NMR) |
| dd | doublet of doublets (in ¹H NMR) |
| DCC | dicyclohexylcarbodiimide |
| DIC | diisopropylcarbodiimide |
| DIEA | diisopropylethylamine (Hunig's base) |
| DMSO | dimethyl sulphoxide |
| DMAP | 4-N,N-dimethylaminopyridine |
| DMF | dimethylformamide |
| EA | ethyl acetate (acetic acid ethyl ester) |
| EDC | N′-(3-dimethylaminopropyl)-N-ethylcarbodiimide × HCl |
| ESI | electrospray ionization (in MS) |
| HATU | O-(7-azabenzotriazol-1-yl)-N,N,N′,N′-tetramethyluronium |
| | hexafluorophosphate |
| HBTU | O-(benzotriazol-1-yl)-N,N,N′N′-tetramethyluronium |
| | hexafluorophosphate |
| HOBt | 1-hydroxy-1H-benzotriazole × H₂O |
| h | hour(s) |
| HPLC | high pressure, high performance liquid chromatography |
| LC-MS | coupled liquid chromatography-mass spectroscopy |
| m | multiplet (in ¹H NMR) |
| min | minute |
| MS | mass spectroscopy |
| NMR | nuclear magnetic resonance spectroscopy |
| MTBE | methyl tert-butyl ether |
| Pd/C | palladium/carbon |
| q | quartet (in ¹H NMR) |
| R_{f} | retention index (in TLC) |
| RP | reverse phase (in HPLC) |
| RT | room temperature |
| Rₜ | retention time (in HPLC) |
| s | singlet (in ¹H NMR) |
| sat | saturated |
| t | triplet (in ¹H NMR) |
| TBS | tert-butyldimethylsilyl |
| TFA | trifluoroacetic acid |
| THF | tetrahydrofuran |
| TLC | thin layer chromatography |
| TMSE | 2-(trimethylsilyl)ethyl |
| TPTU | 2-(2-oxo-1(2H)-pyridyl)-1,1,3,3,-tetramethyluronium |
| | tetrafluoroborate |
| Z | benzyloxycarbonyl |

LC-MS and HPLC Methods:

Method 1 (HPLC): Instrument: HP 1100 with DA detection; column: Kromasil RP-18, 60 mm×2 mm, 3.5 μm; eluent A: 5 ml of perchloric acid/l of water, eluent B: acetonitrile; gradient: 0 min 2% B, 0.5 min 2% B, 4.5 min 90% B, 6.5 min 90% B; flow rate: 0.75 ml/min; oven: 30° C. UV detecifon: 210 nm.

Method 2 (LC-MS): Instrument: Micromass Platform LCZ; column: Symmetry C18, 50 mm×2.1 mm, 3.5 μm; temperature: 40° C.; flow rate: 0.5 ml/min; eluent A: acetonitrile+0.1% formic acid, eluent B: water+0.1% formic acid, gradient: 0.0 min 10% A→4 min 90% A→6 min 90% A.

Method 3 (LC-MS): Instrument: Waters Alliance 2790 LC; column: Symmetry C18, 50mm×2.1 mm, 3.5 μm; eluent A: water+0.1% formic acid, eluent B: acetonitrile+0.1% formic acid; gradient: 0.0 min 5% B→5.0 min 10% B→6.0 min 10% B; temperature: 50° C.; flow rate: 1.0 ml/min; UV detection: 210 nm.

Method 4 (LC-MS): ZMD Waters; column: Inertsil ODS3 50 mm×2.1 mm, 3 μm; temperature: 40° C.; flow rate: 0.5 ml/min; eluent A: water+0.05% formic acid, eluent B: acetonitrile+0.05% formic acid, gradient: 0.0 min 5% B→12 min→100% B→15 min 100% B.

Method 5 (LC-MS): MAT 900, Finnigan MAT, Bremen; column: X-terra 50 mm×2.1 mm, 2.5 μm; temperature: 25° C.; flow rate: 0.5 ml/min; eluent A: water+0.01% formic acid, eluent B: acetonitrile+0.01% formic acid, gradient: 0.0 min 10% B→15 min→90% B→30 min 90% B.

Method 6 (LC-MS): TSQ 7000, Finnigan MAT, Bremen; column: Inertsil ODS3 50 mm×2.1 mm, 3 μm; temperature: 25° C.; flow rate: 0.5 ml/min; eluent A: water+0.05% formic acid, eluent B: acetonitrile+0.05% formic acid, gradient: 0.0 min 15% B→15 min→100% B→30 min 100% B.

Method 7 (LC-MS): 7 Tesla Apex II with external electrospray ion source, Bruker Daltronics; column: X-terra C18 50 mm×2.1 mm, 2.5 μm; temperature: 25° C.; flow rate: 0.5 m/min; eluent A: water+0.1% formic acid, eluent B: acetonitrile+0.1% formic acid, gradient: 0.0 min 5% B→13 min→100% B→15 min 100% B.

Method 8 (LC-MS): MS instrument type: Micromass ZQ; HPLC instrument type: Waters Alliance 2795; column: Merck Chromolith SpeedROD RP-18e 50×4.6 mm; eluent A: water+500 μl of 50% formic acid/l; eluent B: acetonitrile+500 μl of 50% formic acid/l; gradient: 0.0 min 10% B→2.0 min 95% B→4.0 min 95% B; oven: 35° C.; flow rate: 0.0 min 1.0 ml/min→2.0 min 3.0 ml/min→4.0 min 3.0 m/min; UV detection: 210 nm.

Method 9 (LC-MS): Instrument: Micromass Platform LCZ with HPLC Agilent series 1100; column: Grom-SIL120 ODS-4 HE, 50 mm×2.0 mm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 m/min; UV detection: 210 nm.

Method 10 (LC-MS): MS instrument type: Micromass ZQ; HPLC instrument type: Waters Alliance 2795; column: Merck Chromolith SpeedROD RP-18e 50×4.6 mm; eluent A: water+500 μl of 50% formic acid/l; eluent B: acetonitrile+500 μl of 50% formic acid/l; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

Method 11 (LC-MS): MS instrument type: Micromass ZQ; HPLC instrument type: Waters Alliance 2790; column: Uptisphere C 18, 50 mm×2.0 mm, 3.0 μm; eluent B: acetonitrile+0.05% formic acid, eluent A: water+0.05% formic acid; gradient: 0.0 min 5% B→2.0 min 40% B→4.5 min 90% B→5.5 min 90% B; oven: 45° C.; flow rate: 0.0 min 0.75 ml/min→4.5 min 0.75 ml/min→5.5 min 1.25 ml/min; UV detection: 210 nm.

Method 12 (LC-MS): MS instrument type: Micromass ZQ; HPLC instrument type: Waters Alliance 2795; column: Phenomenex Synergi 2μ Hydro-RP Mercury 20×4 mm; eluent A: 1 l of water+0.5 ml of 50% formic acid, eluent B: 1 l of acetonitrile+0.5 ml of 50% formic acid; gradient: 0.0 min 90% A (flow rate: 1 ml/min)→2.5 min 30% A (flow rate: 2 ml/min)→3.0 min 5% A (flow rate: 2 ml/min)→4.5 min 5% A (flow rate: 2 mlmin); oven: 50° C.; UV detection: 210 nm.

Method 13 (LC-MS): MS instrument type: Micromass ZQ; HPLC instrument type: HP 1100 series; UV DAD; column: Grom-Sil 120 ODS-4 HE 50×2 mm, 3.0 μm; eluent A: water+500 μl of 50% formic acid/l, eluent B: acetonitrile+500 μl of 50% formic acid/l; gradient: 0.0 min 70% B→4.5 min 90% B; oven: 50° C., flow rate: 0.8 ml/min, UV detection: 210 nm.

Method 14 (LC-MS): Instrument: Micromass Quattro LCZ, with HPLC Agilent series 1100; column: Grom-SIL120 ODS-4 HE, 50 mm×2.0 mm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 nm.

Method 15 (LC-MS): MS Instrument type: Micromass ZQ; HPLC instrument type: Waters Alliance 2790; column: Grom-Sil 120 ODS-4 HE 50×2 mm, 3.0 μm; eluent A: water+500 μl of 50% formic acid/l; eluent B: acetonitrile+500 μl of 50% formic acid/l; gradient: 0.0 min 5% B→2.0 min 40% B=4.5 min 90% B→5.5 min 90% B; oven: 45° C.; flow rate: 0.0 min 0.75 ml/min→4.5 min 0.75 ml 5.5 min→5.5 min 1.25 ml; UV detection: 210 nm.

Method 16 (HPLC): Instrument: HP 1100 with DA detection; column: Kromasil RP-18, 60 mm×2 mm, 3.5 μm; eluent A: 5 ml of perchioric acid/l of water, eluent B: acetonitrile; gradient: 0 min 2% B, 0.5 min 2% B, 4.5 min 90% B, 15 min 90% B; flow rate: 0.75 ml/min; oven: 30° C.; UV detection: 210 nm.

Method 17 (LC-MS): MS instrument type: Micromass ZQ; HPLC instrument type: HP 1100 series; UV DAD; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 rnm.

Method 18 (LC-MS): Instrument: Micromass Platform LCZ with HPLC Agilent series 1100; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C;; WTV detection: 210 mnm.

Method 19 (LC-MS): Instrument: Micromass Quattro LCZ with HPLC Agilent series 1100; column: Phenomenex Synergi 2μ Hydro-RP Mercury 20 mm×4 mm; eluent A: 1 l of water+0.5 ml of 50% formic acid, eluent B: 1 l of acetonitrile+0.5 ml of 50% formic acid; gradient: 0.0 min 90% A→2.5 min 30% A→3.0 min 5% A→4.5 min 5% A; flow rate: 0.0 min 1 m/min, 2.5 min/3.0 min/4.5 min 2 ml/min; oven: 50° C.; UV detection: 208-400 nm.

Method 20 (LC-MS): Instrument: Micromass Platform LCZ with HPLC Agilent series 1100; column: ThermoHypersil-Keystone HyPurity Aquastar, 50 mm×2.1 mm, 3 μm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 ml/min; UW detection: 210 nm.

Method 21 (preparative HPLC/RP-HPLC): column: RP18 Phenomenex Luna C18(2) (New Column), 250 mm×21.2 mm, 5 μm (from Phenomenex, Aschaffenburg, Germany), eluent: acetonitrile-water gradient with addition of 0.2% diethylamine.

Method 22 (HPLC): Instrument: HP 1100 with DA detection; column: Kromasil RP-18, 60 mm×2 mm, 3.5 μm; eluent A: 5 ml of perchloric acid/l of water, eluent B: acetonitrile; gradient: 0 min 2% B, 0.5 min 2% B, 4.5 min 90% B, 9 min 90% B; flow rate: 0.75 ml/min; oven: 30° C.; UV detection: 210 nm.

### CHEMICAL SYNTHESIS OF THE EXAMPLES

Synthesis of the Starting Compounds:

Synthesis of substituted phenylalanine derivatives with (−)-3-(2-benzyloxy-5-iodophenyl)-2(S)-tert-butoxycarbonylaminopropionic acid [(−)-6A] as example

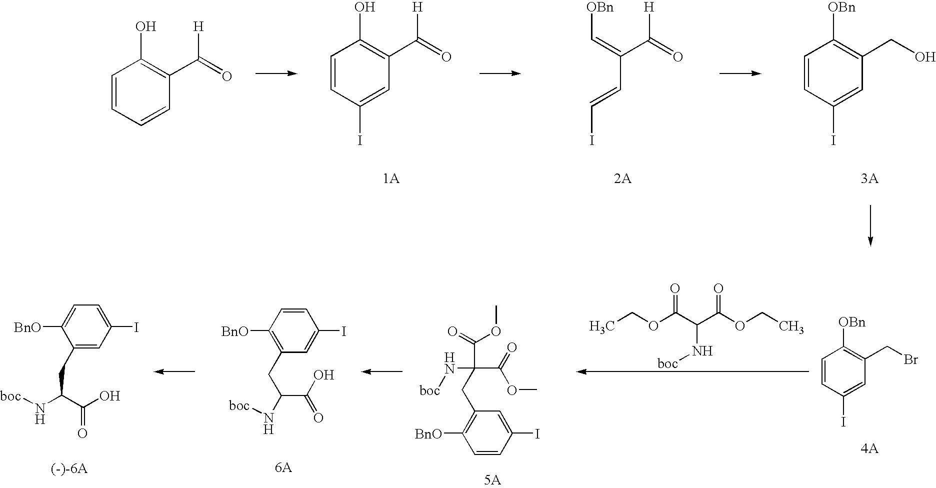

Synthesis of protected hydroxyomithine derivatives with 5-benzyloxycarbonylamino-2(S)-tert-butoxycarbonylamino-4(R)-(tert-butyl-dimethylsilyloxy)pentanoic acid (14A) as example

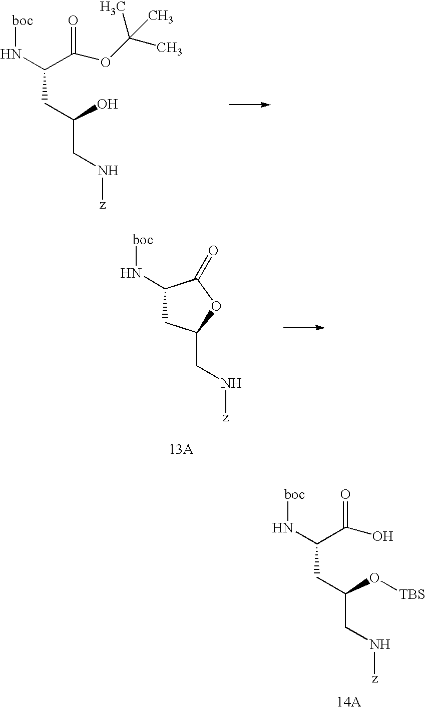

Synthesis of substituted phenylalanine derivatives with methyl 2-(benzyloxy)-N-[(benzyloxy)carbonyl]-5-bromo-L-phenylalaninate] (56A) as example

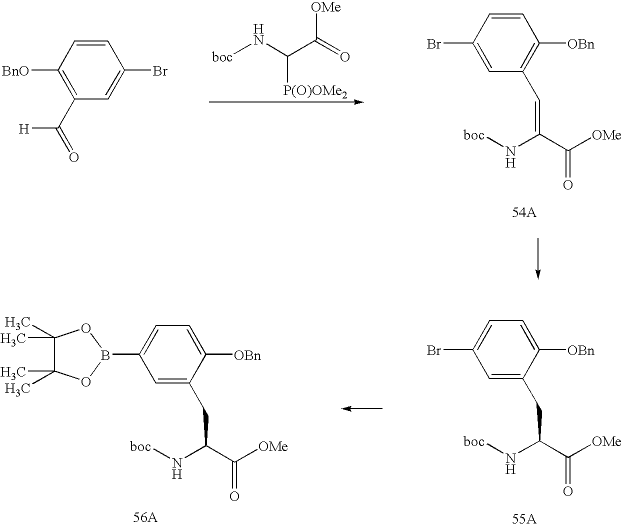

Synthesis of protected biphenyl-bisamino acids with 2(S)-trimethylsilanylethyl 2(S)-benzyloxycarbonylamino-3-[4,4′-bisbenzyloxy-3′-(2(S)-benzyloxycarbonyl-2(S)-tert-butoxycarbonylaminoethyl)biphenyl-3-yl]propionate (12A) as example

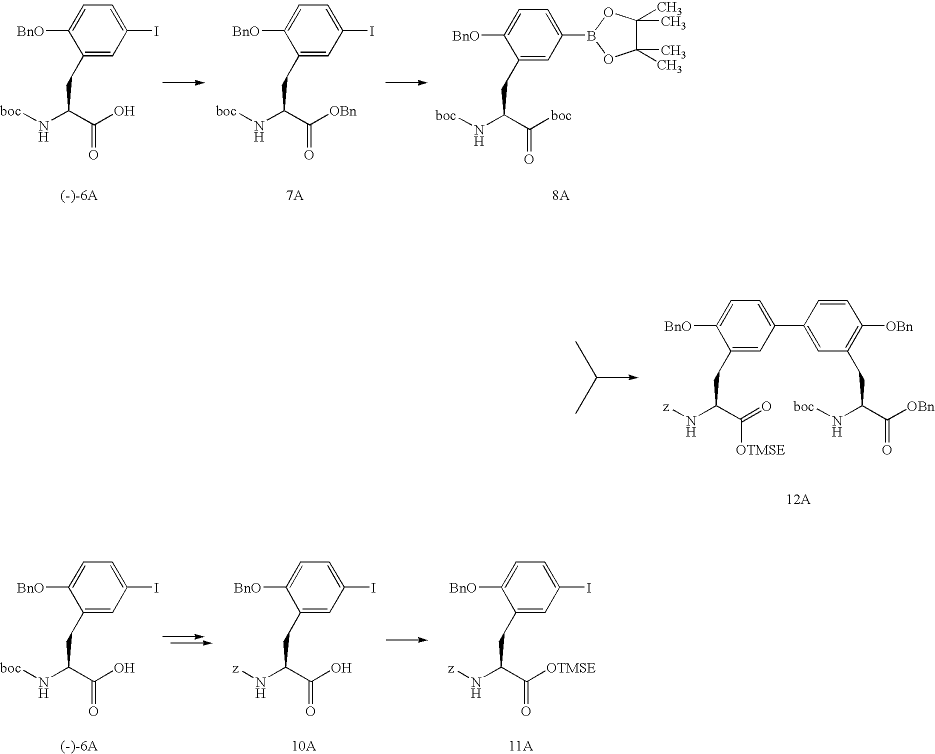

Cyclization of the biphenyl-bisamino acids 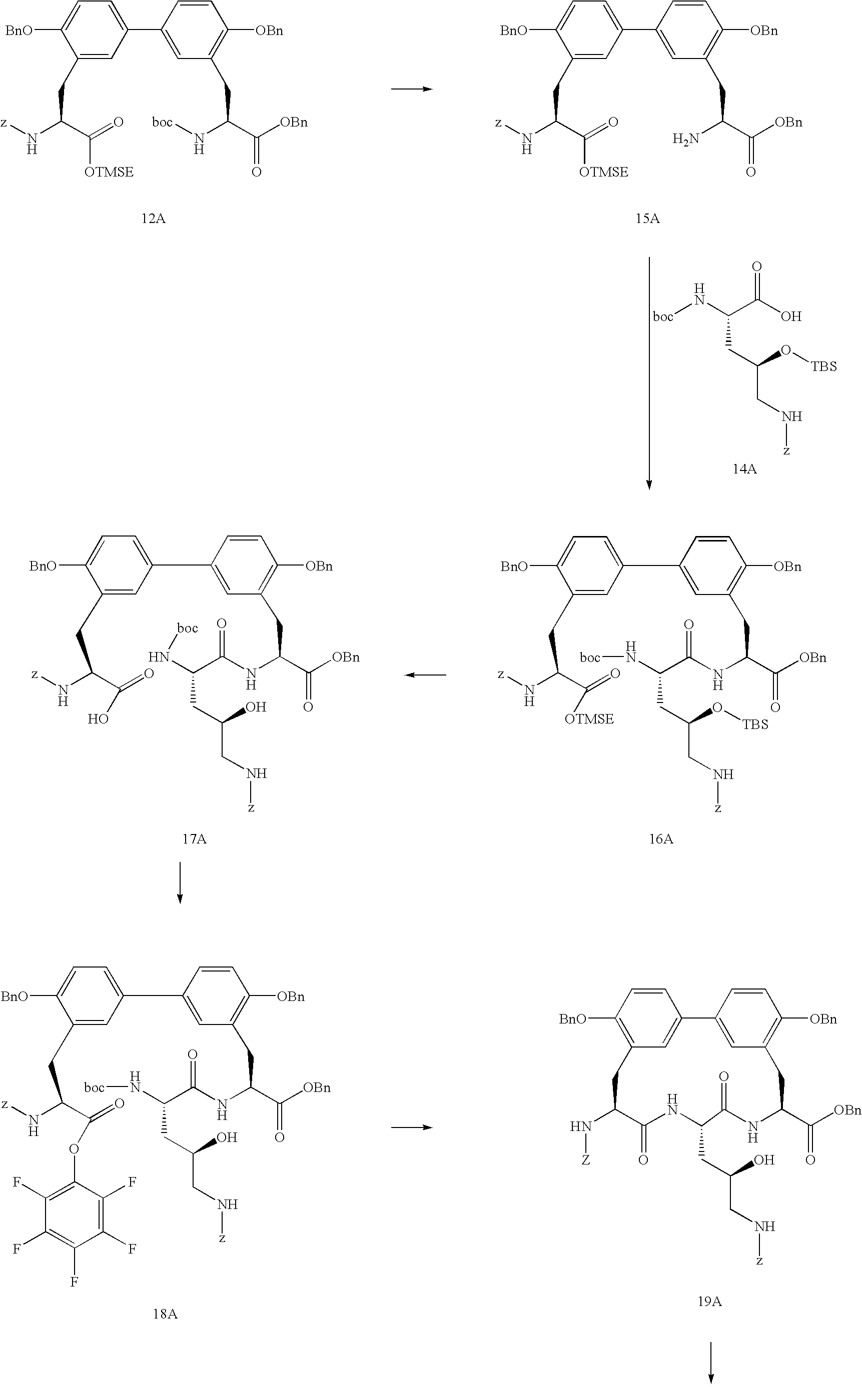 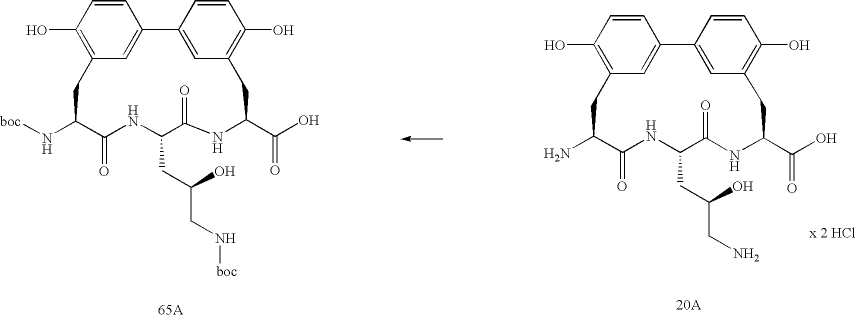
Starting Compounds

### Example 1A

### 2-Hydroxy5-iodobenzaldehyde

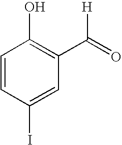

A solution of 250 g (1.54 mol) of iodine chloride in 600 ml of anhydrous dichloromethane is added dropwise over the course of 2 h to a solution of 188 g (1.54 mol) of salicylaldehyde in 1 l of anhydrous dichloromethane in a heat-dried flask under argon. After stirring at RT for 3 days, a saturated aqueous sodium sulphite solution is added with vigorous stirring. The organic phase is separated off, washed once with water and saturated aqueous sodium chloride solution and dried over sodium sulphate. The solvent is evaporated and the residue is recrystallized from ethyl acetate. 216 g (57% of theory) of the product are obtained.

LC-MS (ESL Method 4): m/z=246 (M−H)⁻.

¹H-NMR (400 MHz, CDCl₃): δ=6.7 (d, 1H), 7.77 (dd, 1H), 7.85 (d, 1H), 9.83 (s, 1H), 10.95 (s, 1H).

### Example 2A

### 2-Benzyloxy-5-iodobenzaldehyde

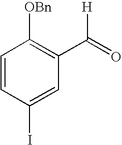

67.2 g (0.48 mol) of potassium carbonate are added to a solution of 100 g (0.40 mol) of 2-hydroxy-5-iodobenzaldehyde (Example 1A) in 1.5 l of dimethylformamide and, after a few minutes, 51 ml (0.44 mol) of benzyl chloride are added. The reaction mixture is stirred under reflux at 120° C. for 24 h. After stirring at RT for a further 24 h and addition of 1.5 l of water, a solid crystallizes out. The precipitate is filtered off with suction, washed twice with water and dried in vacuo. The solid is recrystallized from 230 ml of ethanol. 122.9 g (90% of theory) of the product are obtained.

LC-MS (ESI, Method 4): m/z=338 M+H)⁺.

¹H-NMR (400 MHz, CDCl₃): δ=5.18 (s, 2H), 6.84 (d, 1H), 7.33-7.45 (m, 5H), 7.78 (dd, 1H), 8.12 (d, 1H), 10.4 (s, 1H).

### Example 3A

### (2-Benzyloxy-5-iodophenyl)methanol

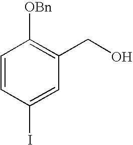

100 ml of a 1M diisobutylaluminium hydride solution in dichloromethane are added to a solution, cooled to 0° C., of 33.98 g (100.5 mmol) of 2-benzyloxy-5-iodobenzaldehyde (Example 2A) in 200 ml of dichloromethane. After stirring at 0° C. for 2 h, a saturated potassium sodium tartrate solution is added while cooling (highly exothermic reaction), and the reaction mixture is stirred for a further 2 h. After separation of the phases, the organic phase is washed twice with water and once with saturated aqueous sodium chloride solution and dried over sodium sulphate. The solvent is evaporated off in vacuo. 31.8 g (93% of theory) of the product are obtained.

¹H-NMR (400 MHz, CDCl₃): δ=2.17 (t, 1H), 4.68 (d, 2H), 5.1 (s, 2H), 6.72 (d, 1H), 7.32-7.42 (m, 5H), 7.54 (dd, 1), 7.63 (d, 1H).

### Example 4A

### 1-Benzyloxy-2-bromoethyl-4-iodobenzene

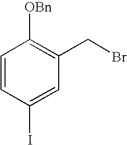

3.3 ml (35 mmol) of phosphorus tribromide are added dropwise to a solution of 35 g (103 mmol) of (2-benzyloxy-5-iodophenyl)methanol (Example 3A) in 350 ml of toluene at 40° C. The temperature of the reaction mixture is raised to 100° C. over the course of 15 min and is stirred at this temperature for a further 10 min. After cooling, the two phases are separated. The organic phase is washed twice with distilled water and once with saturated aqueous sodium chloride solution. The organic phase is dried over sodium sulphate and evaporated. The yield amounts to 41 g (99% of theory).

¹H-NMR (300 MHz, CDCl₃): δ=4.45 (s, 2H), 5.06 (s, 2H), 7.30 (m, 8H).

### Example 5A

### Diethyl 2-(2-benzyloxy-5-iodobenzyl)-2-tert-butoxycarbonylaminomalonate

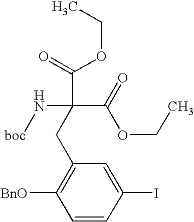

41 g (101.7 mmol) of 1-benzyloxy-2-bromomethyl4-iodobenzene (Example 4A) are added to a solution of 28 g (101.7 mmol) of diethyl 2-[N-(tert-butoxycarbonyl)amino]malonate and 7.9 ml (101.7 mmol) of sodium ethoxide in 300 ml of ethanol. After stirring at RT for 3 h, the precipitated product is filtered off with suction. After drying in vacuo, 55 g (90% of theory) of product are isolated.

¹H-NMR (400 MHz, CDCl₃): δ=1.12 (t, 6 H), 1.46 (s, 9H), 3.68 (s, 2H), 3.8-3.9 (m, 2H), 4.15-4.25 (m, 2H), 5.0 (s, 2H), 5.7 (s, 1H), 6.58 (d, 1H), 7.28-7.4 (m, 6H), 7.4 (dd, 1H).

### Example 6A

### 5 (±)-3-(2-Benzyloxy-5-iodophenyl)-2-tert-butoxycarbonylaminopropionic acid

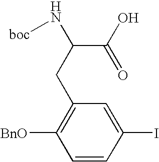

400 ml of 1N sodium hydroxide solution are added to a suspension of 58 g (97 mmol) of diethyl 2-(2-benzyloxy-5-iodobenzyl)-2-tert-butoxycarbonyl-aminomalonate (Example 5A) in 800 ml of a mixture of ethanol and water (7:3). After 3 h under reflux and after cooling to room temperature, the pH of the reaction mixture is adjusted to about pH 2 with conc. hydrochloric acid. The reaction mixture is evaporated. The residue is taken up in MTBE and water. The aqueous phase is extracted three times with MTBE. The combined organic phases are dried over sodium sulphate, filtered and concentrated. Drying in vacuo results in 47 g (97% of theory) of the product.

¹H-NMR (400 MHz, DMSO-d₆): δ=1.32 (s, 9H), 2.68 (dd, 1H), 3.18 (dd, 1H), 4.25 (m, 1H), 5.15 (s, 2H), 6.88 (d, 1H), 7.08 (d, 1H), 7.30-7.40 (m, 3 H), 7.45-7.55 (m, 3 H).

### Example (−)6A

### 3-(2-Benzyloxy-5-iodophenyl)-2(S)-tert-butoxycarbonylaminopropionic acid

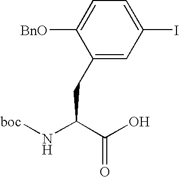

The racemate from Example 6A [(±)-3-(2-benzyloxy-5-iodophenyl)-2(S)-tert-butoxycarbonylaminopropionic acid] is separated on a chiral stationary silica gel phase based on the selector from poly(N-methacryloyl-L-leucine dicyclopropylmethylamide) using an i-hexane/ethyl acetate mixture as eluent. The enantiomer eluted first (98.9% ee) is dextrorotatory in dichloromethane ([α]²¹_{D}: +3.00, c=0.54, dichloromethane) and corresponds to the (R) enantiomer Example (+)-6A, as was determined by single-crystal X-ray structural analysis. The purity of the second, levorotatory enantiomer Example (−)-6A, i.e. the (S) enantiomer, is >99% ee.

### Example 7A

### Benzyl 3-(2-benzyloxy-5-iodophenyl)-2(S)-tert-butoxycarbonylaminopropionate

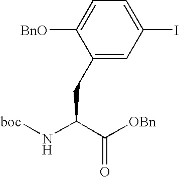

Under argon, 10 g (20.11 mmol) of (−)-3-(2-benzyloxy-5-iodophenyl)-2(S)-tert-butoxycarbonylaminopropionic acid (Example (−)-6A) are dissolved in 200 ml acetonitrile. To this are added 246 mg (2.01 mmol) of 4-dimethylaminopyridine and 4.16 ml (40.22 mmol) of benzyl alcohol. The mixture is cooled to −10° C., and 4.63 g (24.13 mmol) of EDC are added. The mixture is allowed slowly to reach RT and is stirred overnight. After about 16 h, the mixture is concentrated in vacuo, and the residue is purified by column chromatography on silica gel (mobile phase: dichloromethane). Yield: 10.65 g (88% of theory).

HPLC (Method 1): Rₜ=6.03 min; LC-MS (Method 3): Rₜ=4.70 min l

MS (DCI): m/z=605 (M+NH₄)⁺.

¹H-NMR (200 MHz, CDCl₃): δ=1.38 (s, 9H),2.97 (dd, 1H), 3.12 (dd, 1H), 4.50-4.70 (m, 1H), 5.00-5.10 (m, 4H, 5.22 (d, 1H), 6.64 (d, 1H), 7.28-7.36 (n, 7H), 7.37-7.52 (m, 5H).

### Example 8A

### Benzyl 3-[2-benzyloxy-5-(4,4,5,5-tetramethyl[1,3,2]dioxaborolan-2-yl)phenyl]-2(S)-tert-butoxycarbonylaminopropionate

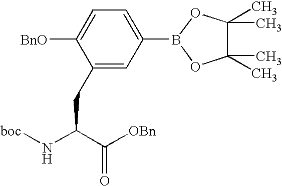

5.15g (52.60 mmol) of potassium acetate are added to a solution of 10.30 g (17.53 mol) of benzyl 3-(2-benzyloxy-5-iodophenyl)-2(S)-tert-butoxycarbonyl-aminopropionate (Example 7A) in 70 ml of DMSO. The mixture is deoxygenated by passing argon through the vigorously stirred solution for 15 min. Then 5.17 g (20.16 mmol) of 4,4,4′,4′,5,5,5′,5′-octamethyl-2,2′-bi-1,3,2-dioxaborolane and 515 mg (0.70 mmol) of bis(diphenylphosphino)ferrocenepalladium(II) chloride are added. The mixture is then heated to 80° C. under a gentle stream of argon and after 6 h is cooled again. The mixture is filtered through silica gel (mobile phase: dichloromethane). The residue is purified by column chromatography on silica gel (mobile phase: cyclohexane:ethyl acetate 4:1).

Yield: 8.15 g (79% oftheory)

HPLC (Method 1): Rₜ=6.26 min

LC-MS (Method 2): Rₜ=5.93 and 6.09 min

MS (ED: m/z=588 (M+H)⁺

¹H-NMR (200 Mob; CDCl₃): δ=1.26 (s, 6H), 1.33 (s, 9H), 1.36 (s, 6H), 2.91-3.10 (m, 1H), 3.12-3.28 (m, 1H), 4.49-4.68 (m, 1H), 5.05 (dd, 2H), 5.11 (dd, 2H), 5.30 (d, 1H), 6.90 (d, 1H), 7.27-7.37 (m, 7H), 7.38-7.42 (m, 3), 7.55-7.62 (m, 1H), 7.67 (dd, 1H).

### Example 9A

### 2(S)-Amino-3-(2-benzyloxy-5-iodophenyl)propionic acid hydrochloride

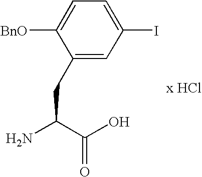

12 g (24.13 mmol) of 3-(2-benzyloxy-5-iodophenyl)-2(S)-tert-butoxycarbonylamino-propionic acid (Example (−)-6A) are put under argon into 60 ml of a 4M hydrochloric acid solution in dioxane and stirred at RT for 2 h. The reaction solution is concentrated and dried under high vacuum.

Yield: 10.47 g (100% of theory)

HPLC (Method 1): Rₜ=4.10 min

MS (EI): m/z=398 (M+H+HCl)⁺

¹H-NMR (200 Mz, CDCl₃): δ=3.17-3.31 (m, 1H), 3.33-3.47 (m, 1H), 4.22 (t, 1H), 5.13 (s, 2H), 6.69 (d, 1 H), 7.24-7.40 (m, 2H), 7.41-7.45 (m, 2H), 7.48 (d, 1H), 7.52 (d, 1H), 7.60 (d, 1H), 8.66 (br.s, 2H).

### Example 10A

### 2(S)-Benzyloxycarbonylamino-3-(2-benzyloxy-5-iodophenyl)propionic acid

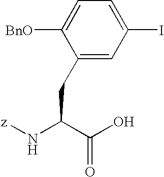

9.25 ml (53.09 mol) of N,N-diisopropylethylamine are added to a solution of 10.46 g (24.13 mmol) of 2(S)-amino-3-(2-benzyloxy-5-iodophenyl)propionic acid hydrochloride (Example 9A) in DMF. 6.615 g (26.54 mmol) of N-(benzyloxycarbonyl)succinimide (Z-OSuc) are added thereto. The resulting solution is stirred overnight and then evaporated in vacuo. The residue is taken up in dichloromethane and extracted twice each with 0.1N hydrochloric acid and saturated aqueous sodium chloride solution. The organic phase is dried, filtered and concentrated. The mixture is purified by column chromatography on silica gel (mobile phase: cyclohexane/diethyl ether 9:1 to 8:2).

Yield: 8.30 g (65% of theory)

HPLC (Method 1): Rₜ=5.01 min

MS (EI): m/z=532 (M+H)⁺

¹H-NMR (200 MHz, DMSO-d₆): δ=3.14-3.3 (m, 2 H), 4.25-4.45 (m, 1H), 4.97 (s, 2H), 5.14 (s, 2H), 6.88 (d, 1 H), 7.20-7.56 (m, 12 H), 7.62 (d, 1 H), 12.73 (br.s, 1H).

### Example 11A

### (2-Trimethylsilyl)ethyl 2(S)-benzyloxycarbonylamino-3-(2-benzyloxy-5-iodophenyl)propionate

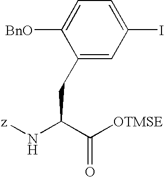

8.35 g (15.7 mmol) of 2(S)-benzyloxycarbonylamino-3-(2-benzyloxy-5-iodophenyl)propionic acid (Example 10A) are introduced into 150 ml of THF, and 2.14 g (18.07 mmol) of 2-trimethylsilylethanol and 250 mg (2.04 mmol) of 4-dimethylaminopyridine are added. The mixture is cooled to 0° C., and 2.38 g (2.95 ml, 18.86 mmol) of N,N′-diisopropylcarbodiimide dissolved in 40 ml of THF are added. The mixture is stirred at RT overnight and evaporated in vacuo for working up. The residue is taken up in dichloromethane and extracted twice each with 0.1N hydrochloric acid and saturated aqueous sodium chloride solution. The organic phase is dried, filtered and concentrated. The mixture is purified by column chromatography (silica gel, mobile phase: cyclohexane/diethyl ether 9:1 to 8:2).

Yield: 8.2 g (83% of theory)

HPLC (Method 1): Rₜ=6.42 min

MS (EI): m/z=532 (M+H)⁺

¹H-NMR (300 MHz, CDCl₃): δ=0.01 (s, 9H), 0.88 (t, 2H), 2.96 (dd, 1H), 3.13 (dd, 1H), 4.04-4.17 (m, 2H), 4.51-4.62 (n, 1H), 4.95-5.05 (m, 4H), 5.44 (d, 1H), 6.64 (d, 1H), 7.25-7.33 (m, 7 H), 7.37 (dd, 4H), 7.45 (dd, 1H).

### Example 12A

### 2-(Trimethylsilyl)ethyl 2(S)-benzyloxycarbonylamino-3-[4,4′-bisbenzyloxy-3′-(2(S)-benzyloxycarbonyl-2-tert-butoxycarbonylaminoethyl)biphenyl-3-yl]propionate

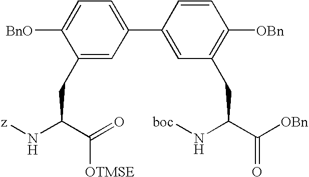

45.8 mg (0.05 mmol) of bis(diphenylphosphino)ferrocenepalladium(II) chloride (PdCl₂(dppf)) and 0.325 g (1.0 mmol) of caesium carbonate are added to a solution of 0.316 g (0.5 mmol) of (2-trimethylsilyl)ethyl 2(S)-benzyloxycarbonylamino-3-(2-benzyloxy-5-iodophenyl)propionate (Example 11A) in 2.5 ml of degassed DMF under argon at RT. The reaction mixture is heated to 40° C. Over the course of 30 min, a solution of 0.294 g (0.5 mmol) of benzyl 3-[2-benzyloxy-5-(4,4,5,5-tetramethyl-[1,3,2]dioxaborolan-2-yl)phenyl]-2(S)-tert-butoxycarbonylamino-propionate (Example 8A) in 2.5 ml of degassed DMF is added dropwise. The reaction mixture is stirred at 40° C. for 4 h and at 50° C. for a further 2 h. The solvent is evaporated and the residue is taken up in ethyl acetate. The organic phase is extracted twice with water, dried over sodium sulphate and concentrated. The crude product is purified by chromatography on silica gel with dichloromethane/ethyl acetate (30/1). 0.320 g (66% of theory) of the product is obtained.

HPLC (Method 1): Rₜ=7.65 min

MS (EI): m/z=987 (M+Na), 965 (M+H)⁺

¹H-NMR (200 MHz, CDCl₃): δ=0.00 (s, 9H), 0.90 (t 2H), 1.37 (s, 9H), 3.02-3.35 (m, 4H) 4.06-4.25 (n, 2H), 4.55-4.73 (m, 2H), 4.98-5.18 (m, 8H), 5.40 (d, 1H), 5.63 (d, 1H), 6.88-7.00 (m, 2H), 7.19-7.39 (m, 20H), 7.42-7.53 (m, 4H).

### Example 13A

### Benzyl ({(2R,4S)4-[(tert-butoxycarbonyl)amino]-5-oxotetrahydrofuran-2-yl}-methyl)carbamate

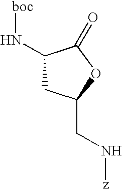

A solution of 7.60 g (17.3 mmol) of tert-butyl 5-benzyloxycarbonylamino-2(S)-tert-butoxycarbonylamino-4(R)-hydroxypentanoate (Org. Lett., 2001, 3, 20, 3153-3155) in 516 ml of dichloromethane and 516 ml of trifluoroacetic acid is stirred at RT for 2 h. The solvent is evaporated. The remaining crude product is dissolved in 2.6 l of anhydrous methanol and, while stirring at 0° C., 6.3 g (28.8 mmol) of di-tert-butyl dicarbonate and 7.3 ml (52.43 mmol) of triethylamine are added. After 15 h, the reaction solution is evaporated and the residue is taken up in 1 l of ethyl acetate. After the phases have been separated, the organic phase is extracted twice with a 5% strength citric acid solution, twice with water and once with saturated aqueous sodium chloride solution, dried over sodium sulphate and concentrated. The crude product is purified by chromatography on silica gel with toluene/acetone (5/1). 4.92 g (78% of theory) of the product are obtained.

| | |
|---|---|
| LC-HR-FT-ICR-MS | calc. for C₁₈H₂₈N₃O₆ (M + NH₄)⁺ 382.19726 |
| (Method 7): | found 382.19703 |

¹H-NMR (400 MHz, CDCl₃): δ=1.45 (s, 9H), 2.3-2.4 (m, 1H), 2.45-2.55 (m, 1H), 3.3-3.4 (m, 1H), 3.5-3.6 (m, 1H), 4.17-4.28 (m, 1H), 4.7-4.8 (m, 1H), 5.0-5.15 (m, 4H), 7.3-7.4 (m, 5H).

### Example 14A

### 5-Benzyloxycarbonylamino-2(S)-tert-butoxycarbonylamino-4(R)-(tert-butyldimethylsilanyloxy)pentanoic acid

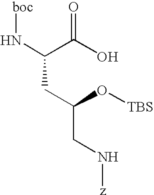 Method A:

2 ml of 1M sodium hydroxide solution are added to a solution of 0.73 g (2 mmol) of from Example 13A in 50 ml of 1,4-dioxane at 0° C. The reaction solution is stirred for 2 h and then evaporated. The residue is taken up in 50 ml of dichloromethane. 1.12 ml (8 mmol) of triethylamine are added to this solution and, after a short time, 1.38 ml (6 mmol) of tert-butyldimethylsilyl trifluoromethanesulphonate are added dropwise. After stirring at RT for 3 h, the reaction mixture is diluted with dichloromethane. The organic phase is washed with 1N sodium bicarbonate solution, dried over sodium sulphate and evaporated. The crude product is dissolved in 7.4 ml of 1,4-dioxane, and 36.2 ml of 0.1N sodium hydroxide solution are added. After stirring at RT for 3 h, the reaction solution is evaporated, and the residue is taken up in water and ethyl acetate. The organic phase is extracted three times with ethyl acetate. The combined organic phases are dried over sodium sulphate and evaporated. 0.90 g (90% of theory) of the product is obtained.

Method B:

A solution of 14.0 g (38 mmol) of benzyl 2(S)-tert-butoxycarbonylamino-4(R)-hydroxy-5-nitropentanoate (Example 22A) in 840 ml of ethanol/water 9/1 is mixed with 1.96 g of palladium on carbon (10%) and hydrogenated under atmospheric pressure at RT for 24 h. The mixture is filtered through kieselguhr, and the filtrate is mixed with 14.7 g (114 mmol) of diisopropylethylamine. Then 11.4 g (45.6 mmol) of N-(benzyloxycarbonyloxy)succinimide are added, and the mixture is stirred at RT for 4 h. The solution is concentrated, and the residue is taken up in dichloromethane and extracted twice with 0.1N hydrochloric acid. The organic phase is separated off and made alkaline with 14.7 g (114 mmol) of diisopropylamine. The solution is cooled to 0° C., 30.1 g (114 mmol) of dimethyl-tert-butylsilyl trifluoromethanesulphonate are added, and the mixture is stirred at RT for 2.5 h. The organic phase is washed with saturated sodium bicarbonate solution, dried over sodium sulphate and evaporated. The residue is dissolved in 50 ml of dioxane, mixed with 200 ml of 0.1N sodium hydroxide solution and stirred at RT for 3 h. After extraction several times with ethyl acetate, the collected organic phases are dried over sodium sulphate and concentrated in vacuo. The residue is chromatographed on silica gel (mobile phase: dichloromethane/ethanol 20/1, 9/1). 8.11 g (43% of theory) of the product are obtained.

MS (ESI): m/z=497 (M+H)⁺

¹H-NMR (300 MHz, DMSO-d6): δ=0.00 (s, 6H), 0.99 (s, 9H), 1.33 (s, 9H), 1.59 (m, 1H), 1.80 (m, 1H), 2.75-3.15 (m, 2H), 3.81 (m, 1H), 3.98 (m, 1H), 4.96 (m, 2H), 7.04 (d, 1H), 7.19 (m, 1H), 7.30 (m, 5H), 12.37 (br. s, 1H).

### Example 15A

### 2-(Trimethylsilyl)ethyl 3-[3′-2(S)-amino-2-benzyloxycarbonylethyl)-4,4′-bisbenzyloxybiphenyl-3-yl]-2(S)-benzyloxycarbonylaminopropionate hydrochloride

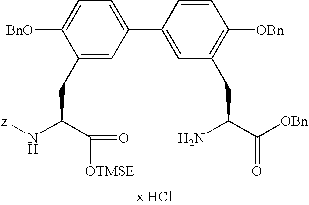

50 ml of a 4M hydrochloric acid/dioxane solution are added over the course of about 20 min to a solution, cooled to 0° C., of 2.65 g (2.75 mmol) of 2-(trimethylsilyl)ethyl 2(S)-benzyloxycarbonylamino-3-[4,4′-bisbenzyloxy-3′-(2(S)-benzyloxycarbonyl-2-tert-butoxycarbonylaminoethyl)biphenyl-3-yl]propionate (Example 12A) in 50 ml of anhydrous dioxane. After stirring for 3 h, the reaction solution is evaporated and dried under high vacuum.

Yield: 100% of theory

HPLC (Method 1): Rₜ=5.96 min

MS (EI): m/z=865 (M+H)⁺

### Example 16A

### Benzyl 2(S)-[5-benzyloxycarbonylamino-2(S)-tert-butoxycarbonylamino-4(R)-(tert-butyldimethylsilyloxy)pentanoylamino]-3-{4,4′-bisbenzyloxy-3′-[2(S)-benzyloxycarbonylamino-2-(2-trimethylsilylethoxycarbonyl)ethyl]biphenyl-3-yl}propionate

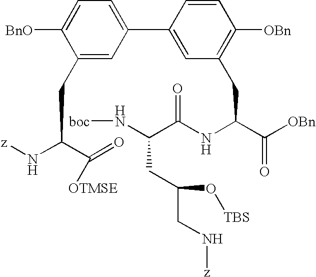

0.219 g (0.58 mmol) of HATU and 0.082 g (0.63 mmol) of N,N-diisopropylethylamine are added to a solution, cooled to 0° C., of 0.520 g (0.58 mmol) of (2-trimethylsilyl)ethyl 3-[3′-(2(S)-amino-2-benzyloxycarbonylethyl)4,4′-bisbenzyloxybiphenyl-3-yl]-2(S)-benzyloxycarbonylaminopropionate hydrochloride (Example 15A) and 0.287 g (0.58 mmol) of 5-benzyloxycarbonylamino-2(S)-tert-butoxycarbonylamino-4-(R)-(tert-butyldimethylsilyloxy)pentanoic acid (Example 14A) in 7.3 ml of anhydrous DMF. After stirring at 0° C. for 30 min, an additional 0.164 g (1.26 mmol) of N,N-diisopropylethylamine is added. The reaction mixture is stirred at RT for 15 h. The solvent is then evaporated, and the residue is taken up in ethyl acetate. The organic phase is washed three times with water and once with saturated aqueous sodium chloride solution, dried over sodium sulphate and concentrated. The crude product is purified by chromatography on silica gel with dichloromethane/ethyl acetate (gradient 30/1→20/1→10/1). 533 mg (66% of theory) of the product are obtained.

LC-MS (ESI, Method 6): m/z=1342-(M+H)⁺, 1365 (M+Na)⁺

### Example 17A

### 2(S)-Benzyloxycarbonylamino-3-{4,4′-bisbenzyloxy-3′-[2(S)-benzyloxycarbonyl-2-(5-benzyloxycarbonylamino-2(S)-tert-butoxycarbonylamino-4(R)-hydroxypentanoylamino)ethyl]biphenyl-3-yl}propionic acid

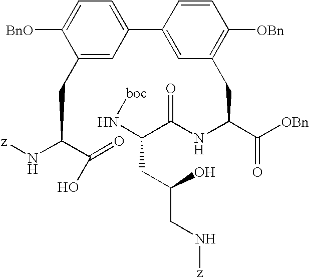

1.8 ml of 1N tetrabutylammonium fluoride in THF are added dropwise to a solution of 800 mg (0.6 mmol) of benzyl 2(S)-[5-benzyloxycarbonylamino-2(S)-tert-butoxycarbonylamino-4(R)-(tert-butyldimethylsilyloxy)pentanoylamino]-3-{4,4′-bisbenzyloxy-3′-[2(S)-benzyloxycarbonylamino-2-(2-trimethylsilylethoxycarbonyl)-ethyl]biphenyl-3-yl}propionate (Example 16A) in 26 ml of absolute DMF at RT. After 25 min at RT, the mixture is cooled to 0° C. and a large amount of ice-water is added. Ethyl acetate and some IN hydrochloric acid are immediately added; The organic phase is dried with magnesium sulphate, concentrated and dried under high vacuum for 1 h. The crude product is reacted without further purification.

LC-MS (ESI, Method 4): m/z=1129 (M+H)⁺

| | |
|---|---|
| LC-HR-FT-ICR-MS: | calc. C₆₅H₆₉N₄O₁₄ (M + H)⁺ 1129.48048 |
| | found 1129.48123 |

### Example 18A

### Benzyl 2(S)-(5-benzyloxycarbonylamino-2(S)-tert-butoxycarbonylamino-4(R)-hydroxypentanoylamino)-3-[4,4′-bisbenzyloxy-3′-(2(S)-benzyloxycarbonylamino-2-pentafluorophenyloxycarbonylethyl)biphenyl-3-yl]propionate

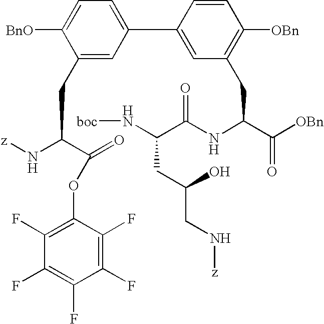

691 mg (crude mixture, approx. 0.6 mmol) of 2(S)-benzyloxycarbonylamino-3-{4,4′-bisbenzyloxy-3′-[2(S)-benzyloxycarbonyl-2-(5-benzyloxycarbonylamino-2(S)-tert-butoxycarbonylamino-4(R)-hydroxypentanoylamino)ethyl]biphenyl-3-yl}propionic acid (Example 17A) are introduced into 25 ml of dichloromethane, and 547.6 mg (2.98 mmol) of pentafluorophenol, dissolved in 6 ml of dichloromethane, are added. 7.3 mg (0.06 mmol) of DMAP are added, and the mixture is cooled to −25° C. (ethanol/carbon dioxide bath). At −25° C., 148 mg (0.774 mmol) of EDC are added. The mixture slowly warms to RT overnight. The reaction mixture is concentrated in vacuo and briefly dried under high vacuum. The crude product is reacted without further purification.

LC-MS (ESI, Method 5): m/z=1317 (M+Na)⁺, 1295 (M+H)⁺

| | |
|---|---|
| LC-HR-FT-ICR-MS: | calc. C₇₁H₆₈F₅N₄O₁₄ (M + H)⁺ 1295.46467 |
| | found 1295.46430 |

### Example 19A

### Benzyl 5,17-bisbenzyloxy-14(S)-benzyloxycarbonylamino-11(S)-(3-benzyloxy-carbonylamino-2(R)-hydroxypropyl)-10,13-dioxo-9,12-diazatricyclo[14.3.1.1^{2,6}]-henicosa-1(19),2,4,6(21),16(20),17-hexaene-8(S)-carboxylate

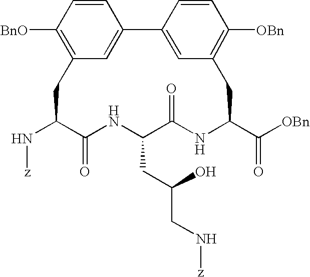 Method A:

4 ml of a 4M hydrochloric acid solution in dioxane are added to a solution of 119.3 mg of benzyl 2(S)-(5-benzyloxycarbonylamino-2(S)-tert-butoxycarbonyl-amino-4(R)-hydroxypentanoylamino)-3-[4,4′-bisbenzyloxy-3′-(2(S)-benzyloxy-carbonylamino-2-pentafluorophenyloxycarbonylethyl)biphenyl-3-yl]propionate (Example 18A) in 2.7 ml of 1,4-dioxane. Until the reaction is complete, a further 1.5 ml of a 4M hydrochloric acid solution in dioxane are added. The reaction solution is evaporated and codistilled with chloroform twice. The crude product (LC-HR-FT-ICR-MS, Method 7: calc. for C₆₆H₆₀F₅N₄O₁₂ (M+H)⁺ 1195.41224, found 1195.41419) is dissolved in 100 ml of chloroform and added dropwise over the course of 3 h to a very efficiently stirred suspension of 200 ml of chloroform and 100 ml of saturated aqueous sodium bicarbonate solution. The reaction mixture is vigorously stirred for 2 h. After the two phases have been separated, the aqueous phase is extracted with chloroform. The combined organic phases are washed with 5% strength aqueous citric acid solution, dried over magnesium sulphate and evaporated to dryness. The crude product is washed with acetonitrile and dried under high vacuum.

Yield: 60.5 mg (65% of theory)

LC-MS (ESI, Method 5): m/z=1011 (M+H)⁺.

Method B:

771 mg (0.595 mmol) of benzyl 2(S)-(5-benzyloxycarbonylamino-2(S)-tert-butoxycarbonylamino-4(R)-hydroxypentanoylamino)-3-[4,4′-bisbenzyloxy-3′-(2(S)-benzyloxycarbonylamino-2-pentafluorophenyloxycarbonylethyl)biphenyl-3-yl]propionate (Example 18A) are dissolved in 8 ml of dioxane and then, at 0° C., 16 ml of a 4N hydrochloric acid solution in dioxane are added dropwise. After 45 min, 6 ml of a 4N hydrochloric acid solution in dioxane are again added, and after 15 min a further 8 ml are added. The mixture is stirred at 0° C. for 30 min before the reaction solution is concentrated under mild conditions, codistilled with chloroform (twice) and briefly dried under high vacuum. The crude product (732 mg, 0.59 mmol) is dissolved in 1000 ml of chloroform, and a solution of 6 ml of triethylamine in 50 ml of chloroform is added dropwise. The mixture is stirred at RT overnight. The mixture is worked up by evaporating under mild conditions in vacuo and stirring the residue in acetonitrile. The resulting crystals are filtered off with suction, washed with acetonitrile and dried under high vacuum.

Yield: 360 mg (60% of theory)

MS (EI): m/z=1011 (M+H)⁺

HPLC (Method 1): Rₜ=5.59 min

¹H-NMR (400 MHz, DMSO-d₆): δ=1.52-1.65 (m, 1H), 1.73-1.84 (m, 1H), 2.82-3.01 (m, 3H), 3.02-3.11 (m, 1H), 3.46 (s, 1H), 3.57-3.68 (m, 1H), 4.47-4.56 (m, 1H), 4.64-4.71 (m, 1H), 4.73-4.85 (m, 2H), 4.88-5.00 (m, 4H), 5.09 (s, 2H), 5.14-5.20 (m, 4H), 6.29 (d, 1H) 7.21-7.40 (m, 20H), 7.41-7.48 (m, 9H), 8.77 (d, 1H), 8.87 (d, 1H).

### Example 20A

### 14(S)-Amino-1(S)-(3-amino-2(R)-hydroxypropyl)-5,17-dihydroxy-10,13-dioxo-9,12-diazatricyclo[14.3.1.1^{2,6}]henicosa-1(19),2,4,6(21),16(20),17-hexaene-8(S)-carboxylic acid dihydrochloride (biphenomycin B)

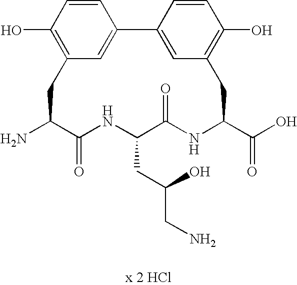

200 mg (0.20 mmol) of benzyl 5,17-bisbenzyloxy-14(S)-benzyloxycarbonylamino-11(S)-(3-benzyloxycarbonylamino-2(R)-hydroxypropyl)-10,13-dioxo-9,12-diazatricyclo[14.3.1.1^{2,6}]henicosa-1(19),2,4,6(21),16(20),17-hexaene-8(S)-carboxylate (Example 19A) are put into 220 ml of an acetic acid/water/ethanol 4:1:1 mixture (ethanol can be replaced by THF). 73 mg of 10% palladium/carbon (10% Pd/C) are added, and then hydrogenation is carried out under atmospheric pressure for 15 h. The reaction mixture is filtered through prewashed kieselguhr, and the filtrate is concentrated in vacuo. The residue is mixed with 4.95 ml of 0.1N hydrochloric acid and concentrated. The residue is stirred with 10 ml of diethyl ether and decantered. The remaining solid is dried under high vacuum.

Yield: 103 mg (95% of theory).

HPLC (Method 1): Rₜ=3.04 min

LC-MS (Method 2): Rₜ=0.38 min

MS (EI): m/z=473 (M+H)⁺

¹H-NMR (400 MHz, D₂O): δ=2.06-220 (m, 1H), 2.74-2.89 (m, 1H), 2.94-3.05 (m, 1H), 3.12-3.25 (m, 2H), 3.53 (d, 1H), 3.61-3.72 (m, 1H), 3.97-4.07 (m, 1H), 4.53 (s, 1H), 4.61 (d, 1H), 4.76-4.91 (m, 12H), 7.01-7.05 (m, 2H), 7.07 (s, 1H), 7.40-7.45 (m, 2H), 7.51 (d, 1H).

### Example 21A

### Benzyl 2(S)-tert-butoxycarbonylamino-5-nitro-4-oxopentanoate

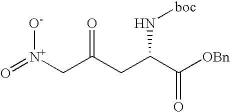

A solution A of 10 g (30.9 mmol) of 2(S)-tert-butoxycarbonylaminosuccinic acid 1-benzyl ester and 5.27 g (32.5 mmol) of 1,1′-carbonyldiimidazole in 100 ml of tetrahydrofuran is stirred at RT for 5 h. 18.8 g (30.9 mmol) of nitromethane are added dropwise to a solution B of 3.2 g (34.2 mmol) of potassium tert-butoxide in 100 ml of tetrahydrofuran at 0° C. Solution B is stirred while warming to RT, and then solution A is added dropwise at RT. The resulting mixture is stirred at RT for 16 h and adjusted to pH 2 with 20% strength hydrochloric acid. The solvent is evaporated. The remaining crude product is taken up in ethyl acetate/water. After separation of the phases, the organic phase is extracted twice with water, dried over sodium sulphate and concentrated. 13 g (99% of theory) of the product are obtained.

MS (ESI): m/z=334 (M+H)⁺

¹H-NMR (300 MHz, DMSO-d₆): δ=1.37 (s, 9H), 2.91 (m, 1H), 3.13 (m, 1H), 4.44 (m, 1H), 5.12 (s, 2H), 5.81 (m, 2H), 7.2-7.5 (m, 5H).

### Example 22A

### Benzyl 2(S)-tert-butoxycarbonylamino-4(R)-hydroxy-5-nitropentanoate

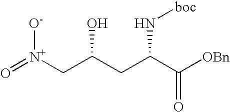

A solution of 11.3 g (30.8 mmol) of benzyl 2(S)-tert-butoxycarbonylamino-5-nitro-4-oxopentanoate in 300 ml of tetrahydrofuran is cooled to −78° C., 30.8 ml of a 1M solution of L-Selectrid® in tetrahydrofuran are added dropwise, and the mixture is stirred at −78° C. for 1 h. After warming to RT, saturated ammonium chloride solution is cautiously added to the solution. The reaction solution is concentrated, and the residue is taken up in water and ethyl acetate. The aqueous phase is extracted three times with ethyl acetate. The combined organic phases are dried over sodium sulphate and evaporated. The crude product is prepurified on silica gel 60 (mobile phase: cyclohexane/ethyl acetate 10/1), and the collected fractions are concentrated and stirred with cyclohexane/ethyl acetate 5/1. The remaining crystals are filtered off with suction and dried. 2.34 g (21% of theory) of the desired diastereomer are obtained. Chromatographic separation of the mother liquor on Lichrospher Diol 10 μm (mobile phase: ethanol/isohexane 5/95) results in a further 0.8 g (6.7% of theory) of the product.

MS (ESI): m/z=369 (M+H)⁺

¹H-NMR (300 MHz, DMSO-d₄): δ=1.38 (s, 9H), 1.77 (m, 1H), 1.97 (m, 1H), 4.10-4.44 (m, 3H), 4.67 (m, 1H), 5.12 (m, 2H), 5.49 (d, 1H), 7.25-7.45 (m, 5H).

### Example 23A

### Benzyl 2(S)-[S-benzyloxycarbonylamino-2(S)-tert-butoxycarbonylaminopentanoyl-amino]-3-{4,4′-bisbenzyloxy-3′-[2(S)-benzyloxycarbonylamino-2-(2-trimethyl-silylethoxycarbonyl)ethyl]biphenyl-3-yl}propionate

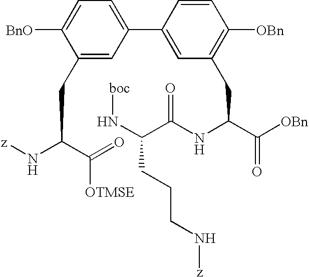

Preparation takes place in analogy to Example 16A from 0.47 g (0.51 mmol) of the compound from Example 15A and 0.19 g (0.51 mmol) of N⁵-[(benzyloxy)carbonyl]-N²-(tert-butoxycarbonyl)-L-ornithine with 0.19 g (0.51 mmol) of HATU and 0.35 ml (1.65 mmol) of N,N-diisopropylethylamine in 5.55 ml of dry DMF.

Yield: 0.58 g (92% of theory)

LC-MS (Method 10): Rₜ=3.46 min

MS (ESI): m/z=1212 (M+H)⁺

### Example 24A

### 2(S)-Benzyloxycarbonylamino-3-{4,4′-bisbenzyloxy-3′-[2(S)-benzyloxycarbonyl-2-(5-benzyloxycarbonylamino)-2(S)-tert-butoxycarbonylaminopentanoylamino)-ethyl]biphenyl-3-yl}-propionic acid

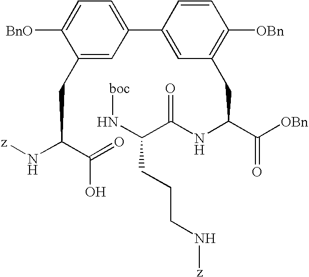

Preparation takes place in analogy to Example 17A from 0.82 g (0.68 mmol) of the compound from Example 23A with 2 equivalents (1.3 ml) of tetrabutylammonium fluoride (1M in THF) in 30 ml of dry DMF.

Yield: 772 mg (94% of theory)

LC-MS (Method 11): Rₜ=1.62 min

MS (ESI): m/z=1112 (M+H)⁺

### Example 25A

### Benzyl 2(S)-(5-benzyloxycarbonylamino-2(S)-tert-butoxycarbonylaminopentanoyl-amino)-3-[4,4′-bisbenzyloxy-3′-(2(S)-benzyloxycarbonylamino-2-pentafluoro-phenyloxycarbonylethyl)biphenyl-3-yl]propionate

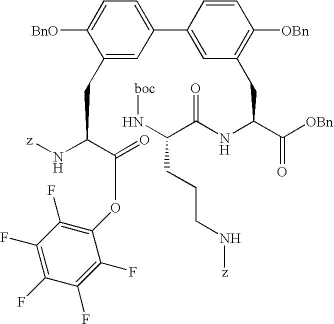

Preparation takes place in analogy to Example 18A from 422 mg (0.38 mmol) of the compound from Example 24A and 349 mg (1.9 mmol) of pentafluorophenol with 80 mg (0.42 mmol) of EDC and 4.63 mg (0.04 mmol) of DMAP in 4 ml of dichloromethane.

Yield: 502 mg (95% of theory)

LC-MS (Method 11): Rₜ=3.13 min

MS (ESI): m/z=1278 (M+H)⁺

### Example 26A

### Benzyl 2(S)-(5-benzyloxycarbonylamino-2(S)-aminopentanoylamino)-3-[4,4′-bis-benzyloxy-3′-(2(S)-benzyloxycarbonylamino-2-pentafluorophenyloxycarbonyl-ethyl)biphenyl-3-yl]propionate hydrochloride

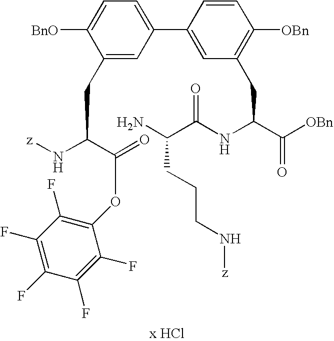

5 ml of a 4N solution of hydrogen chloride in dioxane are added to 215 mg (0.17 mmol) of the compound from Example 25A while stirring in an ice bath. The mixture is stirred for one hour and evaporated to constant weight in vacuo.

Yield: 200 mg (92% of theory)

LC-MS (Method 11): Rₜ=4.25 min

MS (ESI): m/z=1178 (M-HCl+H)⁺

### Example 27A

### Benzyl 5,17-bisbenzyloxy-14(S)-benzyloxycarbonylamino-11(s)-(3-benzyloxy-carbonylaminopropyl)-10,13-dioxo-9,12-diazatricyclo[14.3.1.1^{2,6}]henicosa-1(19),2,4,6(21),16(20),17-hexaene-8(S)-carboxylate

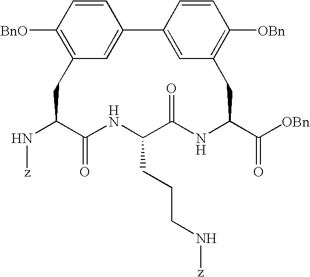

1.35 g (0.91 mmol) of the compound from Example 26A are introduced into 31 of chloroform and, while stirring vigorously, 2.54 ml (18.2 mmol) of triethylamine in 50 ml of chloroform are added at RT over the course of 20 min. The mixture is left to stir overnight and evaporated to dryness in vacuo. The residue is stirred with 5 ml of acetonitrile and filtered, and the residue is dried to constant weight.

Yield: 890 mg (93% of theory)

LC-MS (Method 11): Rₜ=5.10 min

MS (ESI): m/z=994 (M+H)⁺

### Example 28A

### (8S,11S,14S)-14-Amino-11-(3-aminopropyl)-5,17-dihydroxy-10,13-dioxo-9,12-diazatricyclo[14.3.1.1^{2,6})henicosa-1(20),2(21),3,5,6,18-hexaene-8-carboxylic acid dihydrochloride

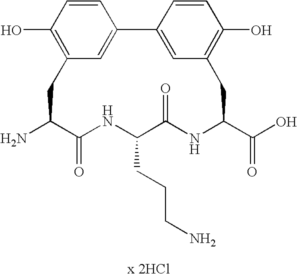

50 mg (0.05 mmol) of the compound from Example 27A are suspended in 50 ml of glacial acetic acid/water/ethanol (4/1/1), 30 mg of Pd/C (10%) catalyst are added, and the mixture is hydrogenated at RT for 20 hours. After removal of the catalyst by filtration through kieselguur, the filtrate is evaporated to dryness in vacuo and, while stirring, 2.5 ml of 0.1N hydrochloric acid are added. The mixture is evaporated to dryness in vacuo and dried to constant weight.

Yield: 17 mg (63% of theory)

TLC (methanol/dichloromethane/25% ammonia=5/3/2): R_{f}=0.6

LC-MS (Method 3): Rₜ=0.28 min

MS (ESI): m/z=457 (M-2HCl+H)⁺

### Example 29A

### (8S,11S,14S)-14-[(tert-Butoxycarbonyl)amino-11-[3-[(tert-butoxycarbonyl)-amino]propyl}-5,17-dihydroxy-10,13-dioxo-9,12-diazatricyclo[14.3.1.1^{2,6}]henicosa-1(20),2(21),3,5,16,18-hexaene-8-carbonic acid

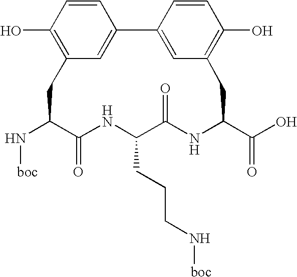

600 mg (1.13 mmol) of the compound from Example 28A are dissolved in 6 ml (5.66mmol) of 1N sodium hydroxide solution and, while stirring at room temperature, 740.8 mg (3.39 mmol) of di-tert-butyl dicarbonate, dissolved in 5 ml of methanol, are added. The reaction is complete after one hour (TLC check, mobile phase: dichloromethane/methanol/ammonia=80/20/2). The pH is adjusted to 3 by dropwise addition of 0.1N hydrochloric acid. Extraction three times with 20 ml of ethyl acetate each time and drying with sodium sulphate are followed by evaporation to constant weight in vacuo.

Yield: 622 mg (84% of theory)

LC-MS (Method 10): Rₜ=1.96 min

MS (ESI): m/z=656 (M+H)⁺

### Example 30A

### 2-(Benzyloxy)-N-(tert-butoxycarbonyl)-5-iodo-N-methyl-L-phenylalanine

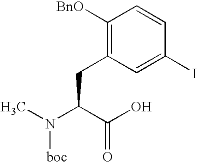

Under an argon atmosphere, 500 mg (1 mmol) of the compound from Example (−)-6A are dissolved in 20 ml of THF, 90.5 mg (3.02 mmol) of sodium hydride and 0.51 ml (1141.6 mg; 8.04 mmol) of methyl iodide (80% pure) are added, and the mixture is stirred at room temperature overnight. It is diluted with 25 ml of ethyl acetate and 25 ml of water and adjusted to pH=9 with 0.1N hydrochloric acid. The mixture is concentrated to a small volume in vacuo. 10 ml of ethyl acetate and 10 ml of water are added, the mixture is shaken vigorously, and the organic phase is separated off. Drying with sodium sulphate and concentration in vacuo result in 140 mg of product (19% of theory). The aqueous phase is acidified (pH=3) and extracted three times with 20 ml of ethyl acetate. Concentration in vacuo and drying in vacuo result in 351 mg of product (68% of theory).

LC-MS (Method 9): Rₜ=3.9 min

MS (EI): m/z=511 (M+H)⁺

### Example 31A

### Benzyl 2-(benzyloxy)-N-(tert-butoxycarbonyl)-5-iodo-N-methyl-L-phenylalaninate

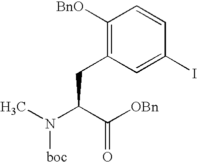

Preparation takes place in analogy to Example 7A from 350 mg (0.68 mmol) of the compound from Example 30A, 8.29 mg (0.07 mmol) of DMAP, 148 mg (1.37 mmol) of benzyl alcohol and 157.46 mg (0.82 mmol) of EDC in 3 ml of acetonitrile.

Yield: 382 mg (93% of theory)

LC-MS (Method 9): Rₜ=4.8 min

MS (EI): mi/z=601 (M+H)⁺

### Example 32A

### Benzyl 2-(benzyloxy)-N-(tert-butoxycarbonyl)-N-methyl-5-(4,4,5,5-tetramethyl-1,3,2-dioxaborolan-2-yl)-L-phenylalaninate

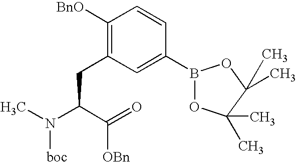

In analogy to Example 8A, 380 mg (0.63 mmol) of the compound from Example 31A are introduced into 4 ml of DMF in a heat-dried flask and, while stirring at room temperature, 184.5 mg (0.73 mmol) of 4,4,4′,4′,5,5,5′,5′-octamethyl-2,2′-bi-1,3,2-dioxaborolane, 186 mg (1.9 mmol) of potassium acetate and 23.15 mg (0.03 mmol) of bis(diphenylphosphino)ferrocenepalladium(II) chloride are added. Reaction is allowed to take place at 80° C. for 4 h. The product is obtained after workup and chromatography (silica gel 60, mobile phase: cyclohexane/ethyl acetate=4/1).

Yield: 196 mg

LC-MS (Method 9): Rₜ=4.9 min

MS (EI): m/z=601 (M+H)⁺

### Example 33A

### 2-(Trimethylsilyl)ethyl 2(S)-benzyloxycarbonylamino-3-[4,4′-bisbenzyloxy-3′-(2(S)-benzyloxycarbonyl-(2-tert-butoxycarbonyl-2-methyl)aminoethyl)biphenyl-3-yl]propionate

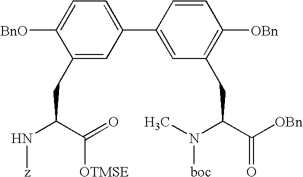

Preparation takes place in analogy to Example 12A from 190 mg (0.32 mmol) of the compound from Example 32A, 199.5 mg (0.32 mmol) of the compound from Example 11A, 195.5 mg (0.63 mmol) of caesium carbonate and 23.15 mg (0.03 mmol) of bis(diphenylphosphino)ferrocenepalladium(II) chloride in 1.5 ml of DMF under an argon atmosphere.

Yield: 212 mg (66% of theory)

LC-MS (Method 13): Rₜ=4.86 min

MS (EI): ml/z=978 (M+H)⁺

### Example 34A

### 2-(Trimethylsilyl)ethyl 2(S)-benzyloxycarbonylamino-3-[4,4′-bisbenzyloxy-3′-(2(S)-benzyloxycarbonyl-2-methylaminoethylbiphenyl-3-yl]propionate hydrochloride

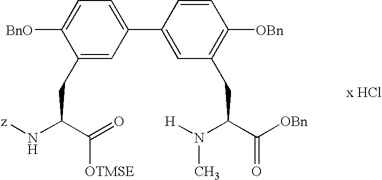

Preparation takes place in analogy to Example 15A from 930 mg (0.95 mmol) of the compound from Example 33A and 22.14 ml of a 4M solution of hydrogen chloride in dioxane, in 15 ml of dioxane.

Yield: 915 mg (78% of theory)

LC-MS (Method 13): Rₜ=2.53 min

MS (EI): m/z=878 (M-HCl+H)⁺

### Example 35A

### Benzyl 2(S)-{Methyl-[5-benzyloxycarbonylamino-2(S)-tert-butoxycarbonylamino-4(R)-(tert-butyldimethylsilyloxy)pentanoyl]amino}-3-{4,4′-bisbenzyloxy-3′-[2(S)-benzyloxycarbonylamino-2-(2-trimethylsilylethoxycarbonyl)ethyl]biphenyl-3-yl)propionate

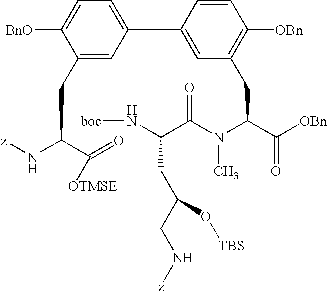

Preparation takes place in analogy to Example 16A from 922 mg (1.01 mmol) of the compound from Example 34A, 0.5 g (1.01 mmol) of the compound from Example 14A, 421 mg (1.11 mmol) of HATU and 0.7 ml (518 mg; 3.27 mmol) of DIEA in 4.2 ml of DMF.

Yield: 703 mg (51% of theory)

LC-MS (Method 8): Rₜ=3.17 min

MS (EI): m/z=1356 (M+H)⁺

### Example 36A

### 2(S)-Benzyloxycarbonylamino-3-{4,4′-bisbenzyloxy-3′-[2(S)-benzyloxycarbonyl-2-{methyl-(5-benzyloxycarbonylamino-2(S)-tert-butoxycarbonylamino-4(R)-hydroxypentanoyl)amino}ethyl]biphenyl-3-yl}propionic acid

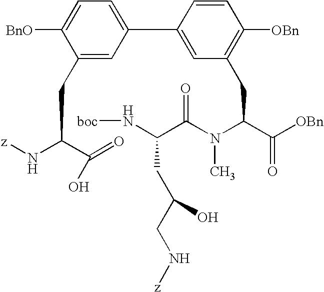

Preparation takes place in analogy to Example 17A from 360 mg (0.27 mmol) of the compound from Example 35A and 0.8 ml (3 equivalents) of 1M tetrabutylammonium fluoride solution (THF) in 20 ml of DMF.

Yield: 159 mg (53% of theory)

LC-MS (Method 12): Rₜ=3.19 min

MS (EI): m/z=1142 (M+H)⁺

### Example 37A

### Benzyl 2(S)-[methyl-(5-benzyloxycarbonylamino)-2(S)-tert-butoxycarbonylamino-4(R)-hydroxypentanoyl]amino-3-[4,4′-bisbenzyloxy-3′-(2(S)-benzyloxy-carbonylamino-2-pentafluorophenyloxycarbonylethyl)biphenyl-3-yl]propionate

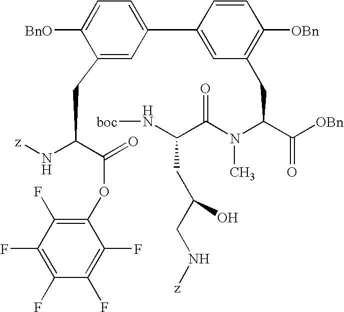

Preparation takes place in analogy to Example 18A from 330 mg (0.29 mmol) of the compound from Example 36A, 265.6 mg (1.44 mmol) of pentafluorophenol, 3.53 mg (0.03 mmol) of DMAP and 60.87 mg (0.32 mmol) of EDC in 10 ml of dichloromethane.

Yield: 271 mg (69% of theory)

LC-MS (Method 12): Rₜ=3.38 min

MS (EI): m/z=1308 (M+H)⁺

### Example 38A

### Benzyl 2(S)-[methyl-(5-benzyloxycarbonylamino)-2(S)-amino-4(R)-hydroxy-pentanoyl]amino-3-[4,4′-bisbenzyloxy-3′-(2(S)-benzyloxycarbonylamino-2-pentafluorophenyloxycarbonylethyl)biphenyl-3-yl]propionate hydrochloride

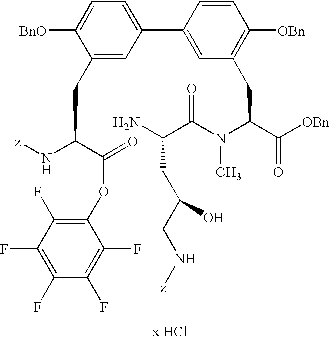

130 mg (0.1 mmol) of the compound from Example 37A are dissolved in 0.5 ml of dioxane, and 5 ml of a 4N solution of hydrogen chloride in dioxane are cautiously added (ice bath). After 30 minutes, reaction is allowed to continue at room temperature for a further 2 h. The mixture is evaporated to dryness in vacuo and dried to constant weight under high vacuum.

Yield: 130 mg (70% of theory)

LC-MS (Method 15): Rₜ=2.68 min

MS (EI): m/z=1208 (M-HCl+H)⁺

### Example 39A

### Benzyl (8S,11S,14S)-5,17-bis(benzyloxy)-14-{[(benzyloxy)carbonyl]amino}-11-((2R)-3-{[(benzyloxy)carbonyl]amino}-2-hydroxypropyl-9-methyl-10,13-dioxo-9,12-diazatricyclo[14:3.1.1^{2.6}]henicosa-1(20),2(21),3,5,16,18-hexaene-8-carboxylate

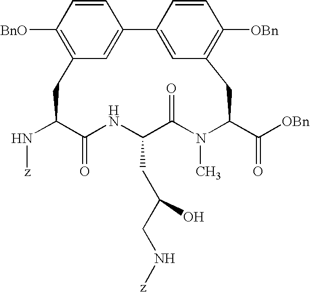

130 mg (0.1 mmol) of the compound from Example 38A are introduced into 220 ml of dry chloroform. While stirring at room temperature, 23 ml (20 eq.) of triethylamine in 5 ml of dichloromethane are added over the course of 20 minutes. The mixture is stirred overnight. It is then evaporated to dryness in vacuo. The residue is stirred with acetonitrile. Drying of the residue results in 44 mg of product Further product (30 mg) is obtained from the mother liquor by RP-HPLC.

Yield: 74 mg (69% of theory)

LC-MS (Method 15): Rₜ=3.13 min

MS (EI): m/z=1024 (M+H)⁺

### Example 40A

### (8S,11S,14S)-14-Amino-11-[(2R)-3-amino-2-hydroxypropyl]-5,17-dihydroxy-9-methyl-10,13-dioxo-9,12-diazatricyclo[14.3.1.1^{2,6}]henicosa-1(20),2(21),3,5,16,18-hexaene-8-carboxylic acid di(trifluoroacetate)

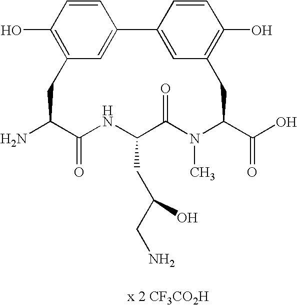

33 mg (0.032 mmol) of the compound from Example 39A are cautiously treated with dilute trifluoro acetic acid. The resulting clear solution is subsequently lyophilized.

Yield: 23 mg (quantitative)

LC-MS (Method 15): Rₜ=0.92 min

MS (EI): m/z=486 (M-2CF₃CO₂H+H)⁺

### Example 41A

### (8S,11S,14S)-5,17-Bis(benzyloxy)-14-{[benzyloxycarbonyl]amino}-11-(2R)-3-{[benzyloxycarbonyl]amino}-2-hydroxypropyl-9-methyl-10,13-dioxo-9,12-diazatricyclo[14.3.1.1^{2,6}]henicosa-1(20),2(21),3,5,16,18-hexaene-8-carboxylic acid

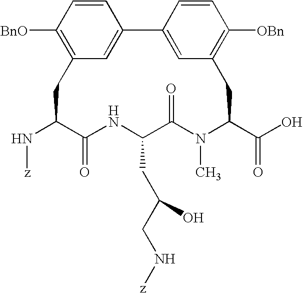

37 mg (0.04 mmol) of the compound from Example 39A are dissolved in 2 ml of THF, 0.14 ml of 1N lithium hydroxide solution is added, and the mixture is stirred at room temperature for 3 h. It is then acidified with 1N hydrochloric acid and evaporated to dryness under high vacuum.

Yield: 33 mg (71% of theory)

LC-MS (Method 12): Rₜ=2.90 min

MS (EI): m/z=934 (M+H)⁺

Examples 42A to 48A listed in the following table are prepared from the appropriate starting compounds in analogy to the methods detailed above:

| Ex. | | Prepared in | |
|---|---|---|---|
| No. | Structure | analogy to | Analytical data |
| 42A | 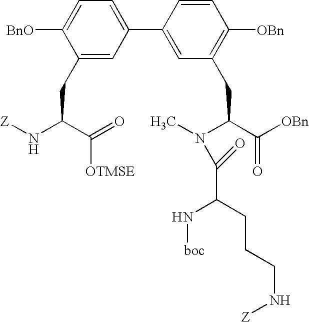 | 16A with N⁵- [(benzyloxy)- carbonyl]-N²- (tert-butoxy- carbonyl)-L- ornithine | LC-MS (Method 13): Rₜ = 4.85 min. MS (EI): m/z = 1226 (M + H)⁺ |
| 43A | 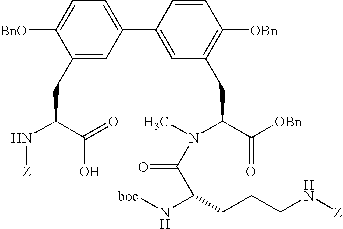 | 17A | LC-MS (Method 13): Rₜ = 2.04 min. MS (EI): m/z = 1126 (M + H)⁺ |
| 44A | 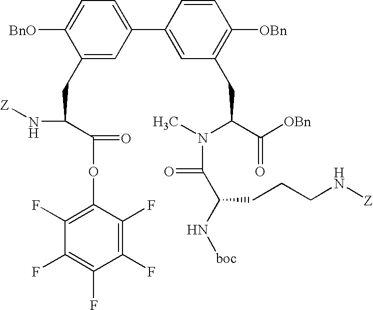 | 18A | LC-MS (Method 13): Rₜ = 3.79 min. MS (EI): m/z = 1292 (M + H)⁺ |
| 45A | 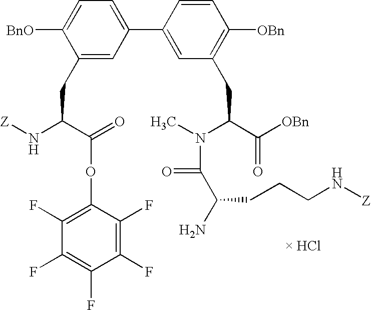 | 26A | LC-MS (Method 13): Rₜ = 3.72 min. MS (EI): m/z = 1192 (M-HCl + H)⁺ |
| 46A | 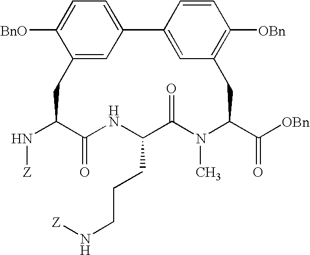 | 27A | LC-MS (Method 13): Rₜ = 4.39 min. MS (EI): m/z = 1008 (M + H)⁺ |
| 47A | 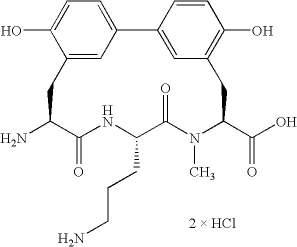 | 28A | LC-MS (Method 12): Rₜ = 0.53 min. MS (EI): m/z = 470 (M-2HCl + H)⁺ |
| 48A | 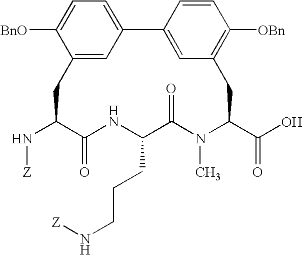 | 41A | LC-MS (Method 14): Rₜ = 3.64 min. MS (EI): m/z = 918 (M + H)⁺ |

### Example 49A

### 2-(Trimethylsilyl)ethyl(2Z)-3-[2-(benzyloxy)-5-bromophenyl]-2-{[(benzyloxy)-carbonyl]amino}acrylate

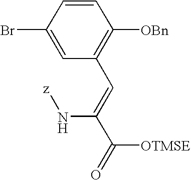

7.5 g (25.8 mmol) of 2-(benzyloxy)-5-bromobenzaldehyde (Synthesis, 1992, 10, 1025-30) and 11.8 g (28.3 mmol) of 2-(trimethylsilyl)ethyl{[(benzyloxy)carbonyl]amino}(dimethoxyphosphoryl)acetate (Tetrahedron, 1999, 55, 10527-36) are introduced in 150 ml of THF and, while cooling at −78° C. in acetone/dry ice, 3.26 g (28.3 mmol) of 1,1,3,3-tetramethylguanidine are added. The mixture is slowly warmed to RT and stirred at RT for a further 12 h. The solvent is distilled off in vacuo, and the crude product is taken up in ethyl acetate and washed once each with saturated sodium bicarbonate solution and saturated sodium chloride solution. The organic phase is dried over magnesium sulphate, filtered and concentrated to dryness in vacuo. The crude product is recrystallized from ethyl acetate/cyclohexane (1:20).

Yield: 13 g (88% of theory)

HPLC (Method 16): Rₜ=6.06 min

MS (DCI(NH₃)): m/z=599 (M+NH₄)⁺

### Example 50A

### (8S,11S,14S)-5,17-Bis(benzyloxy)-14-{[(benzyloxy)carbonyl]amino}-11-(3-{[(benzyloxy)carbonyl]amino}propyl)-10,13-dioxo-9,12-diazatricyclo[14.3.1.1^{2,6}]-henicosa-1(20),2(21),3,5,16,18-hexaene-8-carboxylic acid

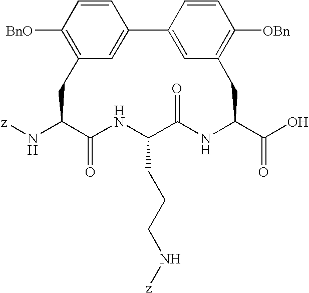

200 mg (0.2 mmol) of the compound from Example 27A are introduced into 8 ml of THF and 4 ml of DMF and, while stirring, 0.8 ml of a 1M aqueous lithium hydroxide solution (4 equivalents) is added. A gel is produced after stirring at room temperature for 2 h. 0.8 ml of 1N hydrochloric acid and a little water are added. The mixture is then evaporated to dryness in vacuo, stirred with water, and the precipitate is filtered and dried.

Yield: 140 mg (77% of theory)

LC-MS (Method 10): Rₜ=2.83 min

MS (EI): m/z=904 (M+H)⁺

### Example 51A

### (8S,11S,14S)-14-[(tert-Butoxycarbonyl)amino]-11-{3-[(tert-butoxycarbonyl)amino]-propyl}-5,17-dihydroxy-9-methyl-10,13-dioxo-9,12-diazatricyclo[14.3.1.1^{2,6}]-henicosa-1(20),2(21),3,5,16,18-hexaene-8-carboxylic acid

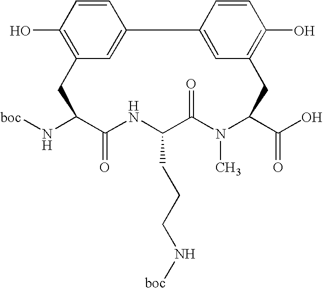

11 mg (0.02 mmol) of the compound from Example 47A are dissolved in 0.5 ml of water, 12.27 mg (0.08 mmol) of sodium carbonate are added, the mixture is cooled in an ice bath and, while stirring, 13.25 mg (0.06 mmol) of di-tert-butyl dicarbonate in 0.2 ml of methanol are added. The mixture is stirred at RT overnight, evaporated to dryness in vacuo, dissolved in 0.5 ml of water and acidified to pH=2 with 1N hydrochloric acid, and the resulting suspension is extracted with ethyl acetate. Drying with sodium sulphate is followed by evaporation to dryness in vacuo.

Yield: 10 mg (51% of theory)

LC-MS (Method 12): Rₜ=1.92 min

MS (EI): m/z=670 (M+H)⁺

### Example 52A

### (8S,11S,14S)-14-[(tert-Butoxycarbonyl)amino]-11-{(2R)-3-[(tert-butoxycarbonyl)-amino]-2-hydroxypropyl}-5,17-dihydroxy-9-methyl-10,13-dioxo-9,12-diazatricyclo[14.3.1.1^{2,6}]henicosa-1(20),2(21),3,5,16,18-hexaene-8-carboxylic acid

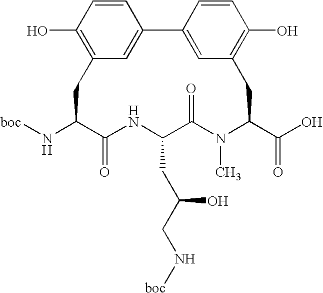

90 mg (0.16 mmol) of the compound from Example 40A are dissolved in 2.5 ml of water, 85.3 mg (0.8 mmol) of sodium carbonate are added, the mixture is cooled in an ice bath, and 105.3 mg (0.48 mmol) of di-(tert-butyl) dicarbonate in 1.2 ml of methanol are added. The mixture is stirred at room temperature overnight, concentrated in vacuo to a small volume and acidified to pH=2 with 1N hydrochloric acid. The resulting precipitate is filtered off and dried.

Yield: 89 mg (73% of theory)

LC-MS (Method 12): Rₜ=1.8 min

MS (EI): m/z=686 (M+H)⁺

### Example 53A

### 2-(Trimethylsilyl)ethyl 2-(benzyloxy)-N-[(benzyloxy)carbonyl]-5-bromo-L-phenylalaninate

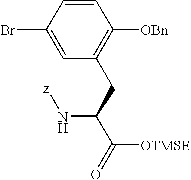

930 mg (1.6 mmol) of the compound from Example 49A are dissolved in 100 ml of ethanol and 10 ml of dioxane. Under an argon atmosphere, 20 mg of (+)-1,2-bis((2S,5S)-2,5-diethylphospholano)benzene(cyclooctadiene)rhodium(I) trifluoromethanesulphonate are added, and the solution is left in an ultrasonic bath for 15 min. Hydrogenation is then carried out under a hydrogen pressure of 3 bar for 5 d. The mixture is filtered through silica gel and carefully washed with ethanol. The filtrate is concentrated in vacuo, and the crude product is dried under high vacuum.

Yield: 900 mg (96% of theory)

ee=98.8% (determined by analytical HPLC: Chiralcel OD (Daicel); eluent: i-hexane and ethanol (5/1 vol/vol) with addition of 0.2% by volume diethylamine)

HPLC (Method 16): Rₜ=6.08 min

MS (DCI(NH₃)): m/z=601 (M+NH₄)⁺

### Example 54A

### Methyl(2Z)-3-[2-(benzyloxy)-5-bromophenyl]-2-{[(benzyloxy)carbonyl]amino}-acrylate

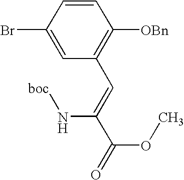

Preparation takes place in analogy to Example 49A from 7.5 g (25.8 mmol) of 2-(benzyloxy)-5-bromobenzaldehyde and 8.4 g (28.3 mmol) of 2-(trimethylsilyl)ethyl{[benzyloxy)carbonyl]amino}(dimethoxyphosphoryl)acetate (J. Prakt. Chem., 2000, 342, 736-44) with 3.3 g (28.3 mmol) of 1,1,3,3-tetramethylguanidine in 150 ml of THF.

Yield: 10 g (87% of theory)

HPLC (Method 16): Rₜ=5.42 min

MS (DCI(NH₃)): m/z=479 (M+NH₄)⁺

### Example 55A

### Methyl 2-(benzyloxy)-N-[(benzyloxy)carbonyl]-5-bromo-L-phenylalaninate

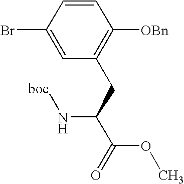

Preparation takes place in analogy to Example 53A from 1.96 g (4.2 mmol) of the compound from Example 54A and 15 mg of (+)-1,2-bis((2S,5S)-2,5-diethylphospholano)benzene(cyclooctadiene)rhodium(I) trifluoromethanesulphonate in 100 ml of ethanol and 20 ml of dioxane.

Yield: 1.96 g (99% of theory)

ee=97.6% (determined by analytical HPLC: Chiralcel OD (Daicel); eluent: i-hexane and ethanol (5/1 vol/vol) with addition of 0.2% by volume diethylamine)

LC-MS (Method 17): Rₜ=3.05 min

MS (DCI(NH₃)): m/z=481 (M+NH₄)⁺

¹H-NMR (300 MHz, DMSO-d₆): δ=1.32 (s, 9H), 2.72 (m_{c}, 1H), 3.17 (m_{c}, 1H), 3.60 (s, 3H), 4.32 (m_{c}, 1H), 5.13 (s, 2H), 7.01 (m_{c}, 1H), 7.22 (m_{c}, 1 H), 7.28-7.58 (m_{c}, 6H).

### Example 56A

### Methyl 2-(benzyloxy)-N-(tert-butoxycarbonyl)-5-(4,4,5,5-tetramethyl-1,3,2-dioxaborolan-2-yl)-L-phenylalaninate

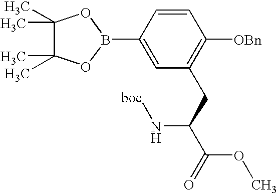

0.23 g (2.31 mmol) of potassium acetate and 4 mg (0.08 mmol) of potassium hydroxide are added to a solution of 0.36 g (0.77 mmol) of the compound from Example 55A in 5 ml of DMF. The mixture is deoxygenated by passing argon through the vigorously stirred solution for 15 min. Then 0.25 g (1.0 mmol) of 4,4,4′,4′,5,5,5′,5′-octamethyl-2,2′-bi-1,3,2 dioxaborolane and 0.023 g (0.03 mmol, 0.04 equivalent) of bis(diphenylphosphino)ferrocenepalladium(II) chloride are added. The mixture is heated under a gentle stream of argon to 60° C. and stirred at this temperature for 1.5 h. It is subsequently stirred at 80° C. for 30 min and then cooled to RT. The solvent is distilled off in vacuo, and the crude product is taken up in ethyl acetate and washed twice with saturated sodium chloride solution. The organic phase is dried over magnesium sulphate, filtered and evaporated to dryness in vacuo. The residue is purified by chromatography (RP-HPLC, acetonitrile, water).

Yield: 0.219 g (56% of theory)

MS (EI): m/z=512 (M+H)⁺

¹H-NMR (400 MHz, DMSO-d₆): δ=1.27 (m_{c}, 12H), 1.29 (s, 9H), 2.75 (m_{c}, 1H), 3.19 (m_{c}, 1H), 3.57 (s, 3H), 4.30 (m_{c}, 1H), 5.19 (m_{c}, 2H), 7.04 (m_{c}, 1H), 7.24 (m_{c}, 1 H), 7.28-7.58 (m, 6H).

### Example 57A

### 2-(Trimethylsilyl)ethyl 2-(benzyloxy)-N-[(benzyloxy)carbonyl]-5-(4,4,5,5-tetramethyl-1,3,2-dioxaborolan-2-yl)-L-phenylalaninate

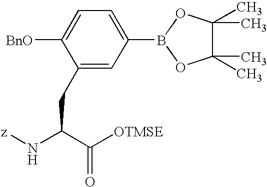

Preparation takes place in analogy to Example 8A from 2.0 g (3.17 mmol) of the compound from Example 11A, 0.924 g (3.64 mmol) of 4,4,4′,4′,5,5,5′,5′-octamethyl-2,2′-bi-1,3,2-dioxaborolane, 0.932 g (9.50 mmol) of potassium acetate and 0.116 g (0.160 mmol, 0.05 equivalent) of bis(diphenylphosphino)-ferrocenepalladium(II) chloride in 30 ml of dimethyl sulphoxide.

Yield: 1.12 g (56% of theory)

LC-MS (Method 13): Rₜ=4.50 min

MS (EI): m/z=632 (M+H)⁺

¹H-NMR (200 MHz, CDCl₃): δ=0.92 (dd, 2H), 1.31 (s, 12H), 2.95-3.95 (m, 2H), 4.11 (m_{c}, 2H), 4.55 (11 (m_{c}, 1H), 4.99 (s, 2H), 5.08 (s, 2H), 5.53 (d, 1H), 6.90 (d, 1H), 7.15-7.47 (m, 10 H), 7.58 (d, 1H), 7.67 (dd, 1H).

### Example 58A

### 2-(Trimethylsilyl)ethyl(2S)-2-{[(benzyloxyl)carbonyl]amino)-3-(4,4′-bis(benzyl-oxy)-3′-{(2S)-2-[(tert-butoxycarbonyl)amino]-3-methoxy-3-oxopropyl}biphenyl-3-yl)propanoate

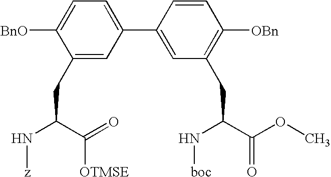 Method A:

Preparation takes place in analogy to Example 12A from 0.46 g (0.79 mmol) of the compound from Example 53A, 0.41 g (0.79 mmol) of the compound from Example 56A, 0.52 g (1.58 mmol) of caesium carbonate and 0.023 g (0.032 mmol, 0.04 equivalent) of bis(diphenylphosphino)ferrocenepalladium(II) chloride in 12 ml of DMF.

Yield: 0.34 g (48% of theory)

Method B:

Preparation takes place in analogy to Example 53A from 0.59 g (0.67 mmol) of the compound from Example 78A and 10 mg of (+)-1,2-bis((2S,5S)-2,5-diethylphospholano)benzene(cyclooctadiene)rhodium(I) trifluoromethanesulphonate in 100 ml of ethanol and 30 ml of dioxane.

Yield: 0.60 g (99% of theory)

ee=99.5% (determined by analytical HPLC: chiral silica gel selector packing material KBD 8361 (250 mm×4.6 mm) based on the selector poly(N-methacryloyl-L-leucine 1-menthylamide); temperature: 23° C.; flow rate: 1 m/min; eluent: i-hexane and ethyl acetate (2/1 vol/vol))

HPLC (Method 16): Rₜ=6.54 min

MS (EI): m/z=890 (M+H)⁺¹H-NMR (400 MHz, DMSO₆): δ=0.00 (s, 9H), 0.83 (m_{c}, 2H), 1.31 (s, 9H), 2.86 (m, 2H), 3.25 (m, 2H), 3.62 (s, 3H), 4.09 (m, 2H), 4.41 (m_{c}, 1H), 4.98 (m_{c}, 2H), 5.22 (m, 4H), 7.12 (m, 2H), 7.29 (m, 2H), 7.33-7.59 (m, 20 H), 7.78 (d, 1H).

Examples 59A to 64A listed in the following table are prepared in analogy to the methods detailed above from the appropriate starting compounds:

| | | Preparation | |
|---|---|---|---|
| Example | | in analogy | |
| No. | Structure | to | Analytical data |
| 59A | 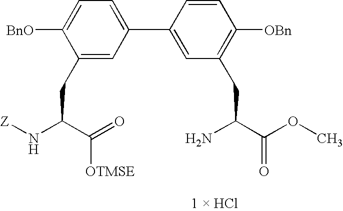 | 15A | LC-MS (Method 12): Rₜ = 2.50 min. MS (EI): m/z = 789 (M-HCl + H)⁺ |
| 60A | 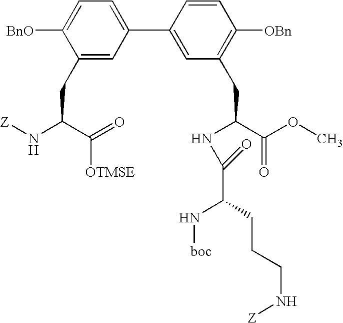 | 16A | LC-MS (Method 13): Rₜ = 3.51 min. MS (EI): m/z = 1137 (M + H)⁺ |
| 61A | 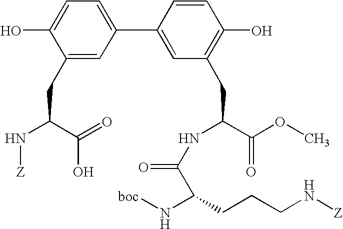 | 17A | LC-MS (Method 13): Rₜ = 3.20 min. MS (EI): m/z = 1037 (M + H)⁺ |
| 62A | 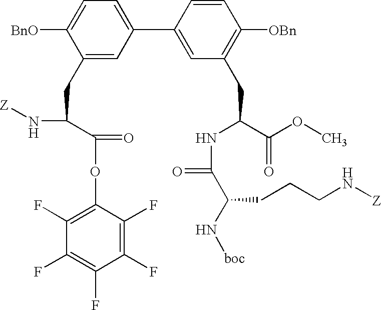 | 18A | LC-MS (Method 19): Rₜ = 3.43 min. MS (EI): m/z = 1203 (M + H)⁺ |
| 63A | 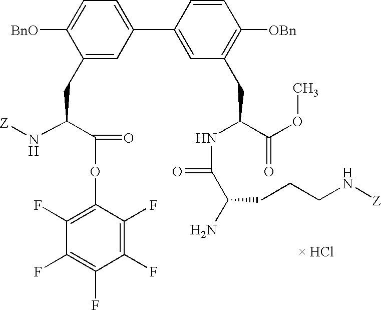 | 26A | LC-MS (Method 12): Rₜ = 2.83 min. MS (EI): m/z = 1103 (M-HCl + H)⁺ |
| 64A | 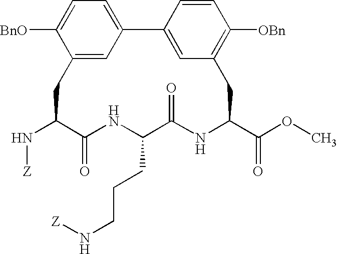 | 39A | LC-MS (Method 12): Rₜ = 3.10 min. MS (EI): m/z = 919 (M + H)⁺ |

### Example 65A

### (8S,11S,14S)-14-[(tert-Butoxycarbonyl)amino]-11-{(2R)-3-[(tert-butoxycarbonyl)amino]-2-hydroxypropyl}-5,17-dihydroxy-10,13-dioxo-9,12-diazatricyclo[14.3.1.1^{2,6}]henicosa-1(20),2(21),3,5,16,18-hexaene-8-carboxylic acid

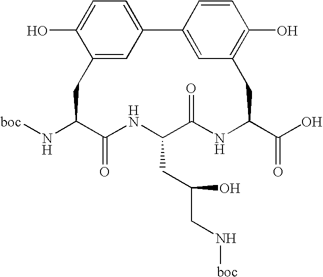

50 mg (0.09 mmol) of the compound from Example 20A are introduced into 8 ml of a methanol/water (9:1) mixture. 1 ml of 1N sodium bicarbonate solution and then 80 mg (0.37 mmol) of di-tert-butyl dicarbonate in 2 ml of methanol/water (9:1) are added. The mixture is stirred at RT overnight. The solution is worked up by adding 60 ml of ethyl acetate and 30 ml of water. The organic phase is washed once with 0.1N hydrochloric acid, dried and concentrated in vacuo.

Yield: 49 mg (79% of theory)

LC-MS (Method 3): Rₜ=2.56 min

MS (EI): m/z=673 (M+H)⁺

LC-HR-FT-ICR-MS: calc. for C₃₃H₄₄N₄O₁₁ (M+H)⁺673.3079 found 673.3082.

### Example 66A

### Benzyl (8S,11S,14S)-5,17-bis(benzyloxy)-14-{[(benzyloxy)carbonyl]amino}-11-((2R)-3-{[(benzyloxy)carbonyl]amino}-2-{[tert-butyl(dimethyl)silyl]oxy}propyl)-10,13-dioxo-9,12-diazatricyclo[14.3.1.1^{2,6}]henicosa-1(20),2(21),3,5,16,18-hexaene-8-carboxylate

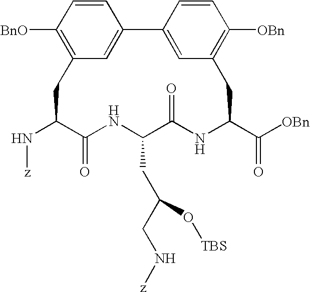

200 mg (0.20 mmol) of the compound from Example 19A are dissolved in 50 ml of absolute DMF and, at 0° C., 210 mg (0.79 mmol) of tert-butyldimethylsilyl trifluoromethanesulphonate, 0.11 ml (0.79 mmol) of triethylamine and 20 mg (0.20 mmol) of DMAP are added. The mixture is stirred at RT for 2 d. After addition of 20 ml of methylene chloride, the solution is cautiously washed with 10 ml of saturated sodium bicarbonate solution and 10 ml of water. The organic phase is concentrated to dryness, and the residue is dried under high vacuum.

Yield: 215 mg (96% of theory)

LC-MS (Method 12): Rₜ=3.43 min

MS (EI): m/z=1125 (M+H)⁺

### Example 67A

### (8S,11S,14S)-5,17-Bisbenzyloxy)-14-{[(benzyloxy)carbonyl]amino}-11-((2R)-3-{[(benzyloxy)carbonyl]amino}-2-{[tert-butyl(dimethyl)silyl]oxy}propyl)-10,13-dioxo-9,12-diazatricyclo[14.3.1.1^{2,6}]henicosa-1(20),2(21),3,5,16,18-hexaene-8-carboxylic acid

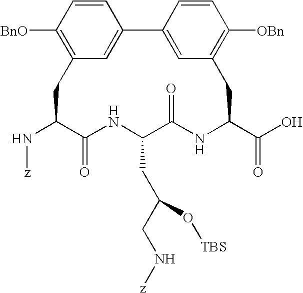

210 mg (0.19 mmol) of the compound from Example 66A are dissolved in 2 ml of THF, and 1 ml each of water and methanol are added. Addition of 13 mg (0.56 mmol) of lithium hydroxide is followed by stirring at RT for 12 h. The reaction solution is then diluted with 30 ml of water and adjusted to pH=3 by adding 1N hydrochloric acid. The precipitate is filtered off and dried under high vacuum.

Yield: 192 mg (99% of theory)

LC-MS (Method 12): Rₜ=3.24 min

MS (EI): m/z=1135 (M+H)⁺

### Example 68A

### tert-Butyl{(2R)-3-[(8S,11S,14S)-14-[(tert-butoxycarbonyl)amino]-8-[({2-[(tert-butoxycarbonyl)amino]ethyl}amino)carbonyl]-5,17-dihydroxy-9-methyl-10,13-dioxo-9,12-diazatricyclo[14.3.1.1^{2,6}]henicosa-1(20),2(21),3,5,16,18-hexaen-11-yl]-2-hydroxypropyl}carbamate

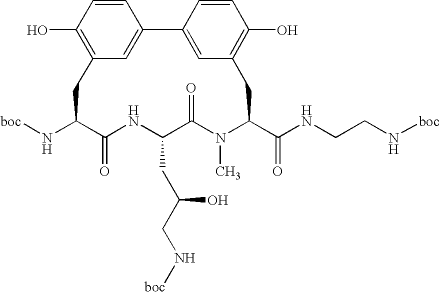

65 mg (0.09 mmol) of the compound from Example 52A and 18.2 mg (0.11 mmol) of tert-butyl (2-aminoethyl)carbamate are dissolved in 2 ml of abs. DMF and cooled in an ice bath, and 43.19 mg (0.11 mmol) of HATU and 16.31 mg (0.13 mmol) of Hünig's base are added. The mixture is then stirred at RT for 30 min, a further 36.62 mg (0.26 mmol) of Hünig's base are added, and the reaction is allowed to continue with stirring overnight. The mixture is evaporated to dryness in vacuo and the residue is chromatographed by RP-HPLC (acetonitrile, water).

Yield: 42 mg (54% of theory)

LC-MS (Method 17): Rₜ=2.31 min

MS (EI): m/z=828 (M+H)⁺

### Example69A

### tert-Butyl 4-({[(8S,11S,14S)-14-[(tert-butoxycarbonyl)amino]-11-{(2R)-3-[(tert-butoxycarbonyl)amino]-2-hydroxypropyl}-5,17-dihydroxy-9-methyl-10,13-dioxo-9,12-diazatricyclo[14.3.1.1^{2,6}]henicosa-1(20),2(21),3,5,16,18-hexaen-8-yl]carbonyl}amino)piperidine-1-carboxylate

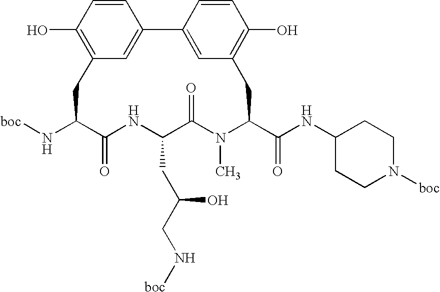

Preparation takes place in analogy to Example 68A from 20 mg (0.03 mmol) of the compound from Example 52A and 7 mg (0.03 mmol) of tert-butyl 4-aminopiperidine-1-carboxylate in 1 ml of abs. DMF with a total of 15.06 mg.(0.12 mmol) of Hünig's base and 13.29 mg (0.03 mmol) of HATU.

Yield: 14 mg (55% of theory)

LC-MS (Method 12): Rₜ=2.24 min

MS (EI): m/z=868 (M+H)⁺

### Example 70A

### tert-Butyl{3-[(8S,11S,14S)-14-[(tert-butoxycarbonyl)amino]-8-[({2-(tert-butoxy-carbonyl)amino]ethyl}amino)carbonyl]-5,17-dihydroxy-9-methyl-10,13-dioxo-9,12-diazatricyclo[14.3.1.1^{2,6}]henicosa-1(20),2(21),3,5,16,18-hexaen-11-yl]propyl}carbamate

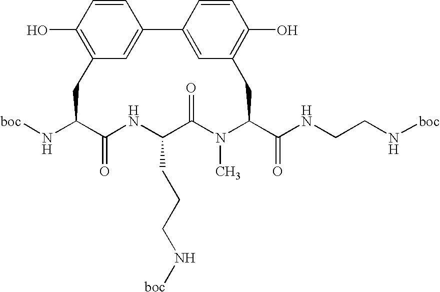

Preparation takes place in analogy to Example 68A from 10 mg (0.01 mmol) of the compound from Example 51A and 2.87 mg (0.02 mmol) of tert-butyl-(2-aminoethyl)carbamate in 1 ml of abs. DMF with a total of 7.71 mg (0.06 mmol) of Hünig's base and 4.43 mg (0.013 mmol) of HATU.

Yield: 3.5 mg (29% of theory)

LC-MS (Method 17): Rₜ=2.37 min

MS (EI): m/z=812 (M+H)⁺

### Example 71A

### Methyl (2Z)-3-[2-(benzyloxy)-5-(4,4,5,5-tetramethyl-1,3,2-dioxaborolan-2-yl)phenyl]-2-[(tert-butoxycarbonyl)amino]acrylate

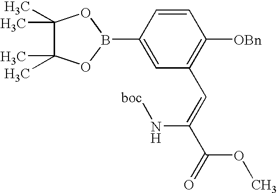

Preparation takes place in analogy to Example 8A from 1.0 g (2.16 mmol) of the compound from Example 54A, 0.63 g (2.5 mmol) of 4,4,4′,4′,5,5,5′,5′-octamethyl-2,2′-bi-1,3,2-dioxaborolane, 0.64 g (6.50 mmol) of potassium acetate and 0.063 g (0.087 mmol, 0.04 equivalent) of bis(diphenylphosphino)ferrocenepalladium(II) chloride in 14 ml of dimethyl sulphoxide.

Yield: 0.832 g (76% of theory)

LC-MS (Method 12): Rₜ=2.96 min

MS (EI): m/z=510 (M+H)⁺

### Example 72A

### tert-Butyl 4-[({[(8S,11S,14S)-14-[(tert-butoxycarbonyl)amino]11-{3-[(tert-butoxycarbonyl)amino]propyl}-5,17-dihydroxy-10,13-dioxo-9,12-diazatricyclo-[14.3.1.1^{2.6}]henicosa-1(20),2(21),3,5,16,18-hexaen-8-yl]carbonyl}amino)methyl]-piperidine-1-carboxylate

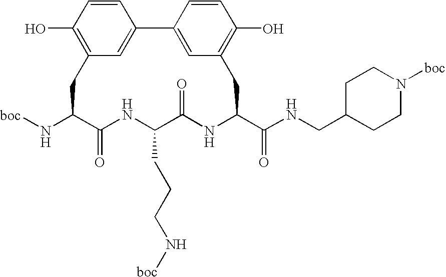

Preparation takes place in analogy to Example 68A from 15 mg (0.02 mmol) of the compound from Example 29A and 5.87 mg (0.03 mmol) of tert-butyl 4-(aminomethyl)piperidine-1-carboxylate in 1 ml of abs. DMF with a total of 10.33 mg (0.01 ml; 0.08 mmol) of Hünig's base and 10.42 mg (0.03 mmol) of HATU.

Yield: 8 mg (38% of theory)

LC-MS (Method 12): Rₜ=2.27 min

MS (EI): m/z=852 (M+H)⁺

### Example 73A

### tert-Butyl 4-({[(8S,11S,14S)-14-[(tert-butoxycarbonyl)amino]-11-{3-[(tert-butcarbonyl)amino]propyl}-5,17-dihydroxy-10,13-dioxo-9,12-diazatricyclo[14.3.1.1^{2,6}]-henicosa-1(20),2(21),3,5,16,18-hexaen-8-yl]carbonyl}amino)piperidine-1-carboxylate

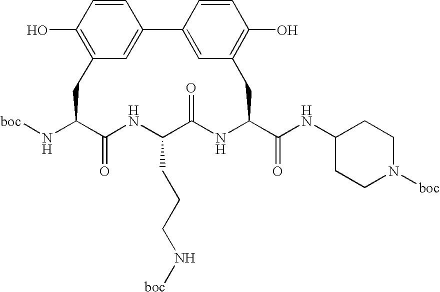

Preparation takes place in analogy to Example 68A from 15 mg (0.02 mmol) of the compound from Example 29A and 5.49 mg (0.03 mmol) of tert-butyl-4-aminopiperidine-1-carboxylate in 1 ml of abs. DMF with a total of 10.33 mg (0.01 ml; 0.08 mmol) of Hünig's base and 10.42 mg (0.03 mmol) of HATU.

Yield: 12 mg (56% of theory)

LC-MS (Method 18): Rₜ=2.40 min

MS (EI): m/z=838 (M+H)⁺

### Example 74A

### tert-Butyl{3-[(8S,11S,14S)-14-[(tert-butoxycarbonyl)amino]-8-[({2-[(tert-butoxy-carbonyl)amino]ethyl}amino)carbonyl]-5,17-dihydroxy-10,13-dioxo-9,12-diazatricyclo[14.3.1.1^{2,6}]henicosa-1(20),2(21),3,5,16,18-hexaen-11-yl]propyl}carbamate

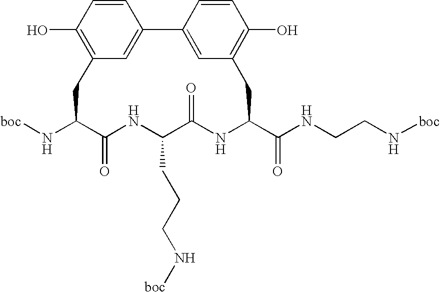

620 mg (0.9 mmol) of the compound from Example 29A and 244.3 mg (1.52 mmol) of tert-butyl(2-aminoethyl)carbamate are dissolved in 10.5 ml of abs. DMF and cooled in an ice bath and, while stirring, 292.3 mg (1.52 mmol) of EDC and 40 mg (0.3 mmol) of HOBt are added. The reaction mixture is allowed to warm to room temperature and, after 2 h, the product is precipitated by adding (vigorous stirring) 200 ml of water. After stirring for 30 min, the precipitate is filtered off. The product is dried under high vacuum.

Yield: 675 mg (85% of theory)

LC-MS (Method 12): Rₜ=2.12 min

MS (EI): m/z=798 (M+H)⁺

### Example 75A

### tert-Butyl{3-[(8S,11S,14S)-14-[(tert-butoxycarbonyl)amino]-8-[({3-[(tert-butoxy-carbonyl)amino]-2-hydroxypropyl}amino)carbonyl]-5,17-dihydroxy-10,13-dioxo-9,12-diazatricyclo[14.3.1.1^{2,6}]henicosa-1(20),2(21),3,5,16,18-hexaen-11-yl]propyl}carbamate

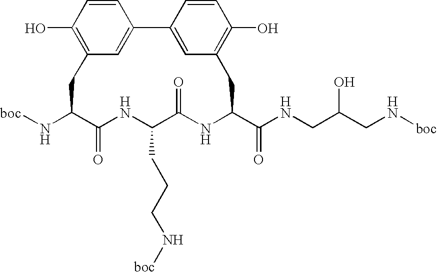

Preparation takes place in analogy to Example 68A from 15 mg (0.02 mmol) of the compound from Example 29A and 5.21 mg (0.03 mmol) of tert-butyl (3-amino-2-hydroxypropyl)carbamate in 1 ml of abs. DMF with a total of 10.33 mg (0.01 ml; 0.08 mmol) of Hünig's base and 10.42 mg (0.03 mmol) of HATU.

Yield: 10 mg (53% of theory)

LC-MS (Method 19): Rₜ=2.23 min

MS (EI): m/z=828 (M+H)⁺

### Example 76A

### tert-Butyl{3-[(8S,11S,14S)-14-[(tert-butoxycarbonyl)amino]-8-[({3-[(tert-butoxy-carbonyl)amino]propyl}amino)carbonyl]-5,17-dihydroxy-10,13-dioxo-9,12-diazatricyclo[14.3.1.1^{2,6}]henicosa-1(20),2(21),3,5,16,18-hexaen-11-yl]propyl}carbamate

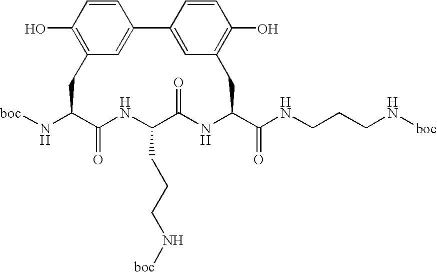

Preparation takes place in analogy to Example 68A from 15 mg (0.02 mmol) of the compound from Example 29A and 4.78 mg (0.03 mmol) of tert-butyl (3-aminopropyl)carbamate in 1 ml of abs. DMF with a total of 10.33 mg (0.01 ml; 0.08 mmol) of Hünig's base and 10.42 mg (0.03 mmol) of HATU.

Yield: 7.2 mg (37% of theory)

LC-MS (Method 12); Rₜ=2.16 min

MS (EI): m/z=812 (M+H)⁺

### Example 77A

### tert-Butyl[2-({[(8S,11S,14S)-14-[(tert-butoxycarbonyl)amino]-11-{3-[(tert-butoxy-carbonyl)amino]propyl}-5,17-dihydroxy-10,13-dioxo-9,12-diazatricyclo[14.3.1.1^{2,6}]-henicosa-1(20),2(21),3,5,16,18-hexaen-8-yl]carbonyl}amino)ethyl]methylcarbamate

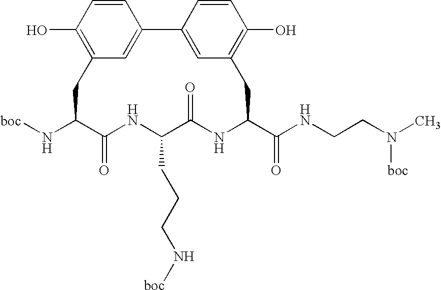

Preparation takes place in analogy to Example 68A from 15 mg (0.02 mmol) of the compound from Example 29A and 4.78 mg (0.03 mmol) of tert-butyl (3-aminopropyl)methylcarbamate in 1 ml of abs. DMF with a total of 10.33 mg (0.01 ml; 0.08 mmol) of Hünig's base and 10.42 mg (0.03 mmol) of HATU.

Yield: 5.5 mg (29% of theory)

LC-MS (Method 12): Rₜ=2.18 min

MS (EI): m/z=812 (M+H)⁺

### Example 78A

### 2-(Trimethylsilyl)ethyl(2Z)-2-{[(benzyloxy)carbonyl]amino}-3-(4,4′-bis(benzyl-oxy)-3′-{(1Z)-2-[(tert-butoxycarbonyl)amino]-3-methoxy-3-oxoprop-1-en-1-yl}biphenyl-3-yl)acrylate

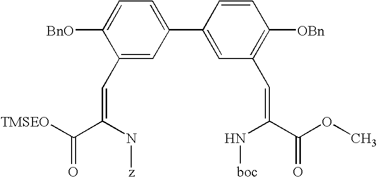

Preparation takes place in analogy to Example 12A from 0.42 g (0.82 mmol) of the compound from Example 71A, 0.48 g (0.82 mmol) of the compound from Example 49A, 0.54 g (1.65 mmol) of caesium carbonate and 0.024 g (0.033 mmol, 0.04 equivalent) of bis(diphenylphosphino)ferrocenepalladium(II) chloride in 12 ml of DMF.

Yield: 0.47 g (64% of theory)

HPLC (Method 16): Rₜ=6.57 min

MS (EI): m/z=886 (M+H)⁺

### Example 79A

### Benzyl((2R)-3-[(8S,11S,14S)-5,17-bis(benzyloxy)-14-{[(benzyloxy)carbonyl]-amino}-8-[({3-[(tert-butoxycarbonyl)amino]-2-hydroxypropyl}amino)carbonyl]-10,13-dioxo-9,12-diazatricyclo[14.3.1.1^{2,6}]henicosa-1(20),2(21),3,5,16,18-hexaen-11-yl]-2-{[tert-butyl(dimethyl)silyl]oxy}propyl)carbamate

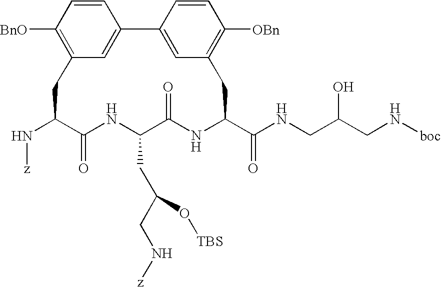

Preparation takes place in analogy to Example 68A from 60 mg (0.06 mmol) of the compound from Example 67A and 12.1 mg (0.06 mmol) of tert-butyl (3-amino-2-hydroxypropyl)carbamate in 4 ml of abs. DMF with a total of 60 mg (0.08 ml; 0.46 mmol) of Hünig's base and 27.6 mg (0.07 mmol) of HATU.

Yield: 69 mg (98% of theory)

LC-MS (Method 17): Rₜ=3.43 min

MS (EI): m/z=1207 (M+H)⁺

### Example 80A

### tert-Butyl[3-({[(8S,11S,14S)-14-amino-11-((2R)-3-amino-2-{[tert-butyl(dimethyl)-silyl]oxy}propyl)-5,17-dihydroxy-10,13-dioxo-9,12-diazatricyclo[14.3.1.1^{2,6}]-henicosa-1(20),2(21),3,5,16,18-hexaen-8-yl]carbonyl}amino)-2-hydroxypropyl]carbamate diacetate

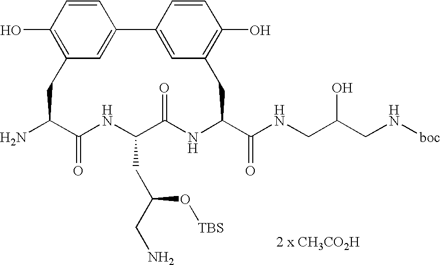

69 mg (0.06 mmol) of the compound from Example 79A are dissolved in 30 ml of glacial acetic acid/water/ethanol=4/1/1 and, after addition of 20 mg of Pd/C (10%) catalyst, hydrogenated with hydrogen at RT. After the catalyst has been removed by filtration, the filtrate is evaporated to dryness in vacuo.

Yield: 50 mg (quantitative)

LC-MS (Method 17): Rₜ=1.58 min

MS (EI): m/z 879 (M+H)⁺

### Example 81A

### tert-Butyl{(2R)-3-[(8S,11S,14S)-14-[(tert-butoxycarbonyl)amino]-8-[({2-[(tert-butoxycarbonyl)amino]ethyl}amino)carbonyl]-5,17-dihydroxy-10,13-dioxo-9,12-diazatricyclo[14.3.1.1^{2,6}]henicosa-1(20),2(21),3,5,16,18-hexaen-11-yl]-2-hydroxypropyl}carbamate

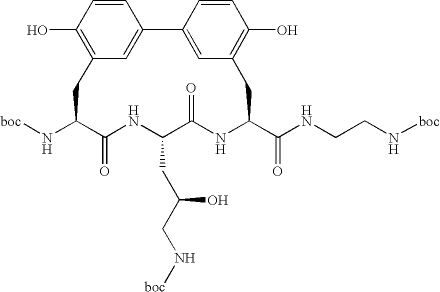

Preparation takes place in analogy to Example 68A from 35 mg (0.03 mmol) of the compound from Example 65A and 16.3 mg (0.1 mmol) of tert-butyl(2-aminoethyl)carbamate in 3 ml of abs. DMF with a total of 15.3 mg (0.02 ml; 0.12 mmol) of Hünig's base and 19.3 mg (0.05 mmol) of HATU.

Yield: 8 mg (29% of theory)

LC-MS (Method 18): Rₜ=3.05 min

MS (EI): m/z=1015 (M+H)⁺

### Example 82A

### tert-Butyl 2-[2-({[(8S,11S,14S)-5,17-bis(benzyloxy)-14-{[(benzyloxy)carbonyl]-amino}-11-(3-{[(benzyloxy)carbonyl]amino}propyl)-9-methyl-10,13-dioxo-9,12-diazatricyclo[14.3.1.1^{2,6}]henicosa-1(20),2(21),3,5,16,18-hexaen-8-yl]carbonyl}amino)ethyl]piperidine-1-carboxylate

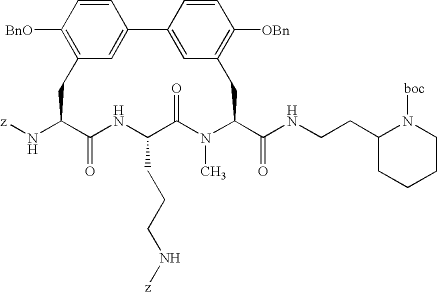

Preparation takes place in analogy to Example 68A from 45 mg (0.05 mmol) of the compound from Example 48A and 12.3 mg (0.05 mmol) of tert-butyl 2-(2-aminoethyl)piperidine-1-carboxylate in 5 ml of abs. DMF with a total of 50.6 mg (0.39 mmol) of Hünig's base and 23.3 mg (0.06 mmol) of HATU.

Yield: 46 mg (83% of theory)

LC-MS (Method 12): Rₜ=3.26 min

MS (EI): m/z=1129 (M+H)⁺

### Example 83A

### tert-Butyl 2-[({[(8S,11S,14S)-5,17-bis(benzyloxy)-14-{[(benzyloxy)carbonyl]-amino}-11-(3-{[benzyloxy)carbonyx]amino}propyl)-9-methyl-10,13-dioxo-9,12-diazatricyclo[14.3.1.1^{2,6}]henicosa-1(20),2(21),3,5,16,18-hexaen-8-yl]carbonyl}amino)methyl]piperidine-1-carboxylate

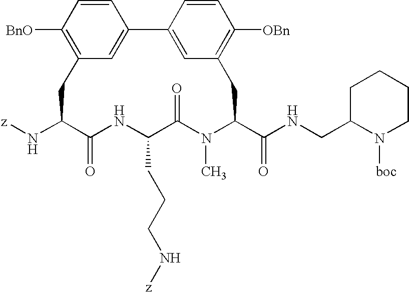

Preparation takes place in analogy to Example 68A from 45 mg (0.05 mmol) of the compound from Example 48A and 11.5 mg (0.05 mmol) of tert-butyl 2-(aminomethyl)piperidine-1-carboxylate in 5 ml of abs. DMF with a total of 50.6 mg (0.39 mmol) of Hünig's base and 23.3 mg (0.06 mmol) of HATU.

Yield: 48 mg (88% of theory)

LC-MS (Method 12): Rₜ=3.22 min

MS (EI): m/z=1115 (M+H)⁺

### Example 84A

### 2-(Benzyloxy)-N-(tert-butoxycarbonyl)-5-iodo-N-ethyl-L-phenylalanine

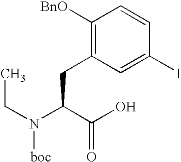

Under an argon atmosphere, 1.0 g (2.01 mmol) of the compound from Example (−)-6A are dissolved in 40 ml of TBF, mixed with 241 mg (6.03 mmol) of sodium hydride (60% dispersion in mineral oil), 1.0 g (6.03 mmol) of potassium iodide and 1.29 ml (2509 mg; 16.1 mmol) of ethyl iodide and stirred at room temperature overnight. The mixture is concentrated in vacuo. The crude product is taken up in ethyl acetate, and the organic phase is washed several times with water, dried over sodium sulphate and concentrated in vacuo. The crude product is purified by RP-HPLC chromatography (mobile phase acetonitrile/water gradient).

Yield: 470 mg (44% of theory)

LC-MS (Method 12): Rₜ=2.79 min

MS (EI): m/z=526 (M+H)⁺

### Example 85A

### Benzyl 2-(benzyloxy)-N-(tert-butoxycarbonyl)-5-iodo-N-ethyl-L-phenylalaninate

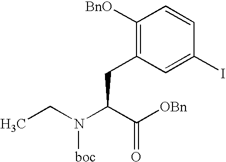

Preparation takes place in analogy to Example 7A from 420 mg (0.68 mmol) of the compound from Example 84A, 9.77 mg (0.08 mmol) of DMAP, 173 mg (1.6 mmol) of benzyl alcohol and 184 mg (0.96 mmol) of EDC in 8 ml of acetonitrile.

Yield: 375 mg (76% of theory)

LC-MS (Method 12): Rₜ=3.26 min

MS (EI): m/z=616 (M+H)⁺

¹H-NMR (300 MHz, CDCl₃): δ=0.80 (m_{c}, 3H), 1.4 (m_{c}, 9H) 2.75 (m_{c}, 1H), 3.07 (m_{c}, 1H), 3.22 (m_{c}, 1H), 3.47 (m_{c}, 1H), 4.23 (m_{c}, 1H), 5.06 (s, 2H), 5.15 (m_{c}, 2H), 6.65 (d, 1H), 7.25-7.5 (m, 12H).

### Example 86A

### 2-(Trimethylsilyl)ethyl 2(S)-benzyloxycarbonylamino-3-[4,4′-bisbenzyloxy-3′-(2(S)-benzyloxycarbonyl-(2-tert-butoxycarbonyl-2-ethyl)aminoethyl)biphenyl-3-yl]-propionate

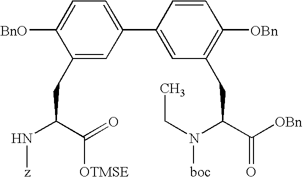

Preparation takes place in analogy to Example 12A from 343 mg (0.54 mmol) of the compound from Example 57A, 334 mg (0.54 mmol) of the compound from Example 85A, 354 mg (1.09 mmol) of caesium carbonate and 40 mg (0.05 mmol) of bis(diphenylphosphino)ferrocenepalladium(II) chloride in 8 ml of DMF under an argon atmosphere.

Yield: 216 mg (40% of theory)

LC-MS (Method 12): Rₜ=3.54 min

MS (EI): m/z=893 (M-boc+H)⁺

### Example 87A

### 2-(Trimethylsilyl)ethyl 2(S)-benzyloxycarbonylamino-3-[4,4′-bisbenzyloxy-3′-(2(S)-benzyloxycarbonyl-2-ethylaminoethylbiphenyl-3-yl]propionate hydrochloride

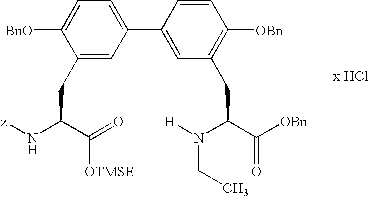

Preparation takes place in analogy to Example 15A from 210 mg (0.211 mmol) of the compound from Example 86A and 15 ml of a 4N hydrogen chloride/dioxane solution in 4 ml of dioxane.

Yield: quantitative

LC-MS (Method 12): Rₜ=3.01 min

MS (EI): m/z=893 (M-HCl+H)⁺

Examples 88A to 92A listed in the following table are prepared in analogy to the methods detailed above from the appropriate starting compounds:

| Ex. | | Prepared to | |
|---|---|---|---|
| No. | Structure | analogy to | Analytical data |
| 88A | 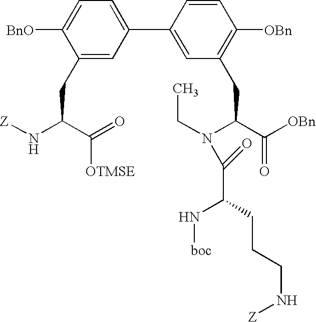 | 16A with N⁵-[(ben- zyloxy)- carbonyl]-N²- (tert-butoxy- carbonyl)-L- ornithine | LC-MS (Method 17): Rₜ = 3.63 min. MS (EI): m/z = 1241 (M + H)⁺ |
| 89A | 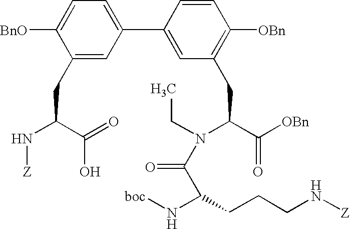 | 17A | LC-MS (Method 17): Rₜ = 3.38 min. MS (EI): m/z = 1149 (M + H)⁺ |
| 90A | 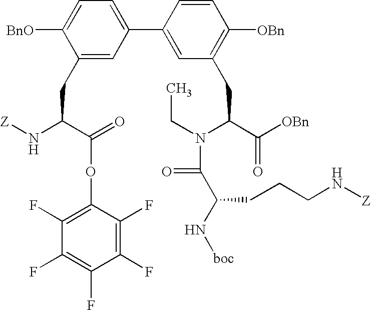 | 18A | LC-MS (Method 17): Rₜ = 3.58 min. MS (EI): m/z = 1315 (M + H)⁺ |
| 91A | 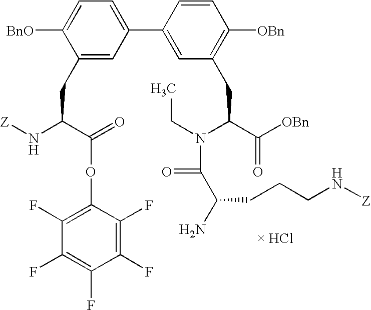 | 26A | |
| 92A | 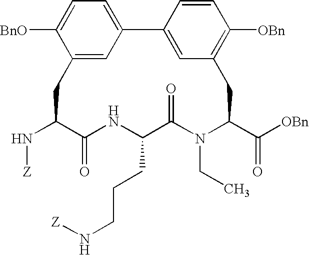 | 39A | LC-MS (Method 17): Rₜ = 3.39 min. MS (EI): m/z = 931 (M + H)⁺ |

### Example 93A

### Benzyl{3-[(8S,11S,14S)-8-{[(2-aminoethyl)amino]carbonyl}-5,17-bis(benzyloxy)-14-{[(benzyloxy)carbonyl]amino}-9-ethyl-10,13-dioxo-9,12-diazatricyclo-[14.3.1.1^{2,6}]henicosa-1(20),2(21),3,5,16,18-hexaen-11-yl]propyl}carbamate

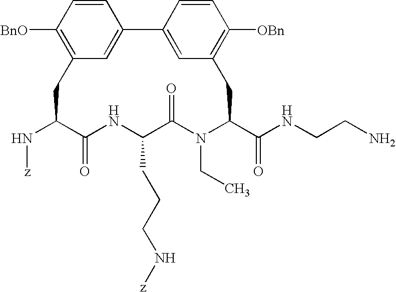

16.5 mg (0.02 mmol) of the compound from Example 92A are dissolved in 270 μl of diethylamine. Addition of 0.1 mg (10 mol %) of potassium cyanide is followed by stirring at RT for 36 h. Ethyl acetate is then added, and the organic phase is washed with saturated sodium bicarbonate solution and water, dried over sodium sulphate and concentrated in vacuo.

Yield: 17.5 mg (88% of theory)

LC-MS (Method 17): Rₜ=2.33 min

MS (EI): m/z=975 (M+H)⁺

Examples 94A to 108A listed in the following table are prepared in analogy to the methods detailed above from the appropriate starting compounds:

| Example | | Prepared in | |
|---|---|---|---|
| No. | Structure | analogy to | Analytical data |
| 94A | 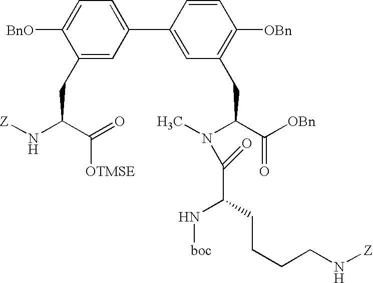 | 16A with N⁶- [(benzyloxy)- carbonyl]-N²-(tert- butoxycarbonyl)- L-lysine | LC-MS (Method 17): Rₜ =3.63 min. MS (EI): m/z = 1241 (M + H)⁺ |
| 95A | 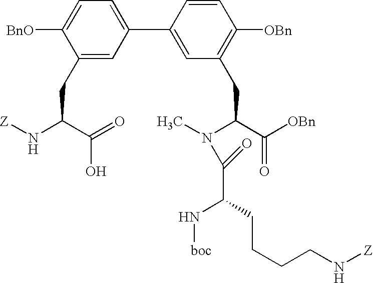 | 17A | LC-MS (Method 19): Rₜ = 3.40 min. MS (EI): m/z = 1141 (M + H)⁺ |
| 96A | 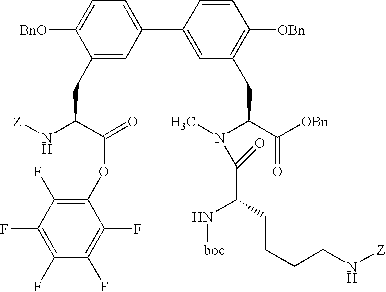 | 18A | LC-MS (Method 12): Rₜ =3.42 min. MS (EI): m/z = 1307 (M + H)⁺ |
| 97A | 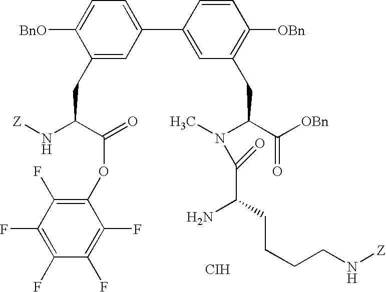 | 26A | |
| 98A | 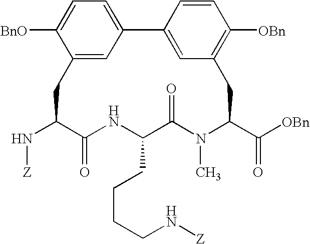 | 39A | LC-MS (Method 12): Rₜ =3.27 min. MS (EI): m/z = 1023 (M + H)⁺ |
| 99A | 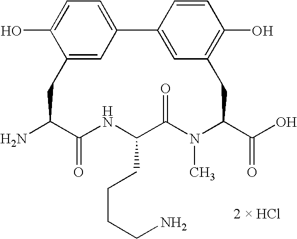 | 28A | LC-MS (Method 20): Rₜ =2.43 min. MS (EI): m/z = 485 (M-2HCl + H)⁺ |
| 100A | 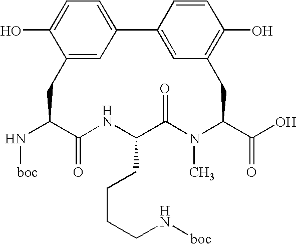 | 29A | LC-MS (Method 20): Rₜ =3.26 min. MS (EI): m/z = 685 (M + H)⁺ |
| 101A | 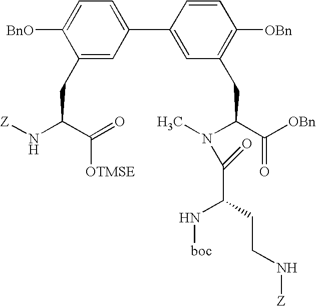 | 16A with (2S)-4- {[(benzyloxy)- carbonyl]amino}- 2-[(tert- butoxycarbonyl)- amino]butanoic acid | LC-MS (Method 17): Rₜ =3.65 min. MS (EI): m/z = 1213 (M + H)⁺ |
| 102A | 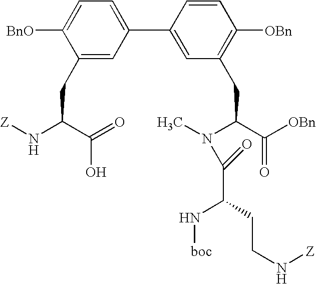 | 17A | LC-MS (Method 19): Rₜ =3.33 min. MS (EI): m/z 1113 (M + H)⁺ |
| 103A | 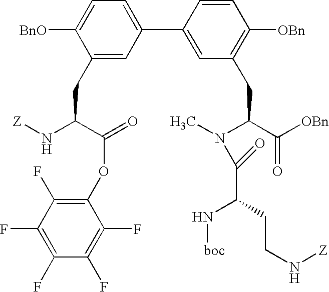 | 18A | LC-MS (Method 19): Rₜ =3.52 min. MS (EI): m/z = 1279 (M + H)⁺ |
| 104A | 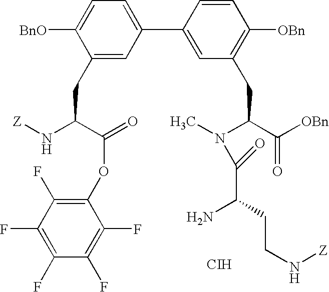 | 26A | LC-MS (Method 12): Rₜ =1.83 min. MS (EI): m/z = 1179 (M-HCl + H)⁺ |
| 105A | 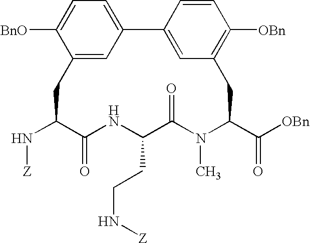 | 39A | LC-MS (Method 17): Rₜ =3.40 min. MS (EI): m/z = 995 (M + H)⁺ |
| 106A | 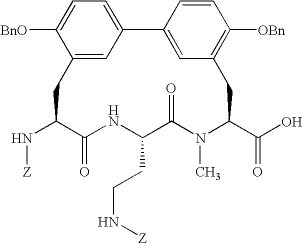 | 41A | LC-MS (Method 12): Rₜ =3.0 min. MS (EI): m/z = 905 (M + H)⁺ |
| 107A | 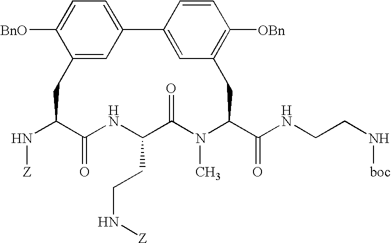 | 68A | LC-MS (Method 12): Rₜ =3.13 min. MS (EI): m/z = 1047 (M + H)⁺ |
| 108A | 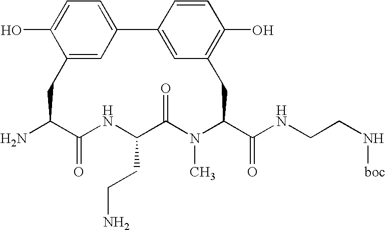 | 28A | LC-MS (Method 20): Rₜ =2.62 min. MS (EI): m/z = 598 (M + H)⁺ |

### Example 109A

### tert-Butyl[2-({[(8S,11S,14S)-14-[(tert-butoxycarbonyl)amino]-11-{4-[(tert-butoxycarbonyl)amino]butyl}-5,17-dihydroxy-9-methyl-10,13-dioxo-9,12-diazatricyclo[14.3.1.1^{2,6}]henicosa-1(20),2(21),3,5,16,18-hexaen-8-yl]carbonyl}amino)ethyl]carbamate

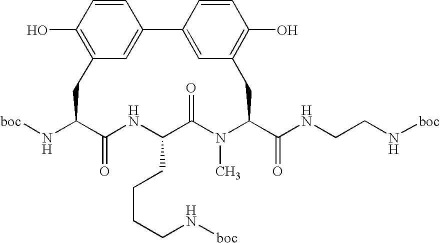

30 mg (0.04 mmol) of the compound from Example 100A and 11.9 mg (0.07 mmol) of tert-butyl(2-aminoethyl)carbamate are dissolved in 2 ml of dimethylformamide under argon. Then, at 0° C. (ice bath), 14.3 mg (0.07 mmol) of EDC and 2 mg (0.01 mmol) of HOBt are added. The mixture is slowly warmed to RT and stirred at RT for 12 h. The solution is concentrated in vacuo, and the residue is stirred with water. The remaining solid is filtered off with suction and dried under high vacuum.

Yield: 31.1 mg (64% of theory)

LC-MS (Method 20): Rₜ=3.55 min

MS (EI): m/z=827 (M+H)⁺

Examples 110A to 119A listed in the following table are prepared in analogy to the methods detailed above from the appropriate starting compounds:

| Example | | Prepared in | |
|---|---|---|---|
| No. | Structure | analogy to | Analytical data |
| 110A | 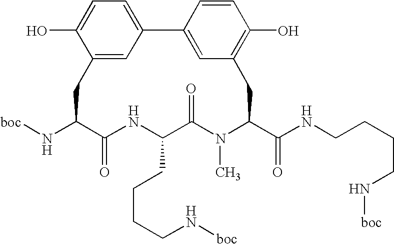 | 109A | LC-MS (Method 20): Rₜ = 3.60 min. MS (EI): m/z = 855 (M + H)⁺ |
| 111A | 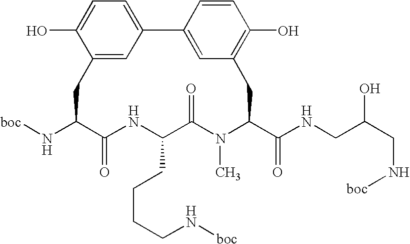 | 109A | LC-MS (Method 12): Rₜ = 2.15 min. MS (EI): m/z = 857 (M + H)⁺ |
| 112A | 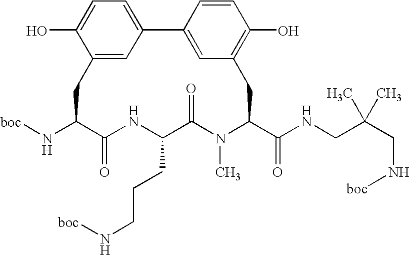 | 109A | LC-MS (Method 17): Rₜ = 2.55 min. MS (EI): m/z = 855 (M + H)⁺ |
| 113A | 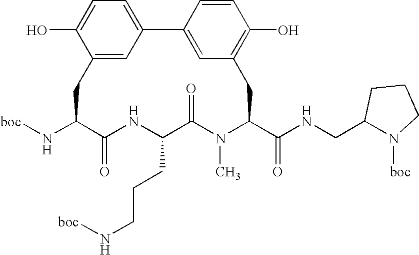 | 109A | LC-MS (Method 17): Rₜ = 2.52 min. MS (EI): m/z = 853 (M + H)⁺ |
| 114A | 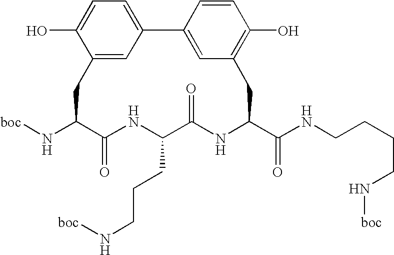 | 109A | LC-MS (Method 12): Rₜ = 2.17 min. MS (EI): m/z = 827 (M + H)⁺ |
| 115A | 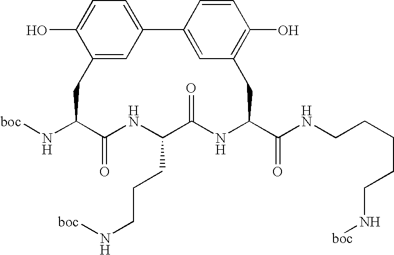 | 109A | LC-MS (Method 19): Rₜ = 2.43 min. MS (EI): m/z = 841 (M + H)⁺ |
| 116A | 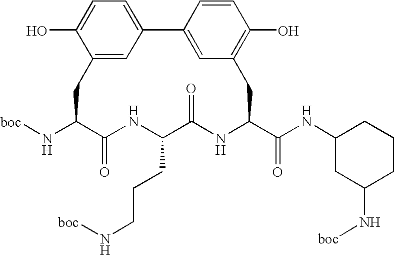 | 109A | LC-MS (Method 19): Rₜ = 2.46 min. MS (EI): m/z = 853 (M + H)⁺ |
| 117A | 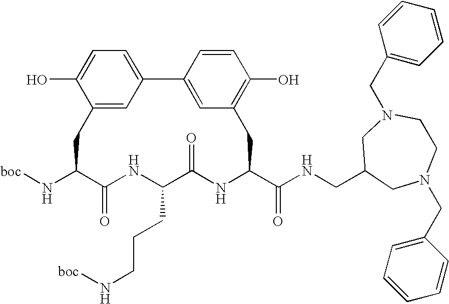 | 109A | LC-MS (Method 20): Rₜ = 2.12 min. MS (EI): m/z = 948 (M + H)⁺ |
| 118A | 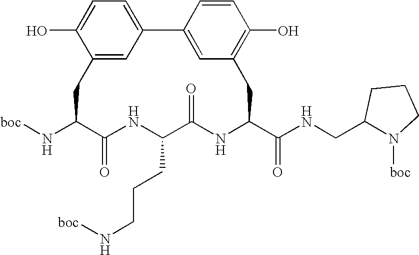 | 109A | LC-MS (Method 12): Rₜ = 2.33 min. MS (EI): m/z = 839 (M + H)⁺ |
| 119A | 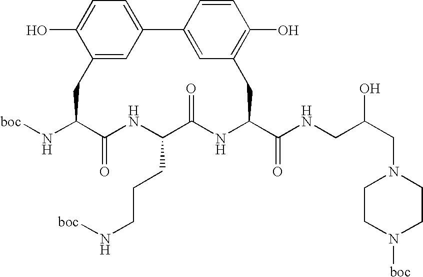 | 109A | LC-MS (Method 19): Rₜ = 1.95 min. MS (EI): m/z = 898 (M + H)⁺ |

### Example 120A

### tert-Butyl{3-[(8S,11S,14S)-14-[(tert-butoxycarbonyl)amino]-8-[(1,4-diazepan-6-ylamino)carbonyl]-5,17-dihydroxy-10,13-dioxo-9,12-diazatricyclo[14.3.1.1^{2,6}]-henicosa-1(20),2(21),3,5,16,18-hexaen-11-yl]propyl}carbamate dihydrochloride

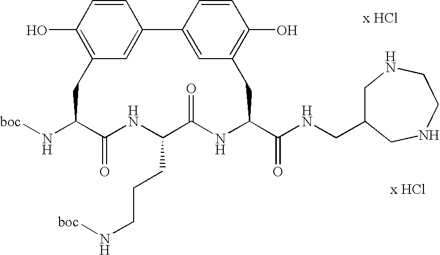

19.9 mg (0.021 mmol) of the compound from Example 117A are suspended in 4 ml of acetic acid/ethanol/water (4:1:1), mixed with 10 mg of Pd/C catalyst (10%) and hydrogenated under atmospheric pressure at RT for 1 h. The catalyst is filtered off through a membrane filter, and the filtrate is evaporated to dryness in vacuo. 1 ml of 0.1N hydrochloric acid is added, and evaporation to dryness is repeated.

Yield: 12 mg (68% of theory)

LC-MS (Method 12): Rₜ=1.31 min

MS (EI): m/z=767 (M-2HCl+H)⁺

### Example 121A

### tert-Butyl{3-[(8S,11S,14S)-8-({[(1R,2R)-2-aminocyclohexyl]amino}carbonyl)-14-[(tert-butoxycarbonyl)amino]-5,17-dihydroxy-10,13-dioxo-9,12-diazatricyclo-[14.31.1^{2,6}]henicosa-1(20),2(21),3,5,16,18-hexaen-11-yl]propyl}carbamate

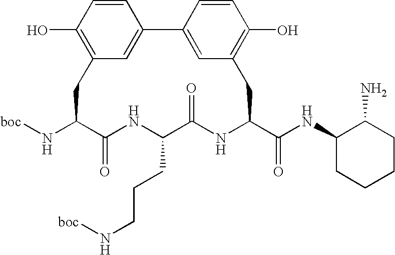

40 mg (0.060 mmol) of the compound from Example 29A are dissolved in 5 ml of dimethylformamide and cooled to 0° C. 34 mg (0.13 mmol) of 2-chloro-1,3-dimethyl-2-imidazolinium hexafluorophosphate (CIP) and 14 mg (0.12 mmol) of trans-1,2-DMAP and 0.050 ml (40 mg, 0.30 mmol) of diisopropylethylamine are added, the mixture is stirred at 0° C. for one hour and the crude solution is subsequently concentrated in vacuo. The residue is purified by HPLC (mobile phase acetonitrilelwater gradient).

Yield: 2 mg (4% of theory)

LC-MS (Method 20): Rₜ=3.27 min

MS (EI): m/z=753 (M+H)⁺

Examples 122A and 123A listed in the following table are prepared in analogy to the methods detailed above from the appropriate starting compounds:

| Example | | Prepared to | |
|---|---|---|---|
| No. | Structure | analogy to | Analytical data |
| 122A | 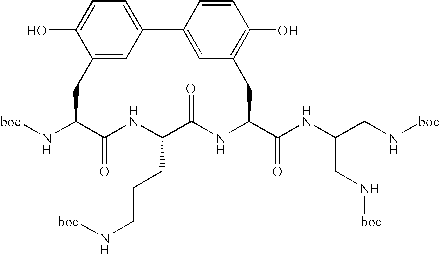 | 109A | LC-MS (Methode 12): Rₜ = 2.36 min. MS (EI): m/z = 928 (M + H)⁺ |
| 123A | 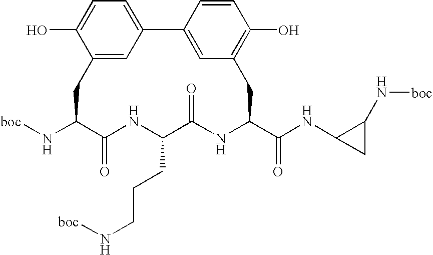 | 109A | LC-MS (Methode 17): Rₜ = 2.34 min. MS (EI): m/z = 811 (M + H)⁺ |

### Example 124A

### Benzyl{3-[(2-[(tert-butoxycarbonyl)amino]-1-{[(tert-butoxycarbonyl)amino]-methyl}ethyl)amino]propyl}carbamate

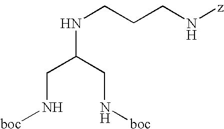

310 mg (1.07 mmol) of di-tert-butyl(2-aminopropane-1,3-diyl)biscarbamate and 222 mg (1.07 mmol) of benzyl (3-oxopropyl)carbamate are dissolved in 15 ml of dichloromethane. 334 mg (1.5 mmol) of sodium triacetoxyborohydride are added, and the mixture is stirred at room temperature overnight: The mixture is evaporated and the residue is purified by preparative HPLC.

Yield: 168 mg (38% of theory)

LC-MS (Method 12): Rₜ=1.76 min

MS (EI): m/z=481 (M+H)⁺

### Example 125A

### Benzyl{3-[(tert-butoxycarbonyl)(-2-[(tert-butoxycarbonyl)amino]-1-{[(tert-butoxy-carbonyl)amino]methyl}ethyl)amino]propyl}carbamate

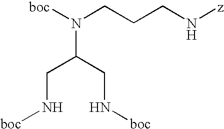

0.55 ml of a 10% strength triethylamine solution in acetonitrile and 154 mg (0.70 mmol) of di-tert-butyl dicarbonate are added to a solution of 168 mg (0.35 mmol) of benzyl{3-[(2-[(tert-butoxycarbonyl)amino]-1-{[(tert-butoxycarbonyl)amino]methyl}ethyl)amino]propyl}carbamate (Example 124A) in 2 ml of acetonitrile. The reaction mixture is stirred at 60° C. for 6 hours. The solution is concentrated and the crude product is reacted without further purification.

Yield: quant.

LC-MS (Method 17): Rₜ=2.87 min

MS (EI): m/z=580 (M+H)⁺

### Example 126A

### Di-tert-butyl{2-[(3-aminopropyl)(tert-butoxycarbonyl)amino]propan-1,3-diyl}biscarbamate

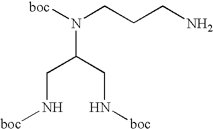

A solution of 190 mg (0.327 mmol) of benzyl {3-[(tert-butoxycarbonyl)(-2-[(tert-butoxycarbonyl)amino]-1-{[(tert-butoxycarbonyl)amino]methyl}ethyl)amino]-propyl}carbamate (Example 125A) in 50 ml of glacial acetic acid/water/ethanol (4/1/1) is hydrogenated after addition of 20 mg of palladium on activated carbon (10%) under atmospheric pressure at room temperature for 12 h. The mixture is filtered through kieselguhr, and the residue is washed with ethanol. The filtrate is evaporated to dryness in vacuo. The product is reacted without further purification.

Yield: quant.

LC-MS (Method 17): Rₜ=1.71 min

MS (EI): m/z=447 (M+H)⁺

Example 127A detailed in the following table is prepared in analogy to the method for Example 93A detailed above from the appropriate starting compounds:

| Ex. | Precursor of | | |
|---|---|---|---|
| No. | example | Structure | Analytical data |
| 127A | 27A and N-(3- aminopropyl)- propane-1,3- diamine | 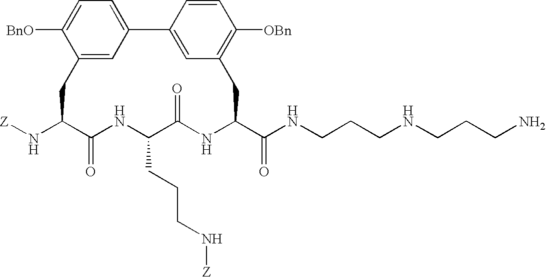 | HPLC (Method 1): Rₜ = 4.97 min. MS (EI): m/z = 1018 (M)⁺ |

Examples 128A to 134A listed in the following table are prepared in analogy to the method of Example 109A from the appropriate starting compounds:

| Ex. | Precursor of | | |
|---|---|---|---|
| No. | example | Structure | Analytical data |
| 128A | 29A | 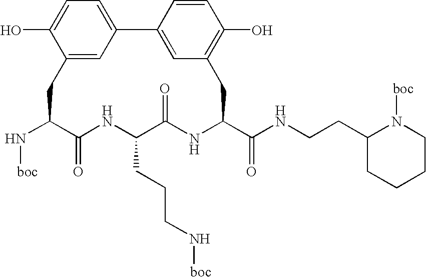 | LC-MS (Method 12): Rₜ =2.42 min. MS (EI): m/z = 867 (M + H)⁺. |
| 129A | 29A | 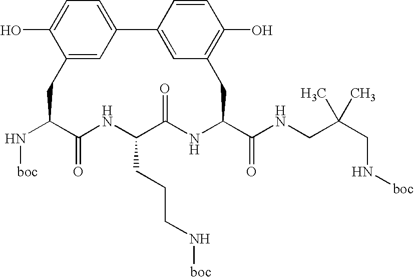 | LC-MS (Method 17): Rₜ =2.49 min. MS (EI): m/z = 841 (M + H)⁺. |
| 130A | 29A | 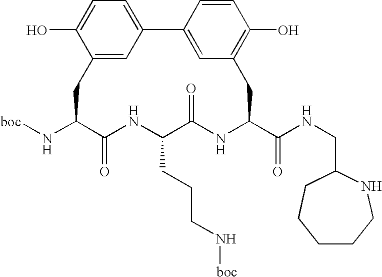 | LC-MS (Method 17): Rₜ =1.84 min. MS (EI): m/z = 767 (M + H)⁺ |
| 131A | 29A | 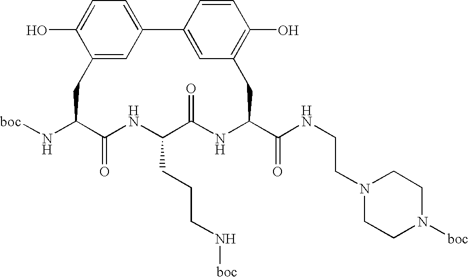 | LC-MS (Method 19): Rₜ =2.01 min. MS (EI): m/z = 867 (M + H)⁺ |
| 132A | 29A | 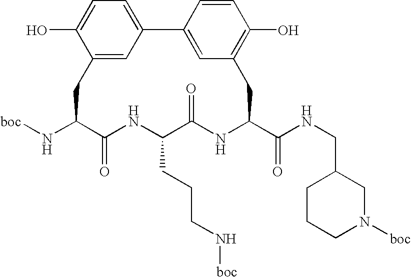 | LC-MS (Method 19): Rₜ =2.46 min. MS (EI): m/z = 851 (M − H)⁺ |
| 133A | 29A | 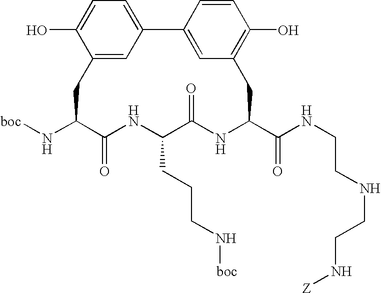 | LC-MS (Method 17): Rₜ =1.84 min. MS (EI): m/z = 876 (M + H)⁺ |
| 134A | 29A and 126A | 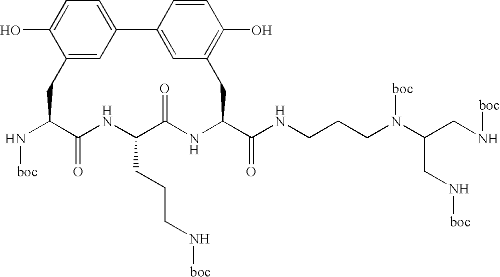 | LC-MS (Method 19): Rₜ =2.72 min. MS (EI): m/z = 1085 (M + H)⁺ |

Example 135A detailed in the following table is prepared in analogy to the method of Example 120A from the appropriate starting compounds:

| | Precursor | | |
|---|---|---|---|
| Example | of | | |
| No. | example | Structure | Analytical data |
| 135A | 133A | 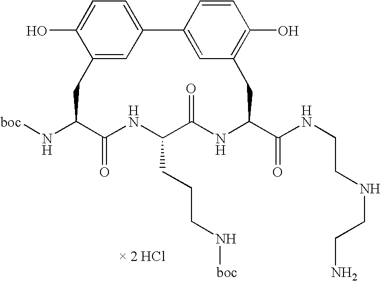 | LC-MS (Method 12): Rₜ = 1.37 min. MS (EI): m/z = 741 (M-2HCl + H)⁺ |

### Example 136A

### Benzyl{3-[(8S,11S,14S)-5,17-bis(benzyloxy)-14-{[(benzyloxy)carbonyl]amino}-8-[({2-[bis(2-aminoethyl)amino]ethyl}amino)carbonyl]-10,13-dioxo-9,12-diaza-tricyclo[14.3.1.1^{2,6}]henicosa-1(20),2(21),3,5,16,18-hexaen-1-yl]propyl}carbamate

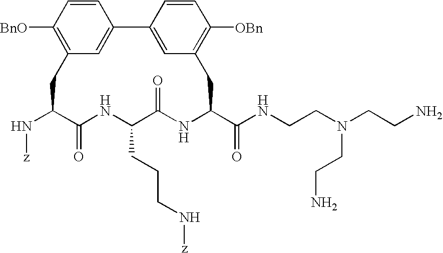

20 mg (0.02 mmol) of benzyl 5,17-bisbenzyloxy-14(S)-benzyloxycarbonylamino-11(S)-(3-benzyloxycarbonylaminopropyl)-10,13-dioxo-9,12-diazatricyclo-[14.3.1.1^{2,6}]henicosa-1(19),2,4,6(21),16)20),17-hexaene-8(S)-carboxylate (Example 27A) are dissolved in 489 mg (3.34 mmol) of tris(2-aminomethyl)amine, and 0.2 mg of potassium cyanide is added. The resulting suspension is dissolved by adding a few drops of dimethylformamide. The mixture is stirred at room temperature overnight, 10 ml of water are added, and the precipitate is filtered off. The crude product is obtained by drying in vacuo.

Yield: 10 mg (48% of theory)

LC-MS (Method 17): Rₜ=2.11 min

MS (EI): m/z=1034 (M+H)⁺

### Example 137A

### tert-Butyl[(1S)-4-(tert-butoxycarbonyl)amino]-1-(hydroxymethyl)butyl]carbamate

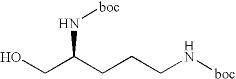

91 mg (0.90 mmol) of 4-methylmorpholine and 98 mg (0.90 mmol) of ethyl chloroformate are added to a solution of 300 mg (0.90 mmol) of N²,N⁵-bis(tert-butoxycarbonyl)-L-omithine in 10 ml of tetrahydrofuran at −10° C., and the mixture is stirred for 30 min. At this temperature, 1.81 ml (1.81 mmol) of a 1M solution of lithium aluminium hydride in tetrahydrofuran are slowly added dropwise. The mixture is slowly warmed to RT and stirred at RT for 12 h. While cooling in ice, 0.1 ml of water and 0.15 ml of 4.5% strength sodium hydroxide solution are cautiously added, and the mixture is stirred at RT for a further 3 h. The mixture is filtered, and the filtrate is concentrated in vacuo. The residue is dissolved in ethyl acetate, washed with water, dried over magnesium sulphate and again evaporated to dryness in vacuo. The product is reacted without further purification.

Yield: 239 mg (83% of theory)

MS (ESI): m/z=319 (M+H)⁺; 341 (M+Na)⁺

### Example 138A

### (2S)-2,5-Bis[(tert-butoxycarbonyl)amino]pentyl methanesulphonate

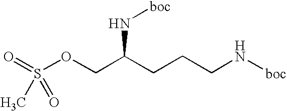

A solution of 240 mg (0.75 mmol) of tert-butyl [(1S)-4-[(tert-butoxycarbonyl)amino]-1-(hydroxymethyl)butyl]carbamate (Example 137A) in 20 ml of dichloromethane is mixed with 103 mg (0.90 mmol) of methanesulphonyl chloride and 0.21 ml (1.5 mmol) of triethylamine and stirred at RT for 16 h. It is diluted with dichloromethane and washed twice with 0.1N hydrochloric acid. The organic phase is dried over magnesium sulphate and evaporated to dryness in vacuo. The product is reacted without further purification.

Yield: 218 mg (73% of theory)

MS (ESI): m/z=419 (M+Na)⁺

### Example 139A

### tert-Butyl{(4S)-5-azido-4-[(tert-butoxycarbonyl)amino]pentyl}carbamate

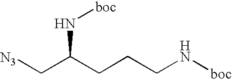

A solution of 218 mg (0.55 mmol) of (2S)-2,5-bis[(tert-butoxy-carbonyl)amino]pentylmethanesulphonate (Example 138A) in 15 ml of dimethylformamide is mixed with 36 mg (0.55 mmol) of sodium azide and stirred at 70° C. for 12 h. Most of the solvent is distilled off in vacuo, and the residue is diluted with ethyl acetate. It is washed several times with saturated sodium bicarbonate solution, dried over magnesium sulphate and evaporated to dryness in vacuo. The product is reacted without further purification.

Yield: 188 mg (99% of theory)

MS (ESI): m/z=344 (M+H)⁺

### Example 140A

### tert-Butyl{(4S)-5-amino-4-[(tert-butoxycarbonyl)amino]pentyl}carbamate

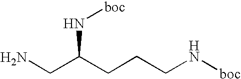

A solution of 188 mg (0.55 mmol) of tert-butyl{(4S)-5-azido4-[(tert-butoxycarbonyl)amino]pentyl}carbamate (Example 139A) in ethanol is hydrogenated after addition of 20 mg of palladium on activated carbon (10%) at RT under atmospheric pressure for 12 h. The mixture is filtered through kieselguhr, and the residue is washed with ethanol. The filtrate is evaporated to dryness in vacuo. The product is reacted without further purification.

Yield: 102 mg (59% of theory)

MS (ESI): m/z=318 (M+H)⁺; 340 (M+Na)⁺

### Example 141A

### Benzyl[(1S)-4-[(tert-butoxycarbonyl)amino]-1-(hydroxymethyl)butyl]carbamate

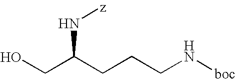

Preparation takes place in analogy to Example 137A from 570 mg (1.56 mmol) of N²-[(benzyloxy)carbonyl]-N⁵-(tert-butoxycarbonyl)-L-ornithine in 10 ml of tetrahydrofuran with 157 mg (1.56 mmol) of 4-methylmorpholine, 169 mg (1.56 mmol) of ethyl chloroformate and 3.11 ml (3.11 mmol) of a 1M solution of lithium aluminium hydride in tetrahydrofuran. The product is purified by preparative RP-HPLC (mobile phase water/acetonitrile gradient: 90:10→5:95).

Yield: 170 mg (31% of theory)

LC-MS (Method 12): Rₜ=1.88 min

MS (EI): m/z=353 (M+H)⁺

### Example 142A

### tert-Butyl[(4S)-4-amino-5-hydroxypentyl]carbamate

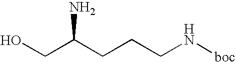

A solution of 169 mg (0.48 mmol) of benzyl[(1S)-4-[(tert-butoxycarbonyl)amino]-1-(hydroxymethyl)butyl]carbamate (Example 141A) in 50 ml of ethanol is hydrogenated after addition of 17 mg of palladium on activated carbon (10%) at RT under atmospheric pressure for 4 h. The mixture is filtered through kieselguhr, and the residue is washed with ethanol. The filtrate is evaporated to dryness in vacuo. The product is reacted without further purification.

Yield: 104 mg (99% of theory)

MS (DCI): m/z=219 (M+H)⁺

### Example 143A

### Benzyl[(1S)-3-[(tert-butoxycarbonyl)amino]-1-(hydroxymethyl)propyl]carbamate

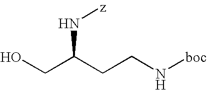

Preparation takes place in analogy to Example 137A from 300 mg (0.85 mmol) of (2S)-2-{[(benzyloxy)carbonyllanino}-4-[(tert-butoxycarbonyl)amino]butane-carboxylic acid in 10 ml of tetrahydrofuran with 86 mg (0.85 mmol) of 4-methylmorpholine, 92 mg (0.85 mmol) of ethyl chloroformate and 1.7 ml (1.70 mmol) of a 1M solution of lithium aluminium hydride in tetrahydrofuran. The product is reacted without further purification.

Yield: 229 mg (80% of theory)

LC-MS (Method 12): Rₜ=1.83 min

MS (EI): m/z=339 (M+H)⁺; 239 (M-C₅H₈O₂+H)⁺

### Example 144A

### tert-Butyl [(3S)-3-amino-4-hydroxybutyl]carbamate hydrochloride

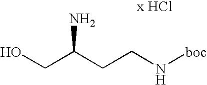

Preparation takes place in analogy to Example 142A from 229 mg (0.68 mmol) of benzyl [(1S)-3-[(tert-butoxycarbonyl)amino]-1-hydroxymethyl)propyl]carbamate (Example 143A) in 50 ml of ethanol with addition of 23 mg of palladium on activated carbon (10%). The crude product is stirred in 1 ml of IN hydrochloric acid and evaporated in vacuo, and dried to constant weight under high vacuum.

Yield: 183 mg (90% of theory)

MS (ESI): m/z=205 (M-HCl+H)⁺

### Example 145A

### tert-Butyl {(3S)-3-[(tert-butoxycarbonyl)amino]-4-hydroxybutyl}carbamate

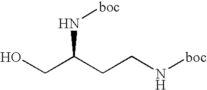

Preparation takes place in analogy to Example 137A from 300 mg (0.60 mmol) of (2S)-2,4-bis[(tert-butoxycarbonyl)amino]butanoic acid/N-cyclohexylcyclohexanamine (1:1) in 10 ml of tetrahydrofuran with 61 mg (0.60 mmol) of 4-methylmorpholine, 65 mg (0.60 mmol) of ethyl chloroformate and 1.2 ml (1.20 mmol) of a 1M solution of lithium aluminium hydride in tetrahydrofuran. The product is reacted without further purification.

Yield: 174 mg (95% of theory)

MS (ESI): m/z=305 (M+H)⁺

### Example 146A

### (2S)-2,4-Bis[(tert-butoxycarbonyl)amino]butyl methanesulphonate

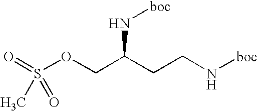

Preparation takes place in analogy to Example 138A from 250 mg (0.81 mmol) of tert-butyl {(3S)-3-[(tert-butoxycarbonyl)amino]4-hydroxybutyl}carbamate (Example 145A) in 20 ml of dichloromethane with 110 mg (0.97 mmol) of methanesulphonyl chloride and 0.23 ml (1.6 mmol) of triethylamine. The product is reacted without further purification.

Yield: 200 mg (64% of theory)

MS (ESI): m/z=383 (M+H)⁺; 400 (M+Na)⁺

### Example 147A

### tert-Butyl {(3S)-4-azido-3-[(tert-butoxycarbonyl)amino]butyl}carbamate

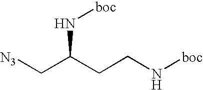

Preparation takes place in analogy to Example 139A from 200 mg (0.52 mmol) of (2S)-2,4-bis[(tert-butoxycarbonyl)amino]butyl methanesulphonate (Example 146A) in 15 ml of dimethylformamide with 34 mg (0.52 mmol) of sodium azide. The product is reacted without further purification.

Yield: 171 mg (99% of theory)

### Example 148A

### tert-Butyl {(3S)-4-amino-3-[(tert-butoxycarbonyl)amino]butyl}carbamate

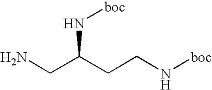

Preparation takes place in analogy to Example 140A from 171 mg (0.52 mmol) of tert-butyl {(3S)-4-azido-3-[(tert-butoxycarbonyl)amino]butyl}carbamate (Example 147A) in 10 ml of ethanol with addition of 20 mg of palladium on activated carbon (10%). The product is reacted without further purification.

Yield: 117 mg (75% of theory)

MS (ESI): m/z=304 (M+H)⁺; 326 (M+Na)⁺

### Example 149A

### (3S)-3-{[(Benzyloxy)carbonyl]amino}6-[(tert-butoxycarbonyl)amino]hexanoyl methylcarbonate

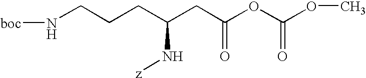

2 g (5.26 mmol) of (3S)-3-{[(benzyloxy)carbonyl]amino}-6-[(tert-butoxycarbonyl)-amino]hexanoic acid and 0.56 g (5.73 mmol) of triethylamine are dissolved in 30 ml of THF under argon and cooled to 0° C. 0.59 g (5.73 mmol) of methyl chloroformate is added, and the mixture is stirred at 0° C. for 3 hours. The reaction mixture is filtered through kieselguhr. The filtrate is reacted directly.

### Example 150A

### Benzyl [(1S)-4-[(tert-butoxycarbonyl)amino]-1-(2-hydroxyethyl)butyl]carbamate

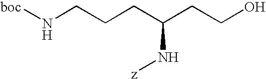

The filtrate of (3S)-3-{[(benzyloxy)carbonyl]amino}-6-[(tert-butoxycarbonyl)-amino]hexanoyl methyl carbonate (Example 149A) is added dropwise to a suspension of 0.49 g (13.14 mmol) of sodium borohydride in 0.6 ml of water at 0° C. The mixture warms slowly to room temperature and is stirred overnight. The reaction solution is concentrated in vacuo, and the residue is mixed with ethyl acetate and water for working up. The organic phase is dried over magnesium sulphate, concentrated in vacuo and dried under high vacuum. The crude product is reacted without further purification.

Yield: 570 mg (30% of theory)

LC-MS (Method 19): Rₜ=2.09 min

MS (EI): m/z=367 (M+H)⁺

### Example 151A

### tert-Butyl [(4S)-4-amino-6-hydroxyhexyl]carbamate

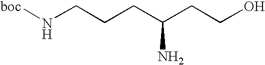

Preparation takes place in analogy to Example 142A from 620 mg (1.69 mmol) of benzyl [(1S)-4-[(tert-butoxycarbonyl)amino]-1-(2-hydroxyethyl)butyl]carbamate (Example 150A) in 60 ml of ethanol with the addition of 100 mg of palladium on activated carbon (10%). The product is reacted without further purification.

Yield: 370 mg (95% of theory)

¹H-NMR (400 MHz, D₂O): δ=1.2-1.6 (m, 6H), 1.4 (s, 9H), 2.6-3.0 (m, 1H), 3.0-3.2 (m, 2H), 3.7-3.9 (m, 2H), 4.6 (br.s, 1H).

### Example 152A

### tert-Butyl {3-[(8S,11S,14S)-14-[(tert-butoxycarbonyl)amino]-8-({[(1R)-4-[(tert-butoxycarbonyl)amino]-1-(hydroxymethyl)butyl]amino}carbonyl)-5,17-dihydroxy-10,13-dioxo-9,12-diazatricyclo[14.3.1.1^{2,6}]henicosa-1(20),2(21),3,5,16,18-hexaen-11-yl]propyl}carbamate

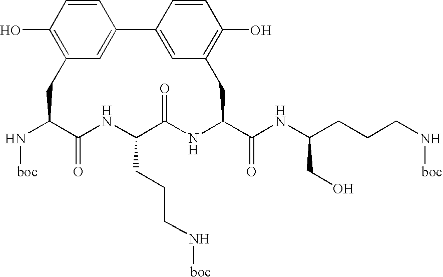

50 mg (0.076 mmol) of the compound from Example 29A and 22 mg (0.10 mmol) of tert-butyl [(4S)4amino-5-hydroxypentyl]carbamate (Example 142A) are dissolved in 1.0 ml of dimethylformamide under argon. Then, at 0° C. (ice bath), 19 mg (0.10 mmol) of EDC and 3.1 mg (0.023 mmol) of HOBt are added. The mixture is slowly warmed to RT and stirred at RT for 12 h. The solution is concentrated in vacuo, and the residue is stirred with water. The remaining solid is filtered off with suction and purified by chromatography on silica gel (mobile phase dichloromethane/isopropanol 30:1 to 10:1).

Yield: 30 mg (47% of theory)

LC-MS (Method 12): Rₜ=2.09 min

MS (EI): m/z=857 (M+H)⁺

### Example 153A

### tert-Butyl {3-[(8S,11S,14S)-8-[({(2S)-2,5-bis[(tert-butoxycarbonyl)amino]pentyl}-amino)carbonyl]-14-[(tert-butoxycarbonyl)amino]-5,17-dihydroxy-10,13-dioxo-9,12-diazatricyclo[14.3.1.1^{2,6}]henicosa-1(20),2(21),3,5,16,18-hexaen-11-yl]propyl}carbamate

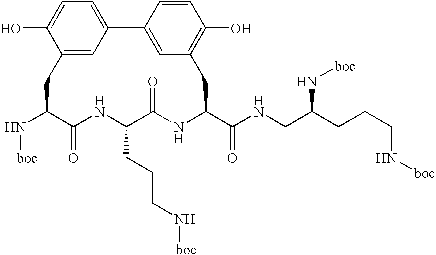

50 mg (0.076 mmol) of the compound from Example 29A and 32 mg (0.10 mmol) of tert-butyl {(4S)-5-amino4-[(tert-butoxycarbonyl)amino]pentyl}carbamate (Example 140A) are dissolved in 1.7 ml of dimethylformamide under argon. Then, at 0° C. (ice bath), 19 mg (0.10 mmol) of EDC and 3.1 mg (0.023 mmol) of HOBt are added. The mixture is warmed slowly to RT and stirred at RT for 12 h. The solution is concentrated in vacuo, and the residue is stirred with water. The remaining solid is filtered off with suction and purified by chromatography on silica gel (mobile phase dichloromethane/isopropanol 30:1 to 10:1).

Yield: 22 mg (30% of theory)

HPLC (Method 12): Rₜ=2.36 min

MS (EI): m/z=956 (M+H)⁺

### Example 154A

### (8S,11S,14S)-14-Amino-11-(3-aminopropyl)-9-ethyl-5,17-dihydroxy-10,13-dioxo-9,12-diazatricyclo[14.3.1.1^{2,6}]henicosa-1(20),2(21),3,5,16,18-hexaene-8-carboxylic acid dihydrochloride

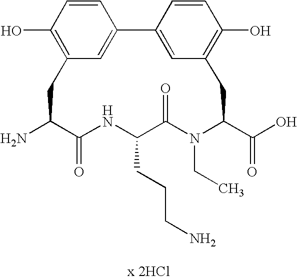

930 mg (0.91 mmol) of the compound from Example 92A are suspended in 260 ml of glacial acetic acid/water/ethanol (4/1/1), 270 mg of palladium on activated carbon (10%) are added, and the mixture is hydrogenated under atmospheric pressure at room temperature for 24 h. Removal of the catalyst by filtration through kieselguhr is followed by evaporation of the filtrate to dryness in vacuo and addition, while stirring, of 36.5 ml of 0.1N hydrochloric acid. The mixture is evaporated to dryness in vacuo and dried to constant weight.

Yield: 500 mg (98% of theory)

LC-MS (Method 20): Rₜ=2.45 min

MS (ESI): m/z=485 (M-2HCl+H)⁺

### Example 155A

### (8S,11S,14S)-14-[(tert-Butoxycarbonyl)amino]-11-{3-[(tert-butoxycarbonyl)-amino]propyl}-9-ethyl-5,17-dihydroxy-10,13-dioxo-9,12-diazatricyclo[14.3.1.1^{2,6}]-henicosa-1(20),2(21),3,5,16,18-hexaene-8-carboxylic acid

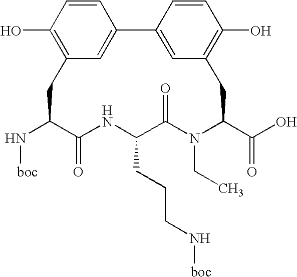

710 mg (1.27 mmol) of the compound from Example 154A are dissolved in 15 ml of water and 6.5 ml (6.5 mmol) of 1N sodium hydroxide solution and, while stirring at room temperature, 834 mg (3.82 mmol) of di-tert-butyl dicarbonate, dissolved in 5.5 ml of methanol, are added. The reaction is complete after one hour (checked by analytical RP-HPLC, mobile phase: acetonitrile/water). The pH is adjusted to 3 by dropwise addition of 0.1N hydrochloric acid. Three extractions each with 20 ml of ethyl acetate are followed by drying with sodium sulphate and evaporation to constant weight in vacuo.

Yield: 770 mg (88% of theory)

HPLC (Method 19): Rₜ=2.16 min

MS (ESI): m/z=685 (M+H)⁺

Examples 156A to 162A listed in the following table are prepared in analogy to the method detailed above for Example 137A from the appropriate starting compounds:

| Ex. | | | |
|---|---|---|---|
| No. | Structure | Prepared from | Analytical data |
| 156A | 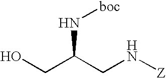 | 3-{[(benzyloxy)- carbonyl]amino}-N- (tert-butoxy- carbonyl)-L-alanine | LC-MS (Method 12): Rₜ =1.79 min. MS (EI): m/z = 325 (M + H)⁺ |
| 157A | 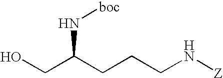 | N⁶-[(benzyloxy)- carbonyl]-N²-(tert- butoxycarbonyl)-L- ornithine | LC-MS (Method 12): Rₜ =1.84 min. MS (EI): m/z = 353 (M + H)⁺ |
| 158A | 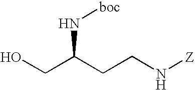 | (2S)4- {[(benzyloxy)- carbonyl]amino}-2- [(tert- butoxycarbonyl)- amino]butane- carboxylic acid | LC-MS (Method 12): Rₜ =1.82 min. MS (EI): m/z = 339 (M + H)⁺ |
| 159A | 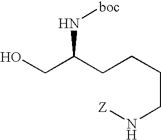 | N⁶-[(benzyloxy)- carbonyl]-N²-(tert- butoxycarbonyl)-L- lysine | LC-MS (Method 12): Rₜ =1.94 min. MS (EI): m/z = 367 (M + H)⁺ |
| 160A | 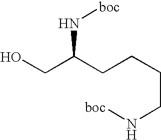 | N²,N⁶-Bis(tert- butoxycarbonyl)-L- lysine | MS (ESI): m/z = 333 (M + H)⁺ |
| 163A | 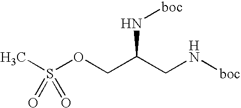 | Example 161A | MS (ESI): m/z = 369 (M + H)⁺ |
| 164A | 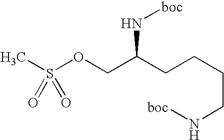 | Example 160A | MS (ESI): m/z = 428 (M + NH₄)⁺ |

Examples 165A and 166A listed in the following table are prepared in analogy to the method detailed above for Example 139A from the appropriate starting compounds:

| Ex. | | | |
|---|---|---|---|
| No. | Structure | Prepared from | Analytical data |
| 165A | 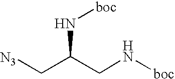 | Example 163A | MS (ESI): m/z = 338 (M + Na)⁺ |
| 166A | 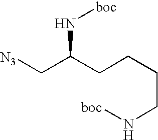 | Example 164A | MS (ESI): m/z = 358 (M + H)⁺ |

Examples 167A and 168A listed in the following table are prepared in analogy to the method detailed above for Example 140A from the appropriate starting compounds:

| Bsp.- | | | |
|---|---|---|---|
| Nr. | Struktur | Hergestellt aus | Analytische Daten |
| 167A | 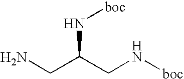 | Beispiel 165A | MS (ESI): m/z = 290 (M + H)⁺ |
| 168A | 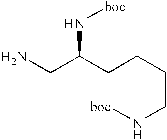 | Beispiel 166A | MS (ESI): m/z = 332 (M + H)⁺ |

Examples 169A to 173A listed in the following table are prepared in analogy to the method detailed above for Example 142A from the appropriate starting compounds:

| Ex. | | | |
|---|---|---|---|
| No. | Structure | Prepared from | Analytical data |
| 169A | 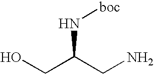 | Example 156A | MS (DCI): m/z = 191 (M + H)⁺ |
| 170A | 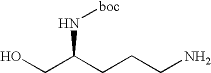 | Example 157A | MS (DCI): m/z = 219 (M + H)⁺ |
| 171A | 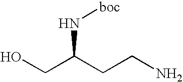 | Example 158A | MS (DCI): m/z = 205 (M + H)⁺ |
| 172A | 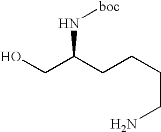 | Example 159A | MS (ESI): m/z = 233 (M + H)⁺ |
| 173A | 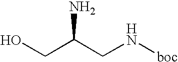 | Example 162A | MS (DCI): m/z = 191 (M + H)⁺ |

Examples 174A to 185A listed in the following table are prepared in analogy to the method of Example 152A from the appropriate starting compounds:

| | Precursor | | |
|---|---|---|---|
| Example | of | | |
| No. | example | Structure | Analytical data |
| 174A | 29A and 169A | 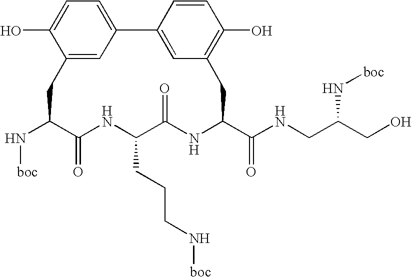 | LC-MS (Method. 17): Rₜ =2.22 min. MS (EI): m/z = 829 (M + H)⁺. |
| 175A | 29A and 170A | 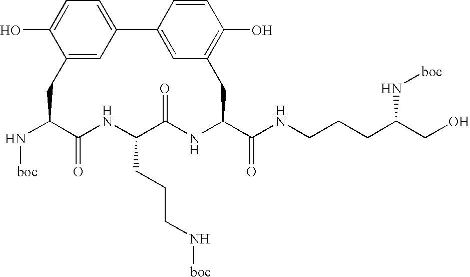 | LC-MS (Method 17): Rₜ = 2.21 min. MS (EI): m/z = 857 (M + H)⁺. |
| 176A | 29A and 171A | 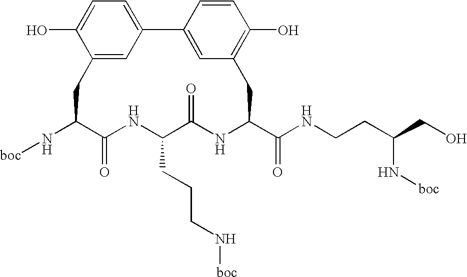 | LC-MS (Method 12): Rₜ = 1.99 min. MS (EI): m/z = 843 (M + H)⁺. |
| 177A | 29A and 172A | 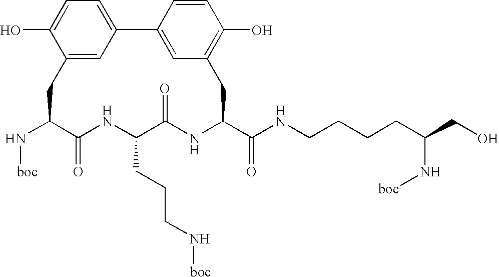 | LC-MS (Method 17): Rₜ = 2.25 min. MS (EI): m/z = 871 (M + H)⁺. |
| 178A | 29A and 144A | 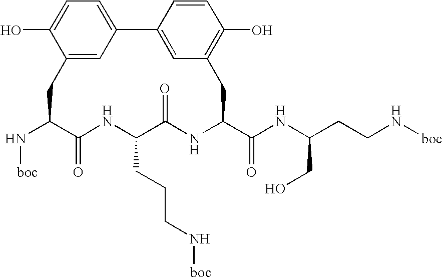 | LC-MS (Method 12): Rₜ = 2.04 min. MS (EI): m/z = 843 (M + H)⁺. |
| 179A | 29A and 173A | 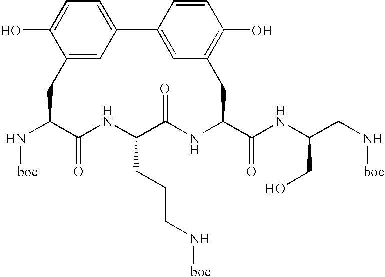 | LC-MS (Method 12): Rₜ = 2.03 min. MS (EI): m/z = 829 (M + H)⁺. |
| 180A | 29A and 148A | 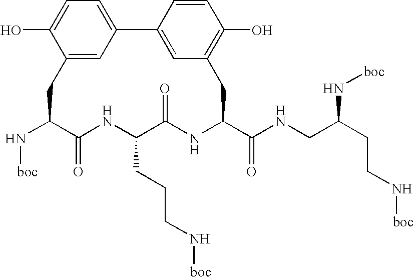 | LC-MS (Method 19): Rₜ = 2.50 min. MS (EI): m/z = 942 (M + H)⁺. |
| 181A | 29A and 167A | 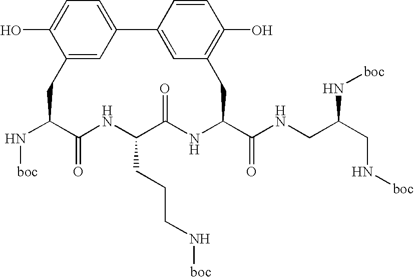 | LC-MS (Method 12): Rₜ = 2.37 min. MS (EI): m/z = 928 (M + H)⁺. |
| 182A | 29A and 168A | 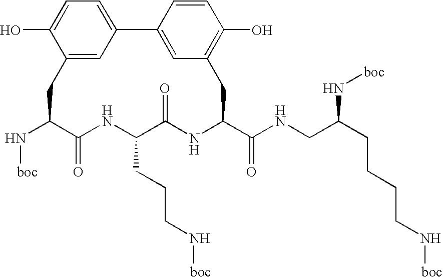 | LC-MS (Method 17): Rₜ = 2.59 min. MS (EI): m/z = 970 (M + H)⁺. |
| 183A | 155A and 171A | 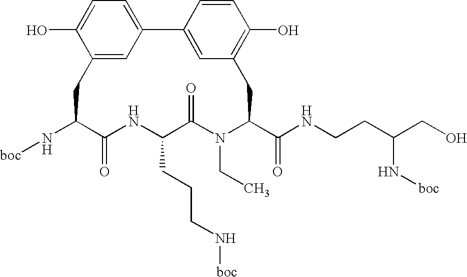 | LC-MS (Methode 12): Rₜ = 2.09 min. MS (EI): m/z = 871 (M + H)⁺. |
| 184A | 29A and 151A | 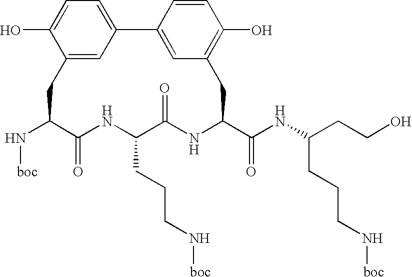 | LC-MS (Method 17): Rₜ = 2.25 min. MS (EI): m/z = 871 (M + H)⁺ |
| 185A | 29A and 191A | 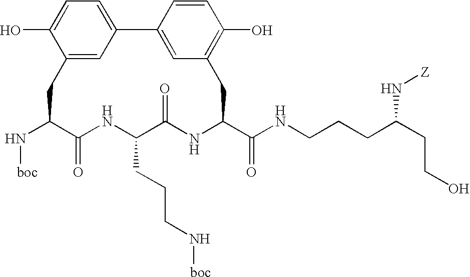 | LC-MS (Method 19): Rₜ = 2.24 min. MS (EI): m/z = 905 (M + H)⁺ |

Example 186A detailed in the following table is prepared in analogy to the method of Example 120A from the appropriate starting compounds:

| | Precursor | | |
|---|---|---|---|
| Example | of | | |
| No. | example | Structure | Analytical data |
| 186A | 185A | 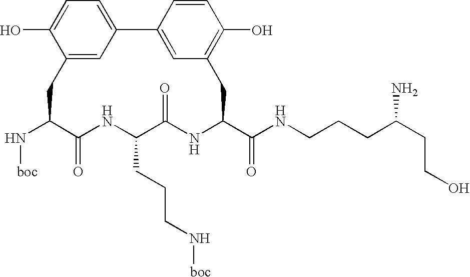 | LC-MS (Method 19): Rₜ = 1.71 min. MS (EI): m/z = 771 (M + H)⁺ |

Examples 187A to 191A listed in the following table are prepared in analogy to the stated method from the appropriate starting compounds:

| | Prepared in | | |
|---|---|---|---|
| Example | analogy to | | |
| No. | example | Structure | Analytical data |
| 187A | 137A | 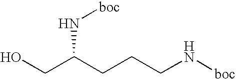 | MS (ESI): m/z = 319 (M + H)⁺. |
| 188A | 138A from example 187A | 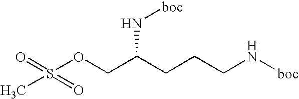 | MS (DCI): m/z = 414 (M + NH₄)⁺. |
| 189A | 139A from example 188A | 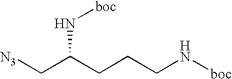 | MS (DCI): m/z = 361 (M + NH₄)⁺. |
| 190A | 140A from example 189A | 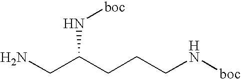 | MS (ESI): m/z = 318 (M + H)⁺. |
| 191A | 15A from example 150A | 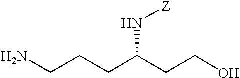 | LC-MS (Method 19): Rₜ = 1.06 min. MS (EI): m/z = 267 (M-HCl + H)⁺ |

Example 1 92A detailed in the following table is prepared in analogy to the method of Example 152A from the appropriate starting compounds:

| | Precursor | | |
|---|---|---|---|
| Example | of | | |
| No. | example | Structure | Analytical data |
| 192A | 29A and 190A | 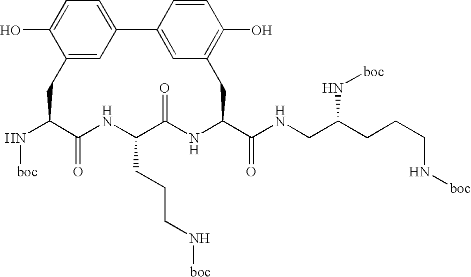 | LC-MS (Method 17): Rₜ = 2.58 min. MS (EI): m/z = 956 (M + H)⁺ |

### Exemplary Embodiments

Exemplary embodiments can be synthesized starting from partially protected biphenomycin derivatives (such as, for example, 29A).

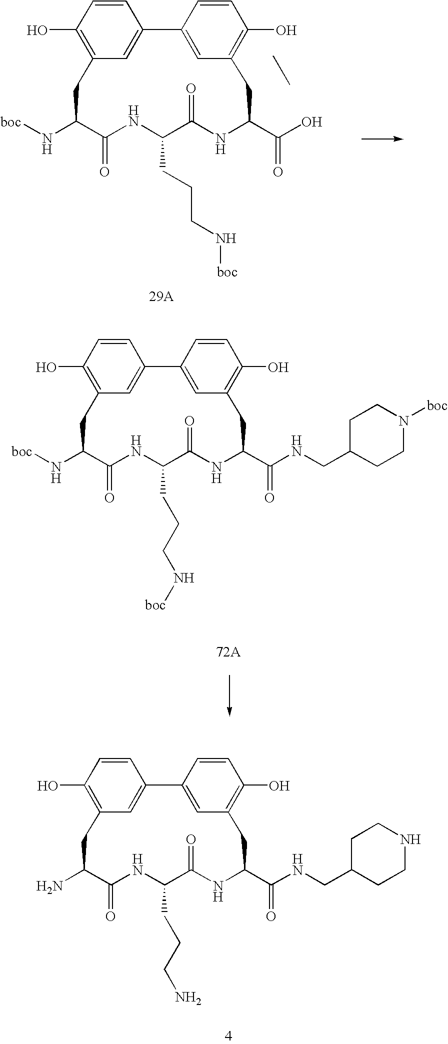

### Example 1

### (8S,11S,14S)-14-Amino-N-(2-aminoethyl)-11-[(2R)-3-amino-2-hydroxypropyl]-5,17-dihydroxy-9-methyl-10,13-dioxo-9,12-diazatricyclo[14.3.1.1^{2,6}]henicosa-1(20),2(21),3,5,16,18-hexaene-8-carboxamide trihydrochloride

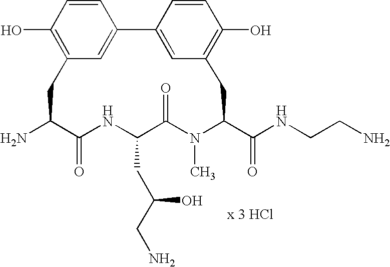

1 ml of 4N hydrogen chloride/dioxane solution is cooled in an ice bath and, while stirring, 15 mg (0.02 mmol) of the compound from Example 68A are added. After a short time, the ice bath is removed, and the mixture is stirred at RT for one hour. The product is obtained by evaporation to dryness in vacuo.

Yield: 11 mg (94% of theory)

LC-MS (Method 17): Rₜ=0.24 min

MS (EI): m/z=528 (M-3HCl+H)⁺

¹H-NMR (400 MHz, D₂O): δ=1.97 (m_{c}, 2H), 2.85 (m_{c}, 1H), 2.90 (s, 3H), 2.96-3.32 (m, 6H), 3.41-3.60 (m, 3H), 3.65 (m, 1H), 3.73 (m_{c}, 1H), 3.94 (m_{c}, 1H), 4.42 (m_{c}, 1H), 5.08 (m_{c}, 1H), 5.62 (m_{c}, 1H), 6.88 (m_{c}, 2H), 6.95 (s, 1H), 7.04 (s, 1H), 7.40 (m_{c}, 1H), 7.48 (m _{c}, 1H).

### Example 2

### (8S,11S,14S)-14-Amino-11-[(2R)-3-amino-2-hydroxypropyl]-5,17-dihydroxy-9-methyl-10,13-dioxo-N-piperidin-4-yl-9,12-diazatricyclo[14.3.1.1^{2,6}]henicosa-1(20),2(21),3,5,16,18-hexaene-8-carboxamide trihydrochloride

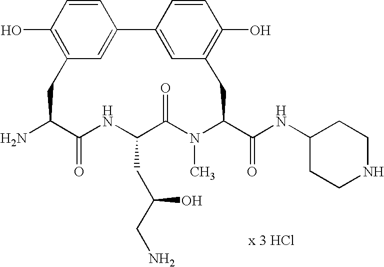

Preparation takes place in analogy to Example 1 from 14 mg (0.02 mmol) of the compound from Example69A with 1 ml of 4N hydrogen chloride/dioxane solution.

Yield: 10 mg (92% of theory)

LC-MS (Method 17): Rₜ=0.28 min

MS (EI): m/z=568 (M-3HCl+H)⁺

### Example 3

### (8S,11S,14S)-14-Amino-N-(2-aminoethyl)-11-(3-aminopropyl)-5,17-dihydroxy-9-methyl-10,13-dioxo-9,12-diazatricyclo[14.3.1.1^{2,6}]henicosa-1(20),2(21),3,5,16,18-hexaene-8-carboxamide trihydrochloride

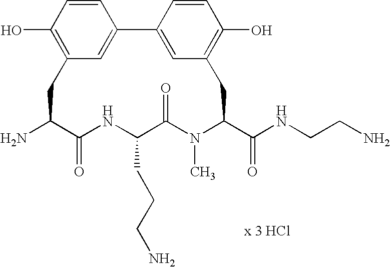

1 ml of ice-cold 4N hydrogen chloride/dioxane solution is poured over 4 mg (0.005 mmol) of the compound from Example 70A and stirred for one hour, during which the temperature rises to RT. The mixture is evaporated to dryness in vacuo until the weight is constant.

Yield: 3 mg (98% of theory)

LC-MS (Method 17): Rₜ=0.28 min

MS (EI): m/z=512 (M-3HCl+H)⁺

### Example 4

### (8S,11S,14S)-14-Amino-11-(3-aminopropyl)-5,17-dihydroxy-10,13-dioxo-N-(piperidin-4-ylmethyl)-9,12-diazatricyclo[14.3.1.1^{2,6}]henicosa-1(20),2(21),3,5,16,18-hexaene-8-carboxamide trihydrochloride

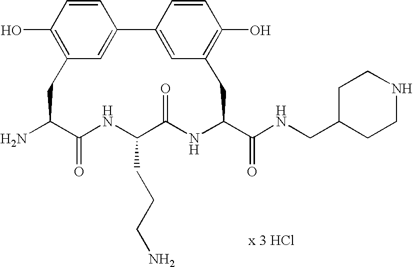

Preparation takes place in analogy to Example 1 from 8 mg (0.01 mmol) of the compound from Example 72A with 1 ml of 4N hydrogen chloride/dioxane solution.

Yield: 5 mg (73% of theory)

LC-MS (Method 12): Rₜ=0.22 min

MS (EI): m/z=838 (M-3HCl+H)⁺

### Example 5

### (8S,11S,14S)-14-Amino-11-(3-aminopropyl)-5,17-dihydroxy-10,13-dioxo-N-piperidin-4-yl-9,12-diazatricyclo[14.3.1.1^{2,6}]henicosa-1(20),2(21),3,5,16,18-hexaene-8-carboxamide trihydrochloride

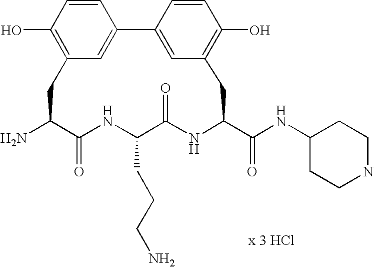

Preparation takes place in analogy to Example 1 from 11 mg (0.01 mmol) of the compound from Example 73A with 1 ml of 4N hydrogen chloride/dioxane solution.

Yield: 8.4 mg (99% of theory)

LC-MS (Method 19): Rₜ=0.24 min

MS (EI): m/z=538 (M-3HCl+H)⁺

### Example 6

### (8S,11S,14S)-14-Amino-N-(2-aminoethyl)-11-(3-aminopropyl)-5,17-dihydroxy-10,13-dioxo-9,12-diazatricyclo[14.3.1.1^{2,6}]henicosa-1 (20),2(21),3,5,16,18-hexaene-8-carboxamide trihydrochloride

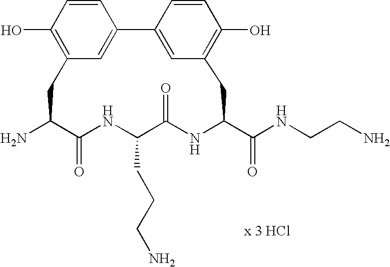

36.8 mg (0.05 mmol) of the compound from Example 74A are added to 2 ml of an ice-cold 4N hydrogen chloride/dioxane solution and stirred at RT for one hour. The mixture is then evaporated to dryness in vacuo and dried in a desiccator (diphosphorus pentoxide) to constant weight.

Yield: 29 mg (98% of theory)

LC-MS (Method 20): Rₜ=2.01 min

MS (EI): m/z=499 (M-3HCl+H)⁺

¹H-NMR (400 MHz, D₂O): δ=1.52-1.92 (m, 4H), 2.85 (m_{c}, 1H), 2.93 (m_{c}, 2H), 3.03 (m_{c}, 1H), 3.11 (m_{c}, 2H, 3.23 (m_{c}, 1H), 3.42-3.62 (m, 3H), 4.42 (m_{c}, 1H), 4.7 (m, 1H, under D₂O), 4.77 (m_{c}, 1H), 6.88 (m_{c}, 2H), 6.97 (s, 1H), 7.23. (s, 1H), 7.34 (m_{c}, 1H), 7.42 (M_{c}, 1H).

### Example 7

### (8S,11S,14S)-14-Amino-N-(3-amino-2-hydroxypropyl)-11-(3-aminopropyl)-5,17-dihydroxy-10,13-dioxo-9,12-diazatricyclo[14.3.1.1^{2,6}]henicosa-1(20),2(21),3,5,16,18-hexaene-8-carboxamide trihydrochloride

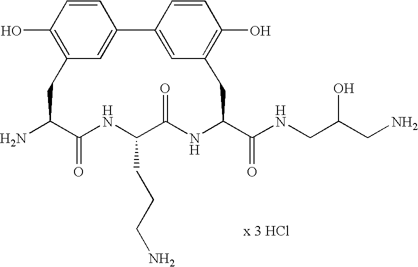

Preparation takes place in analogy to Example 1 from 10 mg (0.01 mmol) of the compound from Example 75A with 1.05 ml of 4N hydrogen chloride/dioxane solution.

Yield: 8 mg (quantitative)

LC-MS (Method 12): Rₜ=0.23 min

MS (EI): m/z=528 (M-3HCl+H)⁺

### Example 8

### (8S,11S,14S)-14-Amino-N,11-bis(3-aminopropyl)-5,17-dihydroxy-10,13-dioxo-9,12-diazatricyclo[14.3.1.1^{2,6}]henicosa-1(20),2(21),3,5,16,18-hexaene-8-carboxamide trihydrochloride

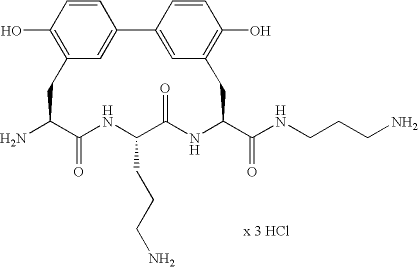

Preparation takes place in analogy to Example 1 from 7.5 mg (0.01 mmol) of the compound from Example 76A with 1 ml of 4N hydrogen chloride/dioxane solution.

Yield: 5.8 mg (quantitative)

LC-MS (Method 12): Rₜ=0.22 min

MS (EI): m/z 512 (M-3HCl+H)⁺

### Example 9

### (8S,11S,14S)-14-Amino-11-(3-aminopropyl)-5,17-dihydroxy-N-[2-(methylamino)ethyl]-10,13-dioxo-9,12-diazatricyclo[14.3.1.1^{2,6}]henicosa-1(20),2(21),3,5,16,18-hexaene-8-carboxamide trihydrochloride

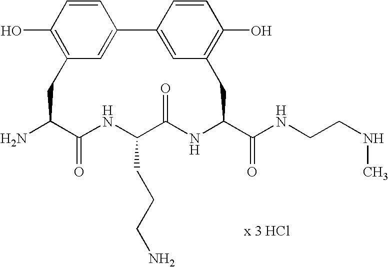

Preparation takes place in analogy to Example 1 from 6 mg (0.01 mmol) of the compound from Example 77A with 1 ml of 4N hydrogen chloride/dioxane solution.

Yield: 5 mg (quantitative)

LC-MS (Method 12): Rₜ=0.22 min

MS (EI): m/z=512 (M-3HCl+H)⁺

### Example 10

### (8S,11S,14S)-14-Amino-11-[(2R)-3-amino-2-hydroxypropyl]-N-(3-amino-2-hydroxypropyl)-5,17-dihydroxy-10,13-dioxo-9,12-diazatricyclo-14.3.1.1^{2,6}]henicosa-1(20),2(21),3,5,16,18-hexaene-8-carboxamide trihydrochloride

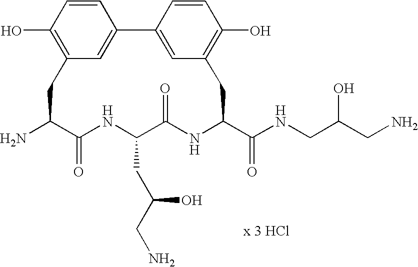

Preparation takes place in analogy to Example 1 from 50 mg (0.057 mmol) of the compound from Example 80A with 1 ml of 4N hydrogen chloride/dioxane solution.

Yield: 38 mg (99% of theory)

LC-MS (Method 17): Rₜ=0.22 min

MS (EI): m/z=545 (M-3HCl+H)⁺

### Example 11

### (8S,11S,14)-14-Amino-N-(2-aminoethyl)-11-[(2R)-3-amino-2-hydroxypropyl]-5,17-dihydroxy-10,13-dioxo-9,12-diazatricyclo[14.3.1.1^{2,6}]henicosa-1(20),2(21),3,5,16,18-hexaene-8-carboxamide trihydrochloride

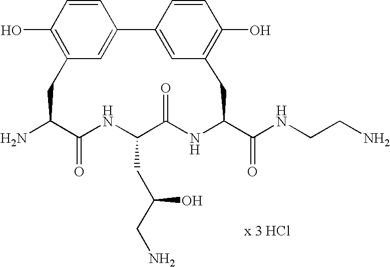

4.1 mg (0.005 mmol) of the compound from Example 81A are put into 2 ml of an ice-cold 4N hydrogen chloride/dioxane solution and stirred at RT for one hour. Evaporation of the solvent in vacuo and drying in a desiccator (diphosphorus pentoxide) results in a colourless residue.

Yield: 3.5 mg (99% of theory)

LC-MS (Method 12): Rₜ=0.22 min

MS (EI): m/z=515 (M-3HCl+H)⁺

### Example 12

### (8S,11S,14S)-14-Amino-11-(3-aminopropyl)-5,17-dihydroxy-9-methyl-10,13-dioxo-N-(2-piperidin-2-ylethyl)-9,12-diazatricyclo[14.3.1.1^{2,6}]henicosa-1(20),2(21),3,5,16,18-hexaene-8-carboxamide trihydrobromide

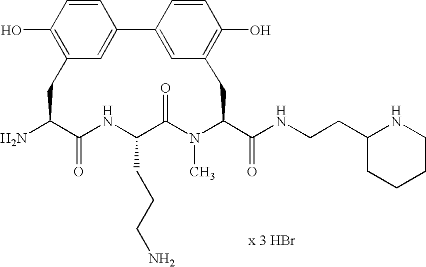

46 mg (0.04 mmol) of the compound from Example 82A are dissolved in 1 ml of 33% strength hydrobromic acid solution in acetic acid and stirred at RT for 45 min. The solvent is then removed in vacuo, and the crude product is stirred in methanol and the solvent is again removed in vacuo.

Yield: 33 mg (98% of theory)

LC-MS (Method 12): Rₜ=0.27 min

MS (EI): m/z=581 (M-3HBr+H)⁺

¹H-NMR (400 MHz, D₂O): δ=1.05-1.95 (m, 12H), 2.75-3.45 (m, 13H), 3.55 (m_{c}, 1H), 4.45 (m_{c}, 1H), 4.92 (m_{c}, 1H), 5.60 (m_{c}, 1H), 6.88 (m_{c}, 2H), 6.95 (m_{c}, 1H), 7.04 (m_{c}, 1H), 7.38 (m_{c}, 1H), 7.48 (m_{c}, 1H).

### Example 13

### (8S,11S,14S)-14-Amino-11-(3-aminopropyl)-5,17-dihydroxy-9-methyl-10,13-dioxo-N-(piperidin-2-ylmethyl)-9,12-diazatricyclo[14.3.1.1^{2,6}]henicosa-1(20),2(21),3,5,16,18-hexaene-8-carboxamide trihydrobromide

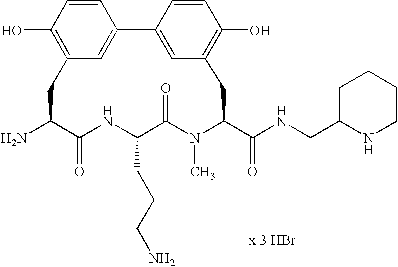

Preparation takes place in analogy to Example 14 from 48 mg (0.043 mmol) of the compound from Example 83A with 1 ml of 33% strength hydrobromic acid solution in acetic acid.

Yield: 30 mg (86% of theory)

LC-MS (Method 12): Rₜ=0.27 min

MS (EI): m/z=567 (M-3HBr+H)⁺

¹H-NMR (400 MHz, D₂O): δ=1.35-1.95 (m, 10H), 2.86 (s, 3H), 2.8-3.5 (m, 10H), 3.55 (m_{c}, 1H), 4.47 (m_{c}, 1H), 4.90 (m_{c}, 1H), 5.65 (m_{c}, 1H), 6.88 (m_{c}, 2H), 6.94 (s, 1H), 7.04 (s, 1H), 7.40 (m_{c}, 1H), 7.48 (m_{c}, 1H).

### Example 14

### (8S,11S,14S)-14-Amino-N-(2-aminoethyl)-11-(3-aminopropyl)-5,17-dihydroxy-10,13-dioxo-9,12-diazatricyclo[14.3.1.1^{2,6}]henicosa-1(20),2(21),3,5,16,18-hexaene-8-carboxamide

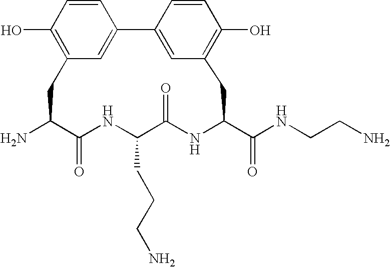

55 mg (0.09 mmol) of the compound from Example 6 are dissolved in 1 ml of water and 0.1 ml of diethylamine and converted into the free base by preparative HPLC (Method 21).

Yield: 28 mg (62% of theory)

LC-MS (Method 20): Rₜ=2.01 min

MS (EI): m/z=499 (M+H)⁺

NMR (400 MHz, D₂O): δ=1.52-1.92 (m, 4H), 2.85 (m_{c}, 1H), 2.93 (m_{c}, 2H), 3.03 (m_{c}, 1H), 3.11 (m_{c}, 2H), 3.23 (m_{c}, 1H), 3.42-3.62 (m, 3H), 4.42 (m_{c}, 1H), 4.7 (m, 1H), 4.77 (m_{c}, 1H), 6.88 (m_{c}, 2H), 6.97 (s, 1H), 7.23 (s, 1H), 7.34 (m_{c}, 1H), 7.42 (m_{c}, 1H).

### Example 15

### (8S,11S,14S)-14-Amino-N-(2-aminoethyl)-11-(3-aminopropyl)-5,17-dihydroxy-10,13-dioxo-9,12-diazatricyclo[14.3.1.1^{2,6}]henicosa-1 (20),2(21),3,5,16,18-hexaene-8-carboxamide tris(trifluoroacetate)

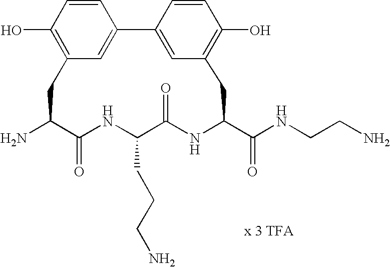

14 mg (0.03 mmol) of the compound from Example 14 are dissolved in 0.5 ml of dioxane, mixed with 11 μl (0.14 mmol) of trifluoroacetic acid and stirred at RT for 20 min. The solvent is then removed in vacuo, and the crude product is stirred in dioxane and the solvent is again removed in vacuo.

Yield: 18 mg (62% of theory)

LC-MS (Method 20): Rₜ=1.89 min

MS (EI): m/z 499 (M-3TFA+H)⁺

### Example 16

### (8S,11S,14S)-14-Amino-N,11-bis(2-aminoethyl)-5,17-dihydroxy-9-methyl-10,13-dioxo-9,12-diazatricyclo[14.3.1.1^{2,6}]henicosa-1(20),2(21),3,5,16,18-hexaene-8-carboxamide tris(trifluoroacetate)

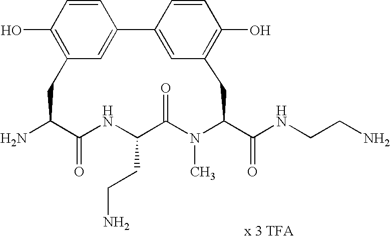

Preparation takes place in analogy to Example 3 from 28 mg (0.047 mmol) of the compound from Example 108A with 2 ml of 4N dioxane/hydrogen chloride solution. The crude product is purified by HPLC (Kromasil 100C18, mobile phase acetonitrile/0.2% aqueous trifluoroacetic acid 1:3).

Yield: 12 mg (30% of theory)

LC-MS (Method 20): Rₜ=1.92 min

MS (EI): m/z=499 (M-3TFA+H)⁺

¹H-NMR (400 MHz, D₂O): δ=2.13 (m_{c}, 1H), 2.27 (m_{c}, 1H), 3.01 (s, 3H), 3.1-3.33 (m, 6H), 3.43 (m_{c}, 1H), 3.6-3.75 (m, 3H), 4.58 (m_{c}, 1H), 5.13 (m_{c}, 1H), 5.78 (m_{c}, 1H), 7.03 (m_{c}, 2H), 7.08 (s, 1H), 7.16 (s, 1H), 7.55 (d, 1H), 7.63 (d, 1H).

Examples 17 to 28 listed in the following table are prepared in analogy to the methods detailed above from the appropriate starting compounds:

| | | Prepared in | |
|---|---|---|---|
| Example | | analogy to | |
| No. | Structure | example | Analytical data |
| 17 | 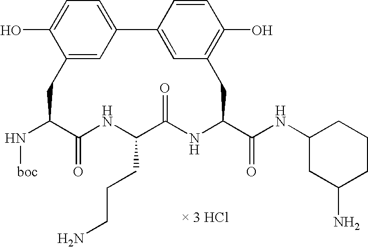 | 3 from example 116A | LC-MS (Method 20): Rₜ = 2.01 min. MS (EI): m/z = 553 (M-3HCl + H)⁺ |
| 18 | 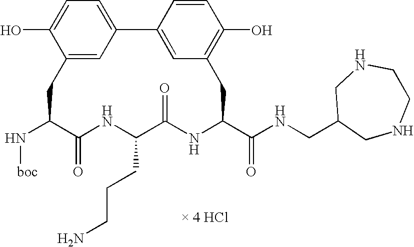 | 3 from example 120A | LC-MS (Method 20): Rₜ = 1.02 min. MS (EI): m/z = 554 (M-4HCl + H)⁺ |
| 19 | 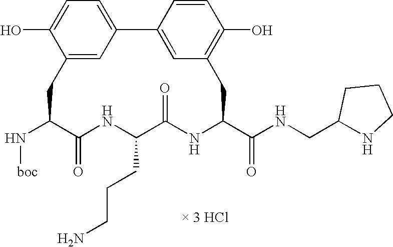 | 3 from example 118A | LC-MS (Method 20): Rₜ = 2.20 min. MS (EI): m/z = 539 (M-3HCl + H)⁺ |
| 20 | 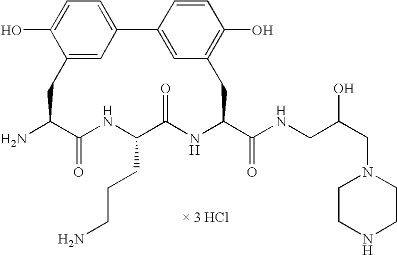 | 3 from example 119A | LC-MS (Method 20): Rₜ = 2.03 min. MS (EI): m/z = 598 (M-3HCl + H)⁺ |
| 21 | 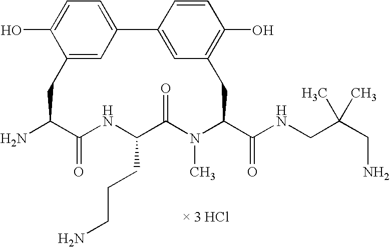 | 3 from example 112A | LC-MS (Method 20): Rₜ = 2.11 min. MS (EI): m/z = 555 (M-3HCl + H)⁺ |
| 22 | 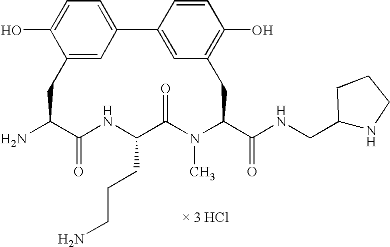 | 3 from example 113A | LC-MS (Method 20): Rₜ = 2.11 min. MS (EI): m/z = 553 (M-3HCl + H)⁺ |
| 23 | 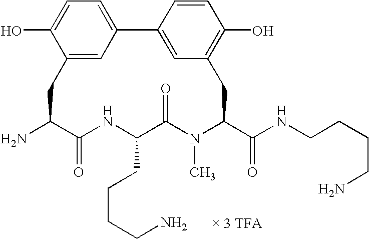 | 16 from example 110A | LC-MS (Method 20): Rₜ = 2.19 min. MS (EI): m/z = 555 (M-3TFA + H)⁺ |
| | | | ¹H-NMR (400 MHz, D₂O): δ = 1.4 |
| | | | (m_{c}, 2H), 1.5-1.7 (m_{c}, 7H), 1.78 (m_{c}, |
| | | | 1H), 2.7-3.35 (m, 13H), 3.55 (m_{c}, |
| | | | 1H), 4.43 (m_{c}, 1H), 4.88 (m_{c}, 1H), |
| | | | 5.6 (m_{c}, 1H), 6.88 (m_{c}, 2H), 6.97 (s, |
| | | | 1H), 7.05 (s, 1H), 7.41 (d, 1H), 7.48 |
| | | | (d, 1H). |
| 24 | 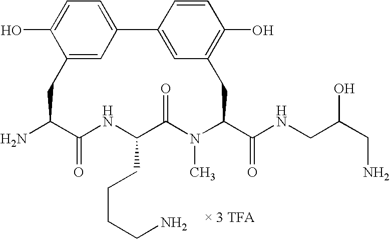 | 16 from example 111A | LC-MS (Method 20): Rₜ = 2.18 min. MS (EI): m/z = 557 (M-3TFA + H)⁺ |
| | | | ¹H-NMR (400 MHz, D₂O): δ = 1.4 |
| | | | (m_{c}, 2H), 1.5-1.7 (m_{c}, 3H), 1.78 (m_{c}, |
| | | | 1H), 2.75-3.45 (m, 12H), 3.55 (m_{c}, |
| | | | 1H), 3.95 (m_{c}, 1H), 4.43 (m_{c}, 1H), |
| | | | 4.88 (m_{c}, 1H), 5.65 (m_{c}, 1H), 6.88 |
| | | | (m_{c}, 2H), 6.96 (s, 1H), 7.05 (s, 1H), |
| | | | 7.42 (d, 1H), 7.48 (d, 1H). |
| 25 | 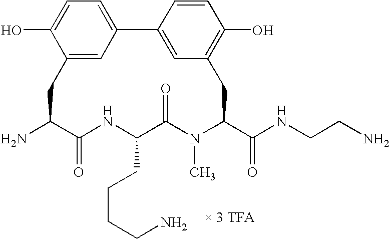 | 16 from example 109A | LC-MS (Method 20): Rₜ = 2.13 min. MS (EI): m/z = 527 (M-3TFA + H)⁺ |
| | | | ¹H-NMR (400 MHz, D₂O): δ = 1.27 |
| | | | (m_{c}, 2H), 1.4-1.6 (m_{c}, 3H), 1.7 (m_{c}, |
| | | | 1H), 2.7-3.05 (m, 9H), 3.17 (m_{c}, |
| | | | 1H), 3.3-3.5 (m, 3H), 4.30 (m_{c}, 1H), |
| | | | 4.75 (m_{c}, 1H), 5.52 (m_{c}, 1H), 6.67 |
| | | | (m_{c}, 2H), 6.83 (s, 1H), 6.92 (s, 1H), |
| | | | 7.28 (d, 1H), 7.37 (d, 1H). |
| 26 | 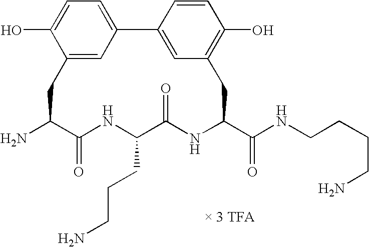 | 16 from example 114A | LC-MS (Method 20): Rₜ = 2.09 min. MS (EI): m/z = 527 (M-3TFA + H)⁺ |
| | | | ¹H-NMR (400 MHz, D₂O): δ = 1.45- |
| | | | 1.85 (m_{c}, 8H), 2.8-3.07 (m, 6H), |
| | | | 3.15 (m_{c}, 2H), 3.28 (m_{c}, 1H), 3.55 |
| | | | (m_{c}, 1H), 4.42 (m_{c}, 1H), 4.7-4.8 (m, |
| | | | 2H under D₂O signal), 6.89 (m_{c}, |
| | | | 2H), 6.96 (s, 1H), 7.25 (s, 1H), 7.35 |
| | | | (d, 1H), 7.43 (d, 1H). |
| 27 | 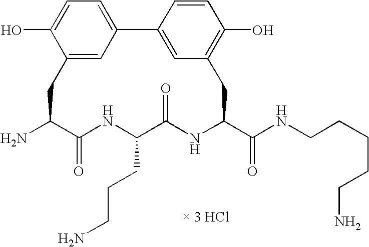 | 3 from example 115A | LC-MS (Method 20): Rₜ = 2.16 min. MS (EI): m/z = 541 (M-3HCl + H)⁺ |
| 28 | 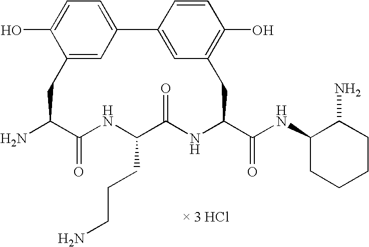 | 3 from example 121A | LC-MS (Method 20): Rₜ = 2.19 min. MS (EI): m/z = 553 (M-3HCl + H)⁺ |

### Example 29

### (8S, 11S, 14S)-14-Amino-N,11-bis(3-aminopropyl)-5,17-dihydroxy-10,13-dioxo-9,12-diazatricyclo[14.3.1.1^{2,6}]henicosa-1(20),2(21),3,5,16,18-hexaene-8-carboxamide tris(trifluoroacetate)

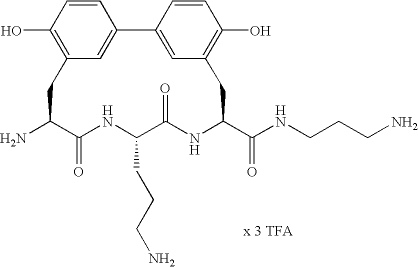

16.3 mg (0.03 mmol) of the compound from Example 8 are converted into tris(trifluoroacetate) by preparative HPLC (Kromasil 100C18, mobile phase acetonitrile/0.2% aqueous trifluoroacetic acid 1:3).

Yield: 10.4 mg (45% of theory)

LC-MS (Method 20): Rₜ=1.93 min

MS (EI): m/z=513 (M-3TFA+H)⁺

¹H-NMR (400 MHz, D₂O): δ=1.5-1.9 (m_{c}, 6H), 2.7-3.1 (m, 6H), 3.15-3.26 (m, 2H), 3.35 (m_{c}, 1H), 3.55 (m_{c}, 1H), 4.42 (m_{c}, 1H), 4.7-4.8 (m, 2H under D₂O signal), 6.89 (m_{c}, 2H), 6.96 (s, 1H), 7.25 (s, 1H), 7.35 (d, 1H), 7.43 (d, 1H).

Examples 30 and 31 listed in the following table are prepared in analogy to the methods detailed above from the appropriate starting compounds:

| | | Prepared in | |
|---|---|---|---|
| Example | | analogy to | |
| No. | Structure | example | Analytical data |
| 30 | 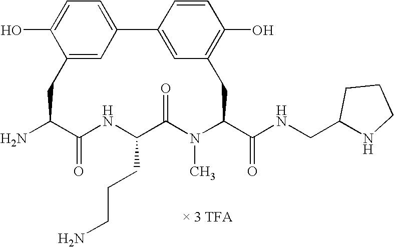 | 29 from example 22 | LC-MS (Method 20): Rₜ = 2.25 min. MS (EI): m/z = 553 (M-3TFA + H)⁺ |
| | | | ¹H-NMR (400 MHz, D₂O): δ = 1.6- |
| | | | 1.75 (m, 4H), 1.8-2.1 (m, 3H), 2.15 |
| | | | (m_{c}, 1H), 2.88 (s, 3H), 2.95 (m_{c}, |
| | | | 2H), 3.07 (m_{c}, 2H), 3.2-3.35 (m, |
| | | | 3H), 3.43-3.6 (m, 3H), 3.72 (m_{c}, |
| | | | 1H), 4.45 (m_{c}, 1H), 4.92 (m_{c}, 1H), |
| | | | 5.65 (m_{c}, 1H), 6.88 (m_{c}, 2H), 6.96 |
| | | | (s, 1H), 7.04 (s, 1H), 7.42 (d, 1H), |
| | | | 7.49 (d, 1H). |
| 31 | 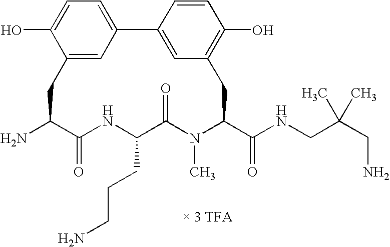 | 29 from example 21 | LC-MS (Method 20): Rₜ = 2.35 min. MS (EI): m/z = 555 (M-3TFA + H)⁺ |
| | | | ¹H-NMR (400 MHz, D₂O): δ = 0.94 |
| | | | (s, 6H), 1.6-1.75 (m, 3H), 1.85 (m_{c}, |
| | | | 1H), 2.75 (m_{c}, 2H), 2.9 (s, 3H), 2.93 |
| | | | (m_{c}, 2H), 3.0-3.17 (m, 3H), 3.2-3.35 |
| | | | (m, 2H), 3.56 (m_{c}, 1H), 4.45 (m_{c}, |
| | | | 1H), 4.92 (m_{c}, 1H), 5.67 (m_{c}, 1H), |
| | | | 6.9 (m_{c}, 2H), 6.96 (s, 1H), 7.05 (s, |
| | | | 1H), 7.42 (d, 1H), 7.49 (d, 1H). |

### Example 32

### (8S,11S,14S)-14-Amino-N-(2-aminoethyl)-11-(3-aminopropyl)-9-ethyl-5,17-dihydroxy-10,13-dioxo-9,12-diazatricyclo[14.3.1.1^{2,6}]henicosa-1(20),2(21),3,5,16,18-hexaene-8-carboxamide tris(trifluoroacetate)

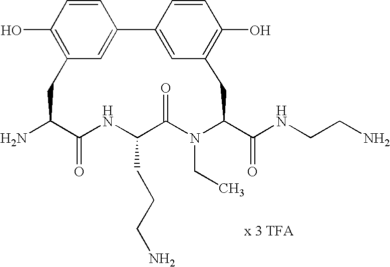

17 mg (0.02 mmol) of the compound from Example 93A are suspended in 5 ml of glacial acetic acid/water/tetrahydrofuran (4:1:1), mixed with 5 mg of palladium on activated carbon (10%) and hydrogenated under atmospheric pressure at RT for 1 day. The catalyst is removed on a membrane filter, and the filtrate is concentrated in vacuo. The crude product is purified by HPLC (Kromasil 100C18, mobile phase acetonitrile/0.2% aqueous trifluoroacetic acid 1:3).

Yield: 6 mg (39% of theory)

LC-MS (Method 20): Rₜ=2.0 min

MS (EI): m/z=527 (M-3TFA+H)⁺

Examples 33 and 34 listed in the following table are prepared in analogy to the methods detailed above from the appropriate starting compounds:

| | | Prepared in | |
|---|---|---|---|
| Example | | analogy to | |
| No. | Structure | example | Analytical data |
| 33 | 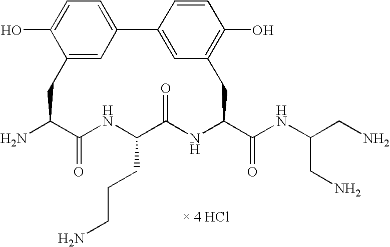 | 3 from example 122A | LC-MS (Method 20): Rₜ = 0.56 min. MS (EI): m/z = 528 (M-4HCl + H)⁺ |
| 34 | 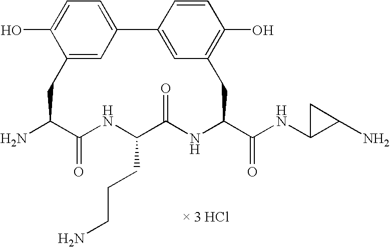 | 3 aus Beispiel 123A | LC-MS (Method 20): Rₜ = 2.14 min. MS (EI): m/z = 511 (M-3HCl + H)⁺ |

Examples 35 to 41 listed in the following table are prepared in analogy to the method of Example 6 or 15 from the appropriate starting compounds:

| | | Precursor | |
|---|---|---|---|
| | | of | |
| Example | | Example | |
| No. | Structure | No | Analytical data |
| 35 | 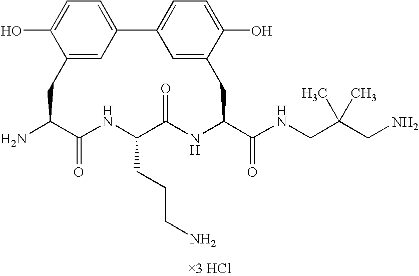 | 129A | LC-MS (Method 20): R₁ = 2.08 min. MS (ESI): m/z = 541 (M − 3HCl + H)⁺. ¹H-NMR (400 MHz, D₂O): δ = 0.94 (s, 6H),1.5-1.85 (m, 4H), 2.74 (s, 2H), 2.8-3.05 (m, 5H), 3.1-3.25 (m, 2H), 3.52 |
| #(m_{c}, 1H), 4.41 (m_{c}, 1H), 4.6 (m, 1H, under D₂O), 4.77 (m_{c}, 1H), 6.83-6.9 (m, 2H), 6.96 (s, 1H), 7.23 (s, 1H), 7.33 (d, 1H), 7.4 (d, 1H). | | | |
| 36 | 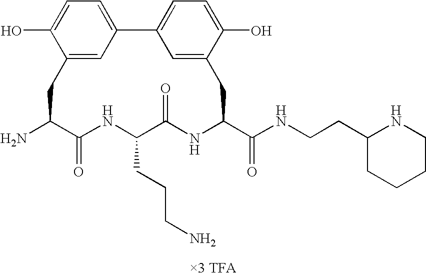 | 128A | LC-MS (Method 20): Rₜ = 2.15 min. MS (ESI): m/z = 567 (M− 3TFA + H)⁺. |
| 37 | 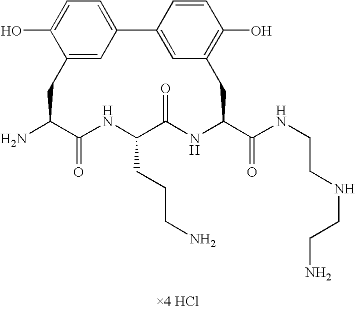 | 135A | LC-MS (Method 20): Rₜ = 1.31 min. MS (EI): m/z = 542 (M − 4HCl + H)⁺ |
| 38 | 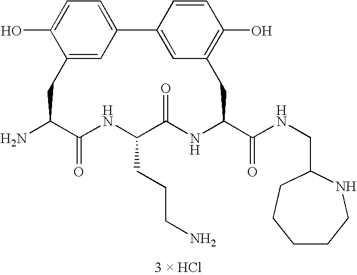 | 130A | LC-MS (Method 20): Rₜ = 2.26 min. MS (EI): m/z = 567 (M − 3HCl + H)⁺ |
| 39 | 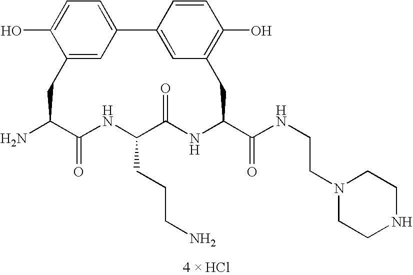 | 131A | LC-MS (Method 13): Rₜ = 3.79 min. MS (EI): m/z = 568 (M − 4HCl + H)⁺ |
| 40 | 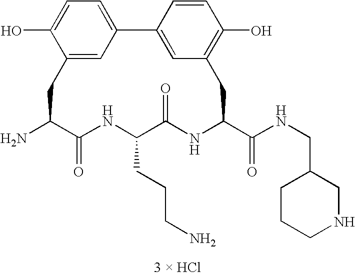 | 132A | LC-MS (Method 20): Rₜ = 2.00 min. MS (EI): m/z = 552 (M − 3HCl + H)⁺ |
| 41 | 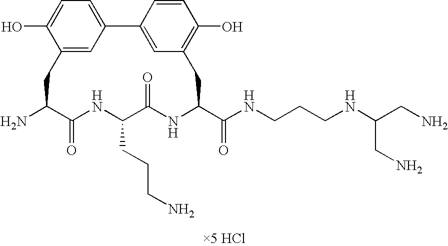 | 134A | LC-MS (Method 20): Rₜ = 1.75 min. MS (EI): m/z = 585 [M − 5HCl + H]⁺1H-NMR (400 MHz, D₂O): δ = 1.5-1.7 (m, 8H), 2.70-3.65 (m, 20H), 4.43 (m, 1H), 6.89 (d, 2H), 6.97 (s, 1H), 7.26 (s, 1H), 7.36 (d, 1H), 7.43 (d, 1H), 7.83 (s, 1H). |

Example 42 detailed in the following table is prepared in analogy to the method of Example 32

| | | Precursor of | |
|---|---|---|---|
| Example | | Example | |
| No. | Structure | No | Analytical data |
| 42 | 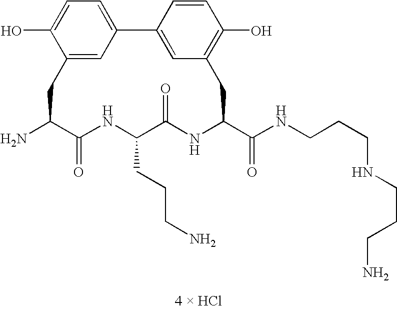 | 127A | HPLC (Method 22): Rₜ = 3.02 min. LC-MS (Method 20), Rₜ = 1.08 min MS (EI): m/z = 569 (M − 4HCl + H)⁺ |

### Example 43

### (8S,11S,14S)-14-Amino-1-(3-aminopropyl)-N-{2-[bis(2-aminoethyl)amino]ethyl}-5,17-dihydroxy-10,13-dioxo-9,12-diazatricyclo[14.3.1.1^{2,6}]henicosa-1(20),2(21),3,5,16,18-hexaene-8-carboxamide tetrahydrochloride

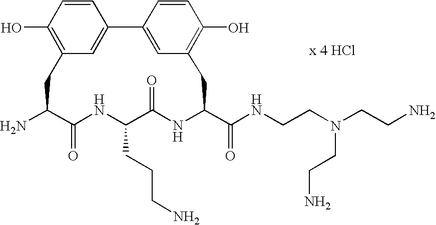

9 mg (0.01 mmol) of the compound from Example 136A are suspended in 8 ml of glacial acetic acid/ethanol/water (4/1/1) and mixed with 5 mg of palladium on activated carbon (10%). Hydrogenation is carried out under atmospheric pressure overnight, the mixture is filtered through kieselguhr, and the mother liquor is concentrated in vacuo. The residue is mixed with 0.1N hydrochloric acid and again concentrated. Drying in vacuo results in the desired title compound.

Yield: 6.6 mg (100% of theory)

LC-MS (Method 20): Rₜ=1.36 min

MS (EI): m/z=585 (M-4HCl+H)⁺

### Example 44

### (8S,11S,14S)-14-Amino-N-[(1S)-4-amino-1-(hydroxymethyl)butyl]-11-(3-aminopropyl)-5,17-dihydroxy-10,13-dioxo-9,12-diazatricyclo[14.3.1.1^{2,6}]henicosa-1(20),2(21),3,5,16,18-hexaene-8-carboxamide tri(trifluoroacetate)

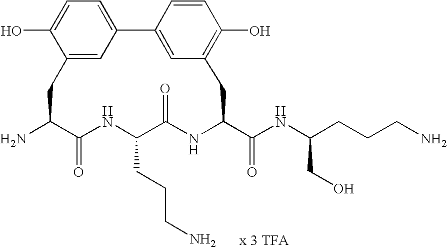

68 mg (0.079 mmol) of tert-butyl {3-[(8S,11S,14S)-14-[(tert-butoxycarbonyl)amino]-8-({[(1R)-4-[(tert-butoxycarbonyl)amino]-1-(hydroxymethyl)butyl]amino}carbonyl)-5,17-dihydroxy-10,13-dioxo-9,12-diazatricyclo[14.3.1.1^{2,6}]henicosa-1(20),2(21),3,5,16,18-hexaen-11-yl]propyl}-carbamate (Example 152A) are put into 0.7 ml of 4N hydrogen chloride solution in dioxane while cooling in ice. The ice bath is removed and the mixture is stirred at room temperature for 2 h. The solvent is evaporated in vacuo, and the remaining solid is converted into the tri(trifluoroacetate) by preparative HPLC (Reprosil ODS-A, mobile phase acetonitrile/0.2% aqueous trifluoroacetic acid 5:95→95:5).

Yield: 3.4 mg (5% of theory)

LC-MS (Method 20): Rₜ=1.95 min

MS (EI): m/z 557 (M-3TFA+H)⁺

### Example 45

### (8S,11S,14S)-14-Amino-11-(3-aminopropyl)-N-[(2S)-2,5-diaminopentyl]-5,17-dihydroxy-10,13-dioxo-9,12-diazatricyclo[14.3.1.1^{2,6}]henicosa-1(20),2(21),3,5,16,18-hexaene-8-carboxamide tetrahydrochloride

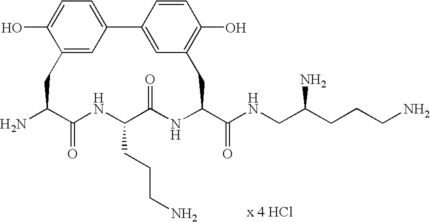

95 mg (0.10 mmol) of tert-butyl {3-[(8S,11S,14S)-8-[({(2S)-2,5-bis[(tert-butoxycarbonyl)amino]pentyl}amino)carbonyl]-14-[(tert-butoxycarbonyl)amino]-5,17-dihydroxy-10,13-dioxo-9,12-diazatricyclo[14.3.1.1^{2,6}]henicosa-1(20),2(21),3,5,16,18-hexaen-11-yl]propyl}carbamate (Example 153A) are put into 12 ml of an ice-cold 4N hydrogen chloride/dioxane solution. The ice bath is removed and the mixture is stirred at room temperature for one hour. It is then evaporated to dryness in vacuo and dried to constant weight.

Yield: 61 mg (88% of theory)

LC-MS (Method 20): Rₜ=0.85 min

MS (EI): m/z=556 (M-4HCl+H)⁺

¹H-NMR (400 MHz; D₂O): δ=1.55-1.95 (m, 8H), 2.82-3.08 (m, 6H), 3.22 (m_{c}, 1H), 3.35-3.75 (m, 4H), 4.33 (m_{c}, 1H), 4.42 (m_{c}, 1H), 4.78 (m_{c}, 1H), 6.83-6.90 (m, 2H), 6.95 (s, 1H), 7.23 (s, 1H), 7.32 (d, 1H), 7.40 (d, 1H).

Examples 46 to 58 listed in the following table are prepared in analogy to the method of Example 6 or 16 from the appropriate starting compounds:

| | | | |
|---|---|---|---|
| Exam- | | Precursor of | |
| ple | | Example | |
| No. | Structure | No | Analytical data |

| | | | |
|---|---|---|---|
| 46 | 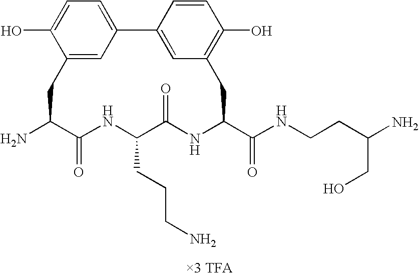 | 176A | LC-MS (Method 20): Rₜ = 2.09 min. MS (ESI): m/z = 543 (M − 3TFA + H)⁺¹H-NMR (400 MHz, D₂O): δ = 1.5-1.9 (m, 6H), 2.85 (m_{c}, 1H), 2.93 (t, 2H), 3.03 (m_{c}, 1H), 3.17 (m_{c}, 1H), 3.2-3.4 (m, 3H), 3.5-3.65 (m, 2H), |
| #3.77 (m_{c}, 1H), 4.42 (m_{c}, 1H), 4.6-4.8 (m, 2H, under D₂O), 6.85-6.91 (m, 2H), 6.96 (s, 1H), | | | |
| #7.25 (s, 1H), 7.34 (d, 1H), 7.43 (d, 1H). | | | |
| 47 | 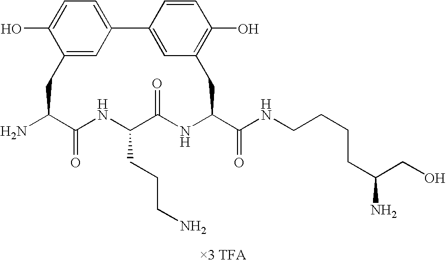 | 177A | LC-MS (Method 20): Rₜ = 2.04 min. MS (ESI): m/z = 571 (M − 3TFA + H)⁺. ¹H-NMR (400 MHz, D₂O): δ =1.45 (m_{c}, 2H), 1.5-1.9 (m, 8H), 2.85 (m_{c}, 1H), 2.94 (t, 2H), 3.03 (m_{c}, 1H), |
| #3.13 (m_{c}, 1H), 3.18-3.33 (m, 3H), 3.5-3.65 (m, 2H), 3.77 (m_{c}, 1H), 4.4 (m_{c}, 1H), 4.6-4.8 (m, 2H, under | | | |
| #D₂O), 6.85-6.91 (m, 2H), 6.96 (s, 1H), 7.25 (s, 1H), 7.34 (d, 1H), 7.42 (d, 1H). | | | |
| 48 | 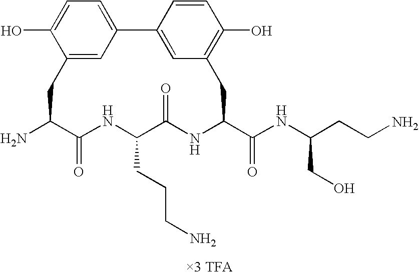 | 178A | LC-MS (Method 20): Rₜ =2.08 min. MS (ESI): m/z = 543 (M −3TFA + H)⁺. |
| 49 | 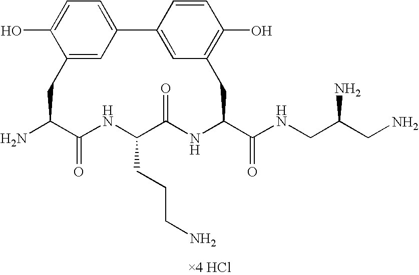 | 181A | MS (ESI): m/z = 528 (M − 4HCl + H)⁺¹H-NMR (400 MHz, D₂O): δ = 1.55-1.9 (m, 4H), 2.8-3.1 (m, 4H), 3.2-3.4 (m, 3H), 3.5-3.75 (m, 4H), 4.42 (m_{c}, 1H), 4.6 (m, 1H, under D₂O), 4.81 (m_{c}, 1H), 6.83-6.92 (m, 2H), 6.97 (s, 1H), 7.25 (s, 1H), 7.36 (d, 1H), 7.43 (d, 1H). |
| 50 | 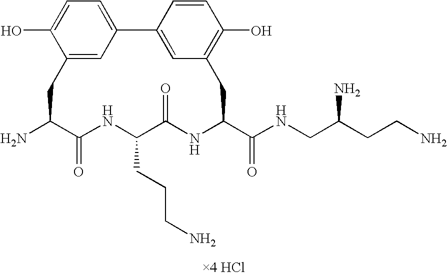 | 180A | MS (ESI: m/z = 542 (M − 4HCl + H)⁺¹H-NMR (400 MHz, D₂O): δ = 1.55-1.9 (m, 4H), 1.92-2.04 (m, 2H), 2.82-3.15 (m, 6H), 3.22 (m_{c}, 1H), 3.45-3.75 (m, 4H), 4.42 (m_{c}, 1H), 4.68 (m, 1H, under D₂O), 4.79 (m_{c}, 1H), 6.83-6.92 (m, 2H), 6.97 (s, 1H), 7.25 (s, 1H), 7.36 (d, 1H), |
| #7.43 (d, 1H). | | | |
| 51 | 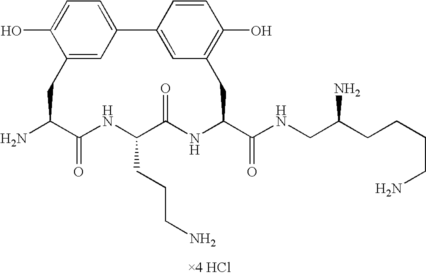 | 182A | MS (ESI): m/z = 570 (M − 4HCl + H)⁺¹H-NMR (400 MHz, D₂O): δ 1.45-1.9 (m, 10H), 2.82-3.15 (m, 6H), 3.22 (m_{c}, 1H), 3.45-3.75 (m, 4H), 4.42 (m_{c}, 1H), 4.68 (m, 1H, under D₂O, 4.77 (m_{c}, 1H), 6.83-6.92 (m, 2H), 6.97 (s, 1H), 7.25 (s, 1H), 7.36 (d, 1H), 7.43 (d, 1H). |
| 52 | 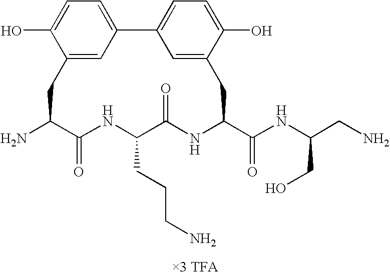 | 179A | LC-MS (Method 20): Rₜ = 2.01 min. MS (ESI): m/z = 529 (M − 3TFA + H)⁺. ¹H-NMR (400 MHz, D₂O): δ = 1.5-1.9 (m, 4H), 2.8-3.3 (m, 7H), 3.4-3.75 (m, 3H), 4.18 (m_{c}, 1H), 4.41 (m_{c}, 1H) 4.6 (m, 1H, under D₂O), 4.78 (m_{c}, 1H), 6.83-6.9 (m, 2H), 6.96 (s, 1H), 7.25 (s, |
| #1H), 7.34 (d, 1H), 7.42 (d, 1H). | | | |
| 53 | 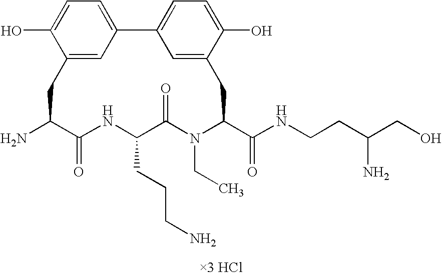 | 183A | LC-MS (Method 20): Rₜ = 2.33 min. MS (ESI): m/z = 571 (M − 3HCl + H)⁺. ¹H-NMR (400 D₂O): δ 0.96 (t, 3H), 1.75-2.1 (m, 6H), 3.06 (t, 2H), 3.1-3.9 (m, 11H), 4.52 (m_{c}, 1H), 5.05 (m_{c}, 1H), 5.76 (m_{c}, 1H), 6.95-7.03 (m, 2H), 7.14 (s, 1H), 7.28 (s, 1H), 7.54 (d, 1H), |
| #7.6 (d, 1H). | | | |
| 54 | 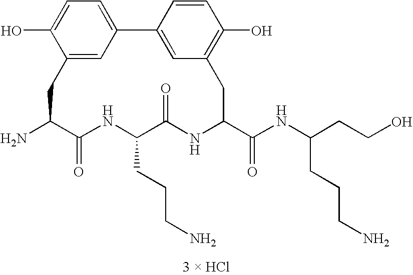 | 184A | LC-MS (Method 20): Rₜ = 1.96 min. MS (EI): m/z = 571 [M-3HCl + H]⁺¹H-NMR (400 MHz, D₂O): δ = 1.7-1.90 (m, 10H), 2.94-3.14 (m, 6 H), 3.32 (m, 1H, 3.60-3.84 (m, 5H), 3.99 (m, 1H), 4.51 (m, 1H), 6.99 (d, 2H), 7.07 (s, 1H), 7.37 (s, 1H), 7.46 (d, 1H), 7.53 (d, 1H). |
| 55 | 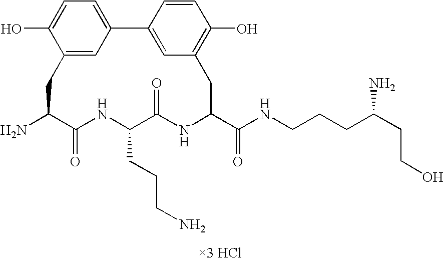 | 186A | LC-MS (Method 20): Rₜ = 2.23 min. MS (EI): m/z = 571 [M − 3HCl + H]⁺1H-NMR (400 MHz, D₂O): δ= 1.4-1.9 (m, 12H), 2.70-3.7 (m, 14H), 4.44 (m, 1H), 6.90 (d, 2H), 6.98 (s, 1H), 7.28 (s, 1H), 7.36 (d, 1H), 7.44 (d, lH), 7.44 (s, 1H). |
| 56 | 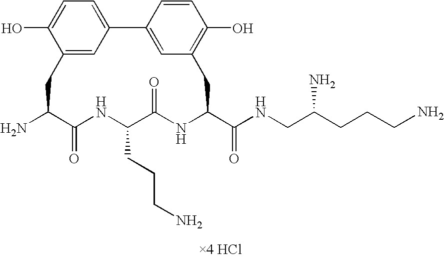 | 192A | MS (ESI): m/z = 556 (M − 4HCl + H)¹H-NMR (400 MHz, D₂O): δ = 1.55-1.95 (m, 8H), 2.82-3.08 (m, 6H), 3.22 (m_{c}, 1H), 3.35-3.85 (m, 4H), 4.41 (m_{c}, 1H), 4.7 (m, 1H under D₂O), 4.78 (m_{c}, 1H), 6.83-6.90 (m, 2H), 6.95 (s, 1H), 7.23 (s, 1H), 7.32 (d, 1H), 7.40 (d, 1H). |
| 57 | 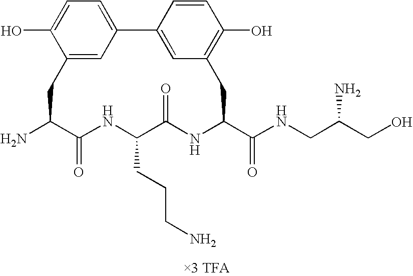 | 174A | LC-MS (Method 20): Rₜ = 2.14 min MS (ESI): m/z = 529 (M − 3TFA + H)⁺¹H-NMR (400 MHz, D₂O): δ = 1.5-1.8 (m, 4H), 2.75-3.0 (m, 4H), 3.17 (m_{c}, 1H), 3.35-3.85 (m, 6H), 4.43 (m_{c}, 1H), 4.55 (m_{c}, 1H), 4.7 (m, 1H under D₂O), 6.80 (m_{c}, 2H), 6.89 (s, 1H), 7.18 (s, |
| #1H), 7.28 (d, 1H), 7.35 (d, 1H). | | | |
| 58 | 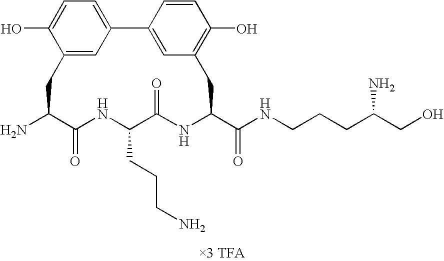 | 175A | LC-MS (Method 20): Rₜ = 1.95 min MS (ESI: m/z = 557 (M − 3TFA + H)⁺¹H-NMR (400 MHz, D₂O): δ 1.5-1.85 (m, 8H), 2.78-3.08 (m, 4H), 3.1-3.33 (m, 4H), 3.48-3.85 (m, 3H), 4.41 (m_{c}, 1H), 4.6-4.7 (m, 2H under D₂O, 6.83-6.90 (m, 2H), 6.94 (s, 1H), 7.23 (s, 1H), 7.32 (d, 1H), |
| #7.40 (d, 1H). | | | |

B. Assessment of the Physiological Activity

Abbreviations used:

| | |
|---|---|
| AMP | adenosine monophosphate |
| ATP | adenosine triphosphate |
| BHI medium | brain heart infusion medium |
| CoA | coenzyme A |
| DMSO | dimethyl sulphoxide |
| DTT | dithiothreitol |
| EDTA | ethylenediaminetetraacetic acid |
| KCI | potassium chloride |
| KH₂PO₄ | potassium dihydrogen phosphate |
| MgSO₄ | magnesium sulphate |
| MIC | minimum inhibitory concentration |
| MTP | microtitre plate |
| NaCl | sodium chloride |
| Na₂HPO₄ | disodium hydrogenphosphate |
| NH₄Cl | ammonium chloride |
| NTP | nucleotide triphosphate |
| PBS | phosphate-buffered saline |
| PCR | polymerase chain reaction |
| PEG | polyethylene glycol |
| PEP | phosphoenolpyruvate |
| Tris | tris[hydroxymethyl)aminomethane |

The in vitro effect of the compounds of the invention can be shown in the following assays:

In vitro Transcription-Translation with E. coli Extracts

An S30 extract is prepared by harvesting logarithmically growing Escherichia coli MRE 600 (M. Müller; Freiburg University), washing and employing as described for the in vitro transcription-translation assay (Müller, M. and Blobel, G. Proc Natl Acad Sci USA (1984) 81, pp. 7421-7425).

1 μl of cAMP (11.25 mg/ml) are additionally added per 50 μl of reaction mix to the reaction mix for the in vitro transcription-translation assay. The assay mixture amounts to 105 μl, with 5 μl of the substance to be tested being introduced in 5% strength DMSO. 1 μg/100 μl of mixture of the plasmid pBESTluc (Promega, Germany) are used as transcription templates. After incubation at 30° C. for 60 min, 50 μl of luciferin solution (20 mM tricine, 2.67 mM MgSO₄, 0.1 mM EDTA, 33.3 mM DTT pH 7.8, 270 μM CoA, 470 μM luciferin, 530 μM ATP) are added, and the resulting bioluminescence is measured in a luminometer for 1 minute. The IC₅₀ is indicated by the concentration of an inhibitor which leads to 50% inhibition of the translation of firefly luciferase.

In vitro Transcription-Translation with S. aureus Extracts

Construction of an S. aureus Luciferase Reporter Plasmid

A reporter plasmid which can be used in an in vitro transcription-translation assay from S. aureus is constructed by using the plasmid pBESTluc (Promega Corporation, USA). The E. coli tac promoter present in this plasmid in front of the firefly luciferase is replaced by the capA1 promoter with appropriate Shine-Dalgarno sequence from S. aureus. The primers CAPFor 5′-CGGCCAAGCTTACTCGGAT-CCAGAGTTTGCAAAATATACAGGGGATTATATATAATGGAAAACAAGAA AGGAAAATAGGAGGTTTATATGGAAGACGCCA-3′ and CAPRev 5′-GTCATCGTCGGGAAGACCTG-3′ are used for this. The primer CAPFor contains the capA1 promoter, the ribosome binding site and the 5′ region of the luciferase gene. After PCR using pBESTluc as template it is possible to isolate a PCR product which contains the firefly luciferase gene with the fused capA1 promoter. This is, after restriction with ClaI and HindIII, ligated into the vector pBESTluc which has likewise been digested with ClaI and HindIII. The resulting plasmid pla is able to replicate in E. coli and be used as template in the S. aureus in vitro transcription-traslation assay.

Preparation of S30 Extracts from S. aureus

Six litres of BHI medium are inoculated with a 250 ml overnight culture of an S. aureus strain and allowed to grow at 37° C. until the OD600 run is 2-4. The cells are harvested by centrifugation and washed in 500 ml of cold buffer A (10 mM Tris acetate, pH 8.0, 14 mM Magnesium acetate, 1 mM DTT, 1 M KCl). After renewed centrifugation, the cells are washed in 250 ml of cold buffer A with 50 mM KCl, and the resulting pellets are frozen at −20° C. for 60 min. The pellets are thawed on ice in 30 to 60 min and taken up to a total volume of 99 ml in buffer B (10 mM Tris acetate, pH 8.0, 20 mM Magnesium acetate, 1 mM DTT, 50.mM KCl). 1.5 ml portions of lysostaphin (0.8 mg/ml) in buffer B are introduced into 3 precooled centrifuge cups and each mixed with 33 ml of the cell suspension. The samples are incubated at 37° C., shaking occasionally, for 45 to 60 min, before 150 μl of a 0.5 M DTT solution are added. The lysed cells are centrifuged at 30 000×g and 4° C. for 30 min. The cell pellet is taken up in buffer B and then centrifuged again under the same conditions, and the collected supernatants are combined. The supernatants are centrifuged again under the same conditions, and 0.25 volume of buffer C (670 mM Tris acetate, pH 8.0, 20 mM Magnesium acetate, 7 mM Na₃ phosphoenolpyruvate, 7 mM DTT, 5.5 mM ATP, 70 μM amino acids (complete from Promega), 75 μg of pyruvate kinase (Sigma, Germany)/ml are added to the upper ⅔ of the supernatant. The samples are incubated at 37° C. for 30 min. The supernatants are dialysed against 2 l of dialysis buffer (10 mM Tris acetate, pH 8.0, 14 mM Magnesium acetate, 1 mM DTT, 60 mM Potassium acetate) in a dialysis tube with a 3500 Da cut-off with one buffer change at 4° C. overnight. The dialysate is concentrated to a protein concentration of about 10 mg/ml by covering the dialysis tube with cold PEG 8000 powder (Sigma, Germany) at 4° C. The S30 extracts can be stored in aliquots at −70° C.

Determination of the IC₅₀ in the S. aureus in vitro Transcription-Translation Assay Inhibition of protein biosynthesis of the compounds can be shown in an in vitro transcription-translation assay. The assay is based on the cell-free transcription and translation of firefly luciferase using the reporter plasmid pla as template and cell-free S30 extracts obtained from S. aureus. The activity of the resulting luciferase can be detected by luminescence measurement.

The amount of S30 extract or plasmid pla to be employed must be tested anew for each preparation in order to ensure an optimal concentration in the assay. 3 μl of the substance to be tested, dissolved in 5% DMSO, are introduced into an MTP. Then 10 μl of a suitably concentrated plasmid solution pla are added. Then 46 μl of a mixture of 23 μl of premix (500 mM Potassium acetate, 87.5 mM Tris acetate, pH 8.0, 67.5 mM ammonium acetate, 5 mM DTT, 50 μg of folic acid/ml, 87.5 mg of PEG 8000/ml, 5 mM ATP, 1.25 mM each NTP, 20 μM each amino acid, 50 mM PEP (Na₃ salt), 2.5 mM cAMP, 250 μg each E. coli tRNA/ml) and 23 μl of a suitable amount of S. aureus S30 extract are added and mixed. After incubation at 30° C. for 60 min, 50 μl of luciferin solution (20 mM tricine, 2.67 mM MgSO₄, 0.1 mM EDTA, 33.3 mM DTT pH 7.8, 270 μM CoA, 470 μM luciferin, 530 μM ATP) are added, and the resulting bioluminescence is measured in a luminometer for 1 min. The IC₅₀ is indicated as the concentration of an inhibitor which leads to 50% inhibition of the translation of firefly luciferase.

Determination of the Minimum Inhibitory Concentration (MIC)

The minimum inhibitory concentration (MIC) is the minimum concentration of an antibiotic with which the growth of a test microbe is inhibited over 18-24 h. The inhibitor concentration can in these cases be determined by standard microbiological methods (see, for example, The National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The MIC of the compounds of the invention is determined in the liquid dilution test on the 96-well microtitre plate scale. The bacterial microbes are cultivated in a minimal medium (18.5 mM Na₂HPO₄, 5.7 mM KH₂PO₄, 9.3 mM NH₄Cl, 2.8 mM MgSO₄, 17.1 mM NaCl, 0.033 μg/ml thiamine hydrochloride, 1.2 μg/ml nicotinic acid, 0.003 μg/ml biotin, 1% glucose, 25 μg/ml of each proteinogenic amino acid with the exception of phenylalanine; [H.-P. Kroll; unpublished]) with addition of 0.4% BH broth (test medium). In the case of Enterococcus faecium L4001, heat-inactivated fetal calf serum (FCS; GibcoBRL, Germany) is added to the test medium in a final concentration of 10%. Overnight cultures of the test microbes are diluted to an OD₅₇₈ of 0.001 (to 0.01 in the case of enterococci) in fresh test medium, and incubated 1:1 with dilutions of the test substances (1:2 dilution steps) in test medium (200 μl final volume). The cultures are incubated at 37° C. for 18-24 hours; enterococci in the presence of 5% CO₂.

The lowest substance concentration in each case at which bacterial growth was no longer visible is defined as the MIC. The MIC values in μM of some compounds of the invention for a series of test microbes are listed by way of example in the table below. The compounds show a graded antibacterial effect against most of the test microbes.

TABLE A
(with Comparative Example 20A (biphenomycin B))

| | | | MIC | IC₅₀ |
|---|---|---|---|---|
| Ex. | MIC | MIC | E. faecium | S. aureus 133 |
| No. | S. aureus E33 | S. aureus T17 | L4001 | Translation |
| 1 | 3.1 | 3.1 | 12.5 | 0.1 |
| 2 | 1.6 | 6.3 | 25 | 0.2 |
| 3 | 1.6 | 3.1 | 25 | 0.35 |
| 6 | 3.1 | 3.1 | >50 | 0.5 |
| 20A | 0.1 | >25 | >25 | 1.5 |

All concentration data in μM.

Alternative Method for Determining the Minimum Inhibitory Concentration (MIC)

The minimum inhibitory concentration (MIC) is the minimum concentration of an antibiotic with which the growth of a test microbe is inhibited over 18-24 h. The inhibitor concentration can be determined by standard microbiological methods using modified medium in an agar dilution test (see, for example, the National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The bacterial microbes are cultivated on 1.5% agar plates containing 20% defibrinated horse blood. The test microbes, which are incubated on Columbia blood agar plates (Becton-Dickinson) overnight, are diluted in PBS, adjusted to a microbe count of about 5×10⁵ microbes/ml and placed as drops (1-3 μl) on test plates. The test substances contain various dilutions of the test substances (1:2 dilution stages). The cultures are incubated at 37° C. in the presence of 5% CO₂ for 18-24 hours.

The lowest concentration of each substance at which no visible bacterial growth occurs is defined as the MIC and is reported in μg/ml.

TABLE B
(with Comparative Example 20A (biphenomycin B))

| | | | MIC | IC₅₀ |
|---|---|---|---|---|
| Ex. | MIC | MIC | E. faecium | S. aureus |
| No. | S. aureus E33 | S. aureus T17 | L4001 | 133 Translation |
| 1 | 4 | 4 | 16 | 0.1 |
| 3 | 2 | 4 | 16 | 0.35 |
| 6 | 0.5 | 2 | 8 | 0.5 |
| 42 | 1 | 2 | 4 | 0.4 |
| 43 | 1 | 2 | 16 | 0.15 |
| 45 | 0.5 | 2 | 2 | 0.1 |
| 20A | <0.03 | >32 | 0.5 | 1.5 |

Concentration data: MIC in μg/ml; IC₅₀ in μM.

Systemic Infection with S. aureus 133

The suitability of the compounds of the invention for treating bacterial infections can be shown in various animal models. For this purpose, the animals are generally infected with a suitable virulent microbe and then treated with the compound to be tested, which is in a formulation which is adapted to the particular therapy model. The suitability of the compounds of the invention can be demonstrated specifically for the treatment of bacterial infections in a mouse sepsis model after infection with S. aureus.

For this purpose, S. aureus 133 cells are cultured overnight in BH broth (Oxoid, Germany). The overnight culture was diluted 1:100 in fresh BH broth and expanded for 3 hours. The bacteria which are in the logarithmic phase of growth are centrifuged and washed twice with buffered physiological saline solution. A cell suspension in saline solution with an extinction of 50 units is then adjusted in a photometer (Dr Lange LP 2W). After a dilution step (1:15), this suspension is mixed 1:1 with a 10% strength mucine suspension. 0.2 ml of this infection solution is administered i.p. per 20 g of mouse. This corresponds to a cell count of about 1-2×10 microbes/mouse. The i.v. therapy takes place 30 minutes after the infection. Female CFW1 mice are used for the infection test. The survival of the animals is recorded for 6 days. The animal model is adjusted so that untreated animals die within 24 h after the infection. It was possible to demonstrate in this model a therapeutic effect of ED₁₀₀=1.25 mg/kg for the compound of Example 2.

Determination of the Rates of Spontaneous Resistance to S. aureus

The spontaneous resistance rates for the compounds of the invention are determined as follows: the bacterial microbes are cultivated in 30 ml of a minimal medium (18.5 mM Na₂HPO₄, 5.7 mM KH₂PO₄, 9.3 mM NH₄Cl, 2.8 mM MgSO₄, 17.1 mM NaCl, 0.033 μg/ml thiamine hydrochloride, 1.2 μg/ml nicotinic acid, 0.003 μg/ml biotin, 1% glucose, 25 μg/ml of each proteinogenic amino acid with the addition of 0.4% BH broth) at 37° C. overnight, centrifuged at 6000×g for 10 min and resuspended in 2 ml of phosphate-buffered physiological NaCl solution (about 2×10⁹ microbes/ml). 100 μl of this cell suspension, and 1:10 and 1:100 dilutions, are plated out on predried agar plates (1.5% agar, 20% defibrinated horse blood, or 1.5% agar, 20% bovine serum in 1/10 Müller-Hinton medium diluted with PBS) which contain the compound of the invention to be tested in a concentration equivalent to 5×MIC or 10×MIC, and incubated at 37° C. for 48 h. The resulting colonies (cfu) are counted.

TABLE C
Rates of spontaneous resistance for the compounds of the
invention (with Comparative Example 20A (biphenomycin B))

| Ex. No. | 6 | 6 | 1 | 20A |
|---|---|---|---|---|
| (concentration) | (5 × MIC) | (10 × MIC) | (10 × MIC) | (10 × MIC) |
| S. aureus 133 | <8 × 10⁻⁹* | <8 × 10⁻⁹ | <4 × 10⁻⁸ | <1.7 × 10⁻⁶ |
| S. aureus T17 | n.d. | n.d. | <4 × 10⁻⁸ | n.d. |
| S. aureus | <6 × 10⁻⁹* | <6 × 10⁻⁹ | <6 × 10⁻⁸ | n.d. |
| RN4220 | | | | |
| S. aureus | <8 × 10⁻⁹* | <4 × 10⁻⁹ | <6 × 10⁻⁸ | n.d. |
| RN4220Bi^{R} | | | | |
| S. pneumoniae | <4 × 10⁻⁸* | n.d. | n.d. | n.d. |
| G9a | | | | |
| E. faecalis | <4 × 10⁻⁹* | <4 × 10⁻¹⁰* | n.d. | 4.1 × 10⁻⁶ |
| ICB 27159 | | | | |
| E. faecium | 4 × 10⁻⁸ | <4 × 10⁻⁹ | n.d. | n.d. |
| L4001 | | | | |

*Colonies with increased MIC (4-8-fold) were isolated.
n.d. not determined.

Isolation of the biphenomycin-Resistant S. aureus Strains RN4220Bi^{R} and T17

The S. aureus strain RN4220Bi^{R} is isolated in vitro. For this purpose, 100 μl portions of an S. aureus RN4220 cell suspension (about 1.2×10⁸ cfu/ml) are plated out on an antibiotic-free agar plate (18.5 mM Na₂HPO₄, 5.7 mM KH₂PO₄, 9.3 mM NH₄Cl, 2.8 mM MgSO₄, 17.1 mM NaCl, 0.033 μg/ml thiamine hydrochloride, 1.2 μg/ml nicotinic acid, 0.003 μg/ml biotin, 1% glucose, 25 μg/ml of each proteinogenic amino acid with the addition of 0.4% BH broth and 1% agarose) and on an agar plate containing 2 μg/ml biphenamycin B (10×MIC), and incubated at 37° C. overnight. Whereas about 1×10⁷ cells grow on the antibiotic-free plate, about 100 colonies grow on the antibiotic-containing plate, corresponding to a resistance rate of 1×10⁻⁵. Some of the colonies grown on the antibiotic-containing plate are tested for the biphenomycin B MIC. One colony with an MIC of >50 μM is selected for further use, and the strain is referred to as RN4220Bi^{R}.

The S. aureus strain T17 is isolated in vivo. CFW1 mice are infected intraperitoneally with 4×10⁷ S. aureus 133 cells per mouse. 0.5 h after the infection, the animals are treated intravenously with 50 mg/kg biphenomycin B. The kidneys are removed from the surviving animals on day 3 after the infection. After homogenization of the organs, the homogenates are plated out as described for RN4220Bi^{R} on antibiotic-free and antibiotic-containing agar plates and incubated at 37° C. overnight. About half the colonies isolated from the kidney show growth on the antibody-containing plates (2.2×10⁶ colonies), demonstrating the accumulation of biphenomycin B-resistant S. aureus cells in the kidney of the treated animals. About 20 of these colonies are tested for the biphenomycin B MIC, and a colony with an MIC of >50 μM is selected for further cultivation, and the strain is referred to a T17.

C. Exemplary Embodiments of Pharmaceutical Compositions

The compounds of the invention can be converted into pharmaceutical preparations in the following way:

Solution which can be Administered Intravenously;

Composition:

1 mg of the compound of Example 1, 15 g of polyethylene glycol 400 and 250 g of water for injections.

Preparation:

The compound of the invention is dissolved together with polyethylene glycol 400 in the water with stirring. The solution is sterilized by filtration (pore diameter 0.22 μm) and dispensed under aseptic conditions into heat-sterilized infusion bottles. These are closed with infusion stoppers and crimped caps.

1. Compound of the formula 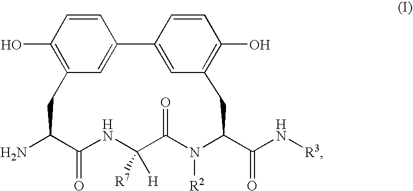
in which
R⁷ is a group of the formula 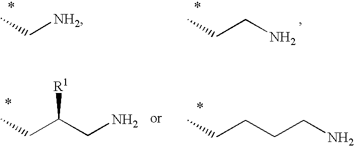
where
R¹ is hydrogen or hydroxy,
is the point of attachment to the carbon atom,
R² is hydrogen, methyl or ethyl,
R³ is a group of the formula 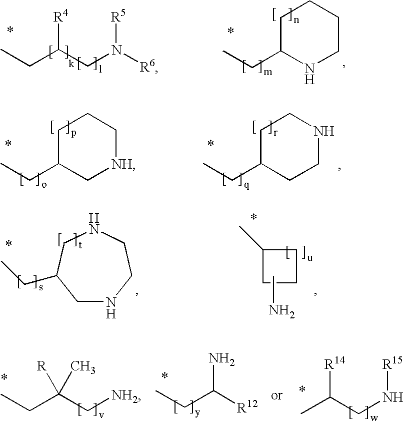
where
is the point of attachment to the nitrogen atom,
R⁴ is hydrogen or hydroxy,
R⁵ and R¹⁵ are independently of one another hydrogen, methyl or a group of the formula 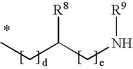
in which
is the point of attachment to the nitrogen atom,
R⁸ is hydrogen or *—(CH₂)_{f}—NHR¹⁰,
in which
R¹⁰ is hydrogen or methyl,
and
f is a number 1, 2 or 3,
R⁹ is hydrogen or methyl,
d is a number 0, 1, 2 or 3,
and
e is a number 1, 2 or 3,
R⁶ is hydrogen or aminoethyl,
or
R⁵ and R⁶ form together with the nitrogen atom to which they are bonded a piperazine ring,
R¹² and R¹⁴ are independently of one another a group of the formula *—(CH₂)_{Z1}—OH or *—(CH₂)_{Z2}—NHR¹³,
in which
is the point of attachment to the carbon atom,
Z1 and Z2 are independently of one another a number 1, 2, 3 or 4,
R¹³ is hydrogen or methyl,
k and t are independently of one another a number 0 or 1,
l, w and y are independently of one another a number 1, 2, 3 or 4,
m, r, s and v are independently of one another a number 1 or 2,
n, o, p and q are independently of one another a number 0, 1 or 2,
u is a number 0, 1, 2 or 3, 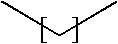
w or y may independently of one another when w or y is 3 carry a hydroxy group on the middle carbon atom of the three-membered chain,
or one of the salts thereof, the solvates thereof or the solvates of the salts thereof.

2. Compound according to claim 1, characterized in that
R⁷ is a group of the formula 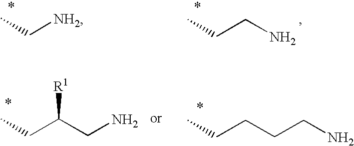
where
R¹ is hydrogen or hydroxy,
is the point of attachment to the carbon atom,
R² is hydrogen, methyl or ethyl,
R³ is a group of the formula 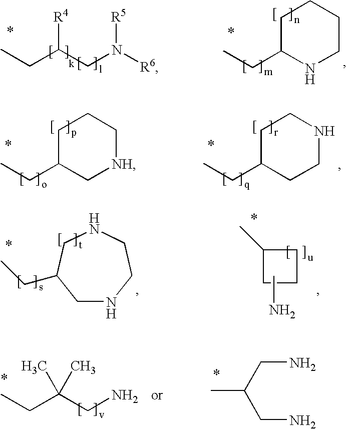
where
R⁴ is hydrogen or hydroxy,
R⁵ is hydrogen or methyl,
R⁶ is hydrogen,
or
R⁵ and R⁶ form together with the nitrogen atom to which they are bonded a piperazine ring
k and t are independently of one another a number 0 or 1,
l is a number 1, 2, 3 or4,
m, r, s and v are independently of one another a number 1 or 2,
n, o, p and q are independently of one another a number 0, 1 or 2,
u is a number 0, 1, 2 or 3,
is the point of attachment to the nitrogen atom,
or one of the salts thereof, the solvates thereof or the solvates of the salts thereof.

3. Compound according to claim 1, characterized in that it corresponds to the formula 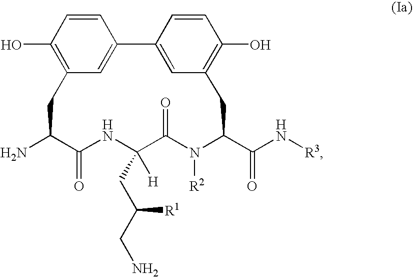
in which
R¹ is hydrogen or hydroxy,
R² is hydrogen or methyl,
R³ is a group of the formula 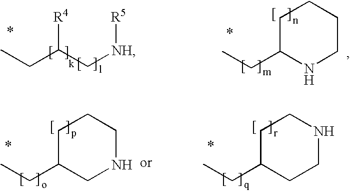
where
R⁴ is hydrogen or hydroxy,
R⁵ is hydrogen or methyl,
k is a number 0 or 1,
l, m and r are independently of one another a number 1 or 2,
n, o, p and q are independently of one another a number 0, 1 or 2,
is the point of attachment to the nitrogen atom,
or one of the salts thereof, solvates thereof or the solvates of the salts thereof.

4. Compound according to any of claim 1, characterized in that
R¹ is hydrogen or hydroxy,
R² is hydrogen or methyl,
R³ is a group of the formula 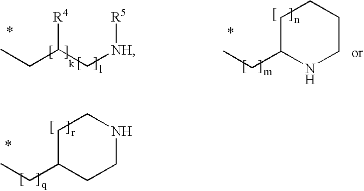
where
R⁴ is hydrogen or hydroxy,
R⁵ is hydrogen or methyl,
k is a number 0 or 1,
l, m and r are independently of one another a number 1 or 2,
n and q are independently of one another a number 0, 1 or 2,
is the point of attachment to the nitrogen atom.

5. Compound according to any of claim 1, characterized in that
R¹ is hydrogen or hydroxy,
R² is hydrogen or methyl,
R³ is a group of the formula 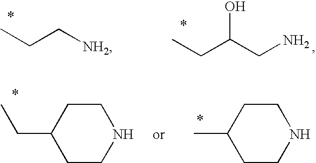
where
is the point of attachment to the nitrogen atom.

6. (8S,11S,14S)-14-Amino-N-(2-aminoethyl)-11-[(2R)-3-amino-2-hydroxy-propyl]-5,17-dihydroxy-9-methyl-10,13-dioxo-9,12-diazatricyclo[14.3.1.1^{2,6}]-henicosa-1(20),2(21),3,5,16,18-hexaene-8-carboxamide of the formula 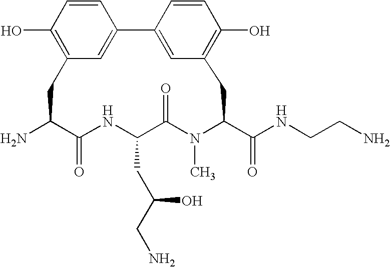
or its trihydrochloride, another of its salts, one of its solvates or one of the solvates of its salts.

7. (8S,11S,14S)-14-Amino-N-(2-aminoethyl)-11-(3-aminopropyl)-5,17-dihydroxy-10,13-dioxo-9,12-diazatricyclo[14.3.1.1^{2,6}]henicosa-1(20),2(21),3,5,16,18-hexaene-8-carboxamide of the formula 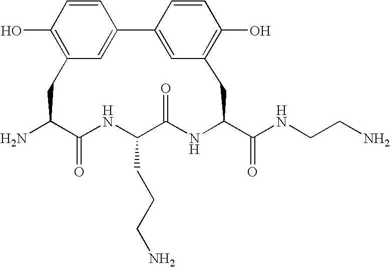
or its trihydrochloride, another of its salts, one of its solvates or one of the solvates of its salts.

8. (8S,11S,14S)-14-Amino-11-(3-aminopropyl)-N-{3-[(3-aminopropyl)amino]propyl}-5,17-dihydroxy-10,13-dioxo-9,12-diazatricyclo[14.3.1.1^{2,6}]henicosa-1(20),2(21),3,5,16,18-hexaene-8-carboxamide of the formula 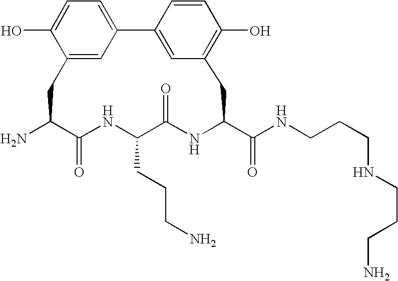
or its tetrahydrochloride, another of its salts, one of its solvates or one of the solvates of its salts.

9. (8S,11S,14S)-14-Amino-11-(3-aminopropyl)-N-{2-[bis(2-aminoethyl)amino]ethyl}-5,17-dihydroxy-10,13-dioxo-9,12-diazatricyclo[14.3.1.1^{2,6}]henicosa-1(20),2(21),3,5,16,18-hexaene-8-carboxamide of the formula 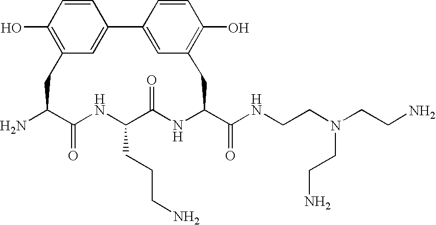
or its tetrahydrochloride, another of its salts, one of its solvates or one of the solvates of its salts.

10. (8S,11S,14S)-14-Amino-11-(3-aminopropyl)-N-[(2S)-2,5-diaminopentyl]-5,17-dihydroxy-10,13-dioxo-9,12-diazatricyclo[14.3.1.1^{2,6}]-henicosa-1(20),2(21),3,5,16,18-hexaene-8-carboxamide of the formula 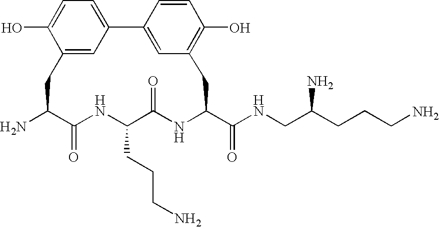
or its tetrahydrochloride, another of its salts, one of its solvates or one of the solvates of its salts.

11. Process for preparing a compound of the formula (I) according to claim 1 or one of its salts, solvates, or the solvates of its salts, characterized in that
[A] a compound of the formula 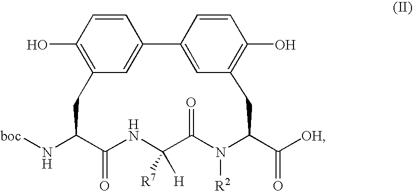
in which R² and R⁷ have the meaning indicated in claim 1, and boc is tert-butoxycarbonyl, is reacted in a two-stage process firstly in the presence of one or more dehydrating reagents with a compound of the formula
H₂NR³ (III),
in which R³ has the meaning indicated in claim 1,
and then with an acid,
or
[B] a compound of the formula 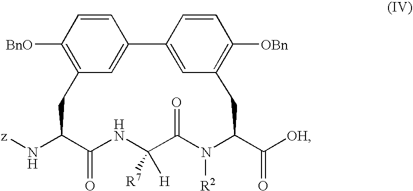
in which R² and R⁷ have the meaning indicated in claim 1, and Z is benzyloxycarbonyl,
is reacted in a two-stage process firstly in the presence of one or more dehydrating reagents with a compound of the formula
H₂NR³ (III),
in which R³ has the meaning indicated in claim 1,
and then with an acid or by hydrogenolysis.

12. Process for preparing a compound of the formula (I) according to claim 1 or one of its solvates, characterized in that a salt of the compound or a solvate of a salt of the compound is converted into the compound by chromatography with addition of a base.

13. (canceled)

14. (canceled)

15. (canceled)

16. Medicament comprising at least one compound according to claim 1 in combination with at least one inert, nontoxic, pharmaceutically suitable excipient.

17. Medicament according to claim 16 for the treatment and/or prophylaxis of bacterial infections.

18. Method for controlling bacterial infections in humans and animals by administration of an antibacterially effective amount of at least one compound according to claim 1 to 10.

19. Method for controlling bacterial infections in humans and animals by administration of an antibacterially effective amount of a medicament according to claim 16.
